(19) United States
(12) Patent Application Publication
(10) Pub. No.: US 2012/0097554 A1
(43) Pub. Date: Apr. 26, 2012

(54) ANALYTE SENSORS COMPRISING ELECTRODES HAVING SELECTED ELECTROCHEMICAL AND MECHANICAL PROPERTIES

(75) Inventors: Rajiv Shah, Rancho Palos Verdes, CA (US); Eric Allan Larson, Simi Valley, CA (US); Katherine T. Wolfe, Dunwoody, GA (US); Daniel E. Pesantez, Canoga Park, CA (US)

(73) Assignee: MEDTRONIC MINIMED, INC., Northridge, CA (US)

(21) Appl. No.: 13/276,183

(22) Filed: Oct. 18, 2011

Related U.S. Application Data
(60) Provisional application No. 61/394,116, filed on Oct. 18, 2010.

(51) Int. Cl. G01N 33/66; A61B 5/1473; G01N 27/327

(52) U.S. Cl. 205/782; 204/403.01; 204/403.14; 600/347

(57) ABSTRACT

Embodiments of the invention disclosed herein comprise amperometric glucose sensor systems that include multiple working electrodes having different material properties as well as algorithms and other elements designed for use with such systems. While embodiments of the innovation can be used in a number of contexts, typical embodiments of the invention include glucose sensors used to facilitate the management of diabetes.

### CROSS REFERENCE TO RELATED APPLICATIONS

This application claims the benefit of U.S. provisional patent application No. 61/394,116, filed Oct. 18, 2010, the entire contents of which are incorporated herein by reference.

### BACKGROUND OF THE INVENTION

1. Field of the Invention

Analyte sensor systems (e.g. glucose sensor systems used in the management of diabetes) and methods and materials for making and using such sensor systems.

2. Description of Related Art

Analyte sensors such as biosensors include devices that use biological elements to convert a chemical analyte in a matrix into a detectable signal. There are many types of biosensors used for a wide variety of analytes. The most studied type of biosensor is the amperometric glucose sensor, which is crucial to the successful glucose level control for diabetes.

A typical glucose sensor works according to the following chemical reactions:

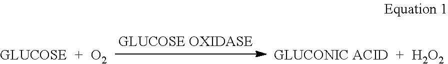

H₂O₂→O₂+2H⁺+2e⁻ Equation 2

The glucose oxidase (GOx) is used to catalyze the reaction between glucose and oxygen to yield gluconic acid and hydrogen peroxide (equation 1). The H₂O₂ reacts electrochemically as shown in equation 2, and the current can be measured by a potentiostat. These reactions, which occur in a variety of oxidoreductases known in the art, are used in a number of sensor designs.

While amperometric sensors are commonly used to monitor glucose, embodiments of these sensors may encounter technical challenges when measuring the broad spectrum of hypoglycemic and hyperglycemic glucose concentrations that can occur in diabetic patients. In addition, amperometric glucose sensors may produce spurious signals in the presence of interferants, compounds that interfere with the measurement of an analyte by generating sensor signals that do not accurately represent the concentration of the analyte being measured. In view of these and other issues, materials, methods and systems designed to enhance amperometric glucose sensor readings are desirable.

### SUMMARY OF THE INVENTION

Embodiments of the invention disclosed herein comprise amperometric glucose sensor systems that include multiple working electrodes having different material properties as well as algorithms designed for use with such systems. As discussed in detail below, systems having the constellations of elements disclosed herein provide a number of advantages over conventional sensor systems and, for example, can be used to enhance sensor accuracy and reliability as well as to address a number of technical challenges observed in this field.

The invention disclosed herein has a number of embodiments. One illustrative embodiment is an amperometric glucose sensor system comprising a processor that is operably coupled to a plurality of electrodes that are formed from distinct combinations of materials. Typically, these systems comprise a plurality of electrodes including a first working electrode comprising a first electrochemically reactive surface formed from an iridium (Ir) composition, wherein the first electrochemically reactive surface generates an electrochemical signal that is assessed by the processor in the presence of glucose; a second working electrode comprising a second electrochemically reactive surface formed from a platinum (Pt) composition, wherein the second electrochemically reactive surface generates an electrochemical signal that is assessed by the processor in the presence of glucose; a counter electrode; and a reference electrode. These systems can further include a computer-readable program code having instructions, which, when executed, cause the processor to: assess electrochemical signal data obtained from the first working electrode and the second working electrode; and then compute a glucose concentration based upon the electrochemical signal data obtained from the first working electrode and/or the second working electrode.

Embodiments of the glucose sensors disclosed herein comprise a first working electrode formed from iridium (for example iridium oxide) and adapted to sense blood glucose concentrations in a specific range, for example between 40-400 mg/dL; and/or between 40-100 mg/dL (e.g. to measure hypoglycemia); and/or between 70-400 mg/dL (e.g. to measure hyperglycemia). Embodiments of the glucose sensors disclosed herein further comprise a second working electrode formed from platinum (for example platinum black) and adapted to sense blood glucose concentrations in a specific range, for example between 40-400 mg/dL; and/or between 40-100 mg/dL (e.g. to measure hypoglycemia); and/or between 70-400 mg/dL (e.g. to measure hyperglycemia). Each of these working electrodes is typically coated with a plurality of layered compositions including, for example, a glucose oxidase composition; and/or an interference rejection composition; and/or a composition that modulates the diffusion of glucose therethrough. Typically, the first and second working electrodes are coated with different layered materials. For example, in some embodiments of the invention, the first working electrode is not coated with an interference rejection layer and the second working electrode is coated with a interference rejection layer. Similarly, in some embodiments of the invention, the first working electrode is coated with a first glucose oxidase layer and the second working electrode is coated with a second glucose oxidase layer, wherein the amount of glucose oxidase in the first glucose oxidase layer is greater than the amount of glucose oxidase in the second glucose oxidase layer. In yet another illustration of working electrodes coated with different layered materials, the first working electrode can be coated with a first glucose modulating layer having a first rate of glucose diffusion and the second working electrode can be coated with a second glucose modulating layer having a second rate of glucose diffusion, wherein the first rate of glucose diffusion is less than the second rate of glucose diffusion.

Embodiments of the invention include those where a surface feature or architecture of an electrode is designed to exhibit certain characteristics. For example, in certain embodiments of the invention, the first and/or second working electrode exhibits an electrochemically reactive surface area that is at least 25% greater than the geometrical surface area of the first working electrode. In yet another embodiment of the invention, the first and/or second working electrodes can be formed from a cylindrical wire having a diameter less than 0.0015 inches. In other embodiments of the invention, the first and/or second working electrodes are formed as planar polygons (e.g. rectangles). Optionally, the size of one of the working electrodes is at least 1.5, 2 or 2.5 folds larger than the size of the other working electrode.

The glucose sensors of the invention are typically coupled to a processor that facilitates the collection, storage and/or analysis of data obtained from the sensor electrodes. In certain embodiments of the invention, the processor evaluates data resulting from a plurality of different voltages applied to the system. In one illustrative embodiment of the invention, the processor evaluates data resulting from a plurality of different voltage pulses applied to the first working electrode that comprises iridium and the second working electrode that comprises platinum. In some embodiments of the invention, the data evaluated results from a voltage potential of between 0.2 and 0.6 volts applied to the first working electrode; and a voltage potential of between 0.5 and 0.7 volts applied to the second working electrode.

In some embodiments of the invention, the processor assesses electrochemical signal data obtained from the first working electrode and the second working electrode against one or more reliability parameters; ranks the electrochemical signal data obtained from the first working electrode and the second working electrode; and then computes a glucose concentration based upon the ranking of electrochemical signal data obtained from the first working electrode and the second working electrode. In certain embodiments of the invention, the processor compares the electrochemical signal data from the first working electrode and the second working electrode in order to obtain information that, for example, provides an indication on how one or more electrochemical signals from the first working electrode or the second working electrode correlates with actual glucose blood concentrations in a diabetic patient. In typical embodiments of the invention, the comparison includes observing whether a signal obtained from the first working electrode and/or the second working electrode falls within a predetermined range of values. In other embodiments of the invention, the comparison includes observing a trend in sensor signal data from the first working electrode and/or the second working electrode. In yet other embodiments of the invention, the comparison includes observing an amount of nonspecific signal noise in the first working electrode and/or the second working electrode. Using embodiments of the invention disclosed herein, one can identify one or more signals observed by the sensor that is indicative of increasing glucose blood concentrations or decreasing blood glucose concentrations in the diabetic patient and/or is indicative of the presence of interfering compounds; and/or is indicative of background noise; and/or is indicative of sensor hydration; and/or is indicative of sensor signal drift; and/or is indicative of sensor loss of sensitivity to glucose.

Certain glucose sensor system embodiments of the invention are combined with additional elements to facilitate their use in various contexts, for example a monitor adapted to display discreet signal information from the first working electrode and/or the second working electrode. Other embodiments of the invention comprise a probe that is adapted to be inserted in vivo and includes an electrode array comprising the first working electrode, the second working electrode, the counter electrode, and the reference electrode. Some embodiments of the invention include multiple probes with discreet electrode arrays that are configured to be electronically independent of each other. Optionally the probes are coupled to a probe platform. In certain embodiments of the invention, first and second probes are oriented on the probe platform so that the first and second electrode arrays are located at different depths when inserted into an in vivo environment. In other embodiments of the invention, a first probe and a second probe are coupled to the probe platform and the probe platform is made from a flexible material that allows the probes to twist and bend when implanted in vivo in a manner that inhibits in vivo movement of the probes. Other embodiments of the invention comprise an adhesive patch adapted to secure the probe(s) and/or the probe platform to the skin of a diabetic patient.

Other embodiments of the invention include methods for computing blood glucose concentrations in a diabetic patient. In typical embodiments of the invention, the method comprises observing electrochemical signal data generated by the sensor systems disclosed herein (e.g. those that include multiple working electrodes having different material properties as well as the algorithms designed for use with such systems). In typical embodiments of the invention, the method comprises comparing the electrochemical signal data from the first working electrode and the second working electrode; and then computing blood glucose concentration using the comparison of the electrochemical signal data obtained from the first working electrode and the second working electrode. Optionally in such methods, the comparison of electrochemical signal data from the first working electrode and the second working electrode includes observing whether a signal obtained from the first working electrode and the second working electrode falls within a predetermined range of values; observing a trend in sensor signal data from the first working electrode and the second working electrode; or observing an amount of nonspecific signal noise in the first working electrode and the second working electrode.

Yet another embodiment of the invention is a composition of matter comprising an iridium composition having an electrochemically reactive surface; a glucose oxidase composition disposed upon the electrochemically reactive surface; and an analyte modulating layer disposed upon the glucose oxidase composition. In such embodiments of the invention, the analyte modulating layer typically comprises a linear polyurethane/polyurea polymer; a branched acrylate polymer; or a blended mixture of the linear polyurethane/polyurea polymer and the branched acrylate polymer, wherein the mixture is blended at a ratio of between 1:1 and 1:20 by weight percentage. In one illustrative embodiment of this composition, the linear polyurethane/polyurea polymer is formed from a mixture comprising: a diisocyanate; at least one hydrophilic diol or hydrophilic diamine; and a siloxane. In another illustrative embodiment of this composition, the branched acrylate polymer is formed from a mixture comprising a 2-(dimethylamino)ethyl methacrylate; a methyl methacrylate; a polydimethyl siloxane monomethacryloxypropyl; and a poly(ethylene oxide) methyl ether methacrylate.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE FIGURES

FIG. 1 provides a schematic of the well known reaction between glucose and glucose oxidase. As shown in a stepwise manner, this reaction involves glucose oxidase (GOx), glucose and oxygen in water. In the reductive half of the reaction, two protons and electrons are transferred from β-D-glucose to the enzyme yielding d-gluconolactone. In the oxidative half of the reaction, the enzyme is oxidized by molecular oxygen yielding hydrogen peroxide. The d-gluconolactone then reacts with water to hydrolyze the lactone ring and produce gluconic acid. In certain electrochemical sensors of the invention, the hydrogen peroxide produced by this reaction is oxidized at the working electrode (H₂O₂→2H⁺+O₂+2e⁻).

FIG. 2A provides a diagrammatic view of one embodiment of an amperometric analyte sensor to which an interference rejection membrane can be added. FIG. 2B provides a diagrammatic view of one embodiment of an amperometric analyte sensor having an interference rejection membrane. FIG. 2C provides a diagrammatic view of a specific embodiment of an amperometric glucose sensor having a plurality of layers including a layer of a glucose limiting membrane (GLM), a layer of an adhesion promoter, a layer of human serum albumin (HSA), a layer of glucose oxidase, a layer of an interference rejection membrane (IRM), and an electrode layer, all of which are supported by a base comprised of a polyimide composition.

FIGS. 3A-3L provide data showing various characteristics of electrodes and electrode materials that are useful in embodiments of the invention. In FIGS. 3A-3K, the disclosure in the first drawings figure (e.g. “FIG. 3A-1, FIG. 3B-1” etc.) shows a scanning electron microscope (SEM) image of the surface of the electrode and specific features of this electrode such as geometric surface area, surface area ratio, roughness characteristics such as Ravg and/or Rmax, and impedance; the data in the second drawings figure (e.g. “FIG. 3A-2, FIG. 3B-2” etc.) shows a graph of cyclic voltammetry characteristics of the electrode; and the data in the third drawings figure (e.g. “FIG. 3A-3, FIG. 3B-3” etc.) shows a graph of linearity characteristics of the electrode material. FIG. 3A shows data from an electrode formed by electroplating platinum black; FIG. 3B shows data from an electrode formed from a Pt wire; FIG. 3C shows data from an electrode formed from a Pt coated Pt wire; FIG. 3D shows data from an electrode formed from a Pt/Ir 80/20 wire; FIG. 3E shows data from an electrode formed from a Pt coated Pt/Ir 80/20 wire; FIG. 3F shows data from an electrode formed from a Pt coated flexible substrate (50 nm); FIG. 3G shows data from an electrode formed from a Pt coated flexible substrate (500 nm); FIG. 3H shows data from an electrode formed from an Ir coated flexible substrate (50 nm); FIG. 3I shows data from an electrode formed from an Ir coated flexible substrate (500 nm); FIG. 3J shows data from an electrode formed from an IrOx coated flexible substrate (50 nm); and FIG. 3K shows data from an electrode formed from an IrOx coated flexible substrate (500 nm), FIG. 3L shows data illustrating the base metal mechanical properties of certain electrode compositions.

FIG. 4 provides a chart showing selected characteristics of Iridium and IrO₂ electrodes.

FIGS. 5A-5D provide data showing various characteristics of Iridium and Iridium oxide electrodes. FIG. 5A shows graphic cyclic voltammetry data from Ir (left panel) and IrO₂ (right panel) electrodes. FIG. 5B shows the different signals that are generated by CS nominal electrodes formed from Pt black (left panel) as compared to electrodes formed from IrO₂ (right panel) in the presence of the interfering compounds acetaminophen and ascorbic acid. FIG. 5C shows in vitro and in vivo data generated by sensors formed from either Iridium (upper panel) or Iridium Oxide (lower panel). FIG. 5D shows in vivo data generated by sensors formed from Iridium Oxide in diabetic (left panel) and non-diabetic (right panel) dogs.

FIG. 6 shows an illustrative embodiment of a sensor probe arrangement. In this embodiment, each sensor probe has 2 electrode arrays. In this embodiment, each electrode array is a 3 electrode system with a working (e.g. one formed from either a Pt or an Ir composition), counter, and reference electrode so that the assembly includes 4 electrode arrays on 2 sensor probes. In this embodiment, the 4 independent glucose sensor signals allows for improved system reliability and accuracy, factors which can be further enhanced for example through the use of certain algorithms disclosed herein.

FIG. 7 presents an exemplary generalized computer system 202 that can be used to implement elements of the present invention.

FIGS. 8A and 8B shows illustrative embodiments of and provides comments on ribbon wire (FIG. 8A) and coiled wire (FIG. 8B) substrates that can be used with embodiments of the invention.

### DETAILED DESCRIPTION OF THE EMBODIMENTS

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.

It must be noted that as used herein and in the appended claims, the singular forms “a”, “and”, and “the” include plural referents unless the context clearly dictates otherwise. Thus, for example, reference to “an electrode” includes a plurality of electrodes, and so forth. All numbers recited in the specification and associated claims that refer to values that can be numerically characterized with a value other than a whole number (e.g. the concentration of a compound in a solution) are understood to be modified by the term “about”.

The term “analyte” as used herein is a broad term and is used in its ordinary sense, including, without limitation, to refer to a substance or chemical constituent in a fluid such as a biological fluid (for example, blood, interstitial fluid, cerebral spinal fluid, lymph fluid or urine) that can be analyzed. Analytes can include naturally occurring substances, artificial substances, metabolites, and/or reaction products. In the typical embodiments of the invention that are disclosed herein, the analyte is glucose. Salts, sugars, proteins fats, vitamins and hormones naturally occurring in blood or interstitial fluids can constitute analytes in certain embodiments. The analyte can be naturally present in the biological fluid or endogenous; for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin. The metabolic products of drugs and pharmaceutical compositions are also contemplated analytes.

The terms “interferents” and “interfering species/compounds” are used in accordance with their art accepted meaning, including, but not limited to, effects and/or chemical species/compounds that interfere with the measurement of an analyte of interest in a sensor to produce a signal that does not accurately represent the analyte measurement. In one example of an electrochemical sensor, interfering species are compounds with an oxidation potential that overlaps with the analyte to be measured so as to produce spurious signals.

The terms “electrochemically reactive surface” and “electroactive surface” as used herein are broad terms and are used in their ordinary sense, including, without limitation, the surface of an electrode where an electrochemical reaction takes place. In one example, a working electrode (e.g. one comprised of iridium oxide or platinum black) measures hydrogen peroxide produced by the enzyme catalyzed reaction of the analyte being detected reacts creating an electric current (for example, detection of glucose analyte utilizing glucose oxidase produces H₂O₂ as a byproduct, H₂O₂ reacts with the surface of the working electrode producing two protons (2H⁺), two electrons (2e⁻) and one molecule of oxygen (O₂) which produces the electronic current being detected). In the case of the counter electrode, a reducible species, for example, O₂ is reduced at the electrode surface in order to balance the current being generated by the working electrode.

As discussed in detail below, embodiments of the invention relate to the use of an electrochemical sensor that exhibits a novel constellation of elements including multiple working electrodes having different material properties as well as algorithms for use with such sensors, constellations of elements that provide a unique set of technically desirable properties. In some embodiments, the sensor is a continuous device, for example a subcutaneous, transdermal, or intravascular device. In some embodiments, the device can analyze a plurality of intermittent blood samples. Typically, the sensor is of the type that senses a product or reactant of an enzymatic reaction between an analyte and an enzyme in the presence of oxygen as a measure of the analyte in vivo or in vitro. Such sensors typically comprise a diffusion modulating membrane surrounding the enzyme through which an analyte migrates. The product is then measured using electrochemical methods and thus data obtained from the electrodes within the system functions to provide a measure of the analyte.

Embodiments of the invention disclosed herein provide sensors of the type used, for example, in subcutaneous or transcutaneous monitoring of blood glucose levels in a diabetic patient. A variety of implantable, electrochemical biosensors have been developed for the treatment of diabetes and other life-threatening diseases. Many existing sensor designs use some form of immobilized enzyme to achieve their bio-specificity. Embodiments of the invention described herein can be adapted and implemented with a wide variety of known electrochemical sensors, including for example, U.S. Patent Application No. 20050115832, U.S. Pat. Nos. 6,001,067, 6,702,857, 6,212,416, 6,119,028, 6,400,974, 6,595,919, 6,141,573, 6,122,536, 6,512,939 5,605,152, 4,431,004, 4,703,756, 6,514,718, 5,985,129, 5,390,691, 5,391, 250, 5,482,473, 5,299,571, 5,568,806, 5,494,562, 6,120,676, 6,542,765 as well as PCT International Publication Numbers WO 01/58348, WO 04/021877, WO 03/034902, WO 03/035117, WO 03/035891, WO 03/023388, WO 03/022128, WO 03/022352, WO 03/023708, WO 03/036255, WO03/036310 WO 08/042,625, and WO 03/074107, and European Patent Application EP 1153571, the contents of each of which are incorporated herein by reference.

As discussed in detail below, embodiments of the invention disclosed herein provide sensor elements having enhanced material properties and/or architectural configurations and sensor systems constructed to include such elements (e.g. those comprising multiple working electrodes having different material properties, associated software and electronic components such as a monitor, a processor and the like). The disclosure further provides methods for making and using such sensors. While typical embodiments of the invention pertain to glucose sensors, a variety of the elements disclosed herein (e.g. the algorithms) can be adapted for use with any one of the wide variety of sensors known in the art. The analyte sensor elements, architectures and methods for making and using these elements that are disclosed herein can be used to establish a variety of layered sensor structures. Such sensors of the invention exhibit a surprising degree of flexibility and versatility, characteristics which allow a wide variety of sensor configurations to be used to examine analytes of interest.

Specific aspects of embodiments of the invention are discussed in detail in the following sections.

### I. Typical Elements, Configurations and Analyte Sensor Embodiments of the Invention

A wide variety of sensors and sensor elements are known in the art including amperometric sensors used to detect and/or measure biological analytes such as glucose. Many glucose sensors are based on an oxygen (Clark-type) amperometric transducer (see, e.g. Yang et al., Electroanalysis 1997, 9, No. 16: 1252-1256; Clark et al., Ann. N.Y. Acad. Sci. 1962, 102, 29; Updike et al., Nature 1967, 214,986; and Wilkins et al., Med. Engin. Physics, 1996, 18, 273.3-51). A number of in vivo glucose sensors utilize hydrogen peroxide-based amperometric transducers because such transducers are relatively easy to fabricate and can readily be miniaturized using conventional technology. However, problems associated with the use of hydrogen peroxide-based amperometric transducers, can include for example, difficulties in maintaining an optimized stoichiometry of the chemical reactants that generate hydrogen peroxide in the presence of analyte, difficulties in measuring a broad range of analyte concentrations, as well as difficulties that result from signal drift and signal interference due to electroactive substances present in the analyte environment. For example, with amperometric glucose sensors that utilize the chemical reaction between glucose and glucose oxidase to generate a measurable signal, these sensors can experience what is known in the art as the “oxygen deficit problem”. Specifically, because glucose oxidase based sensors require both oxygen (O₂) as well as glucose to generate a signal, the presence of an excess of oxygen relative to glucose, is necessary for the operation of a glucose oxidase based glucose sensor. However, because the concentration of oxygen in subcutaneous tissue is much less than that of glucose, oxygen can be the limiting reactant in the reaction between glucose, oxygen, and glucose oxidase in a sensor, a situation which compromises the sensor's ability to produce a signal that is strictly dependent on the concentration of glucose. In addition, with many conventional electrodes, the amperometric measurement of hydrogen peroxide requires an applied potential of 600-700 mV. In this context many endogenous reducing species such as ascorbic acid and urate and some drugs such as acetaminophen (paraqcetamol) will also be oxidized at the electrode, leading to a confounding signal that is not related to the amount of target analyte that the sensor is designed to measure. As discussed in detail below, these and other problems can be avoided by using embodiments of the invention that are disclosed herein.

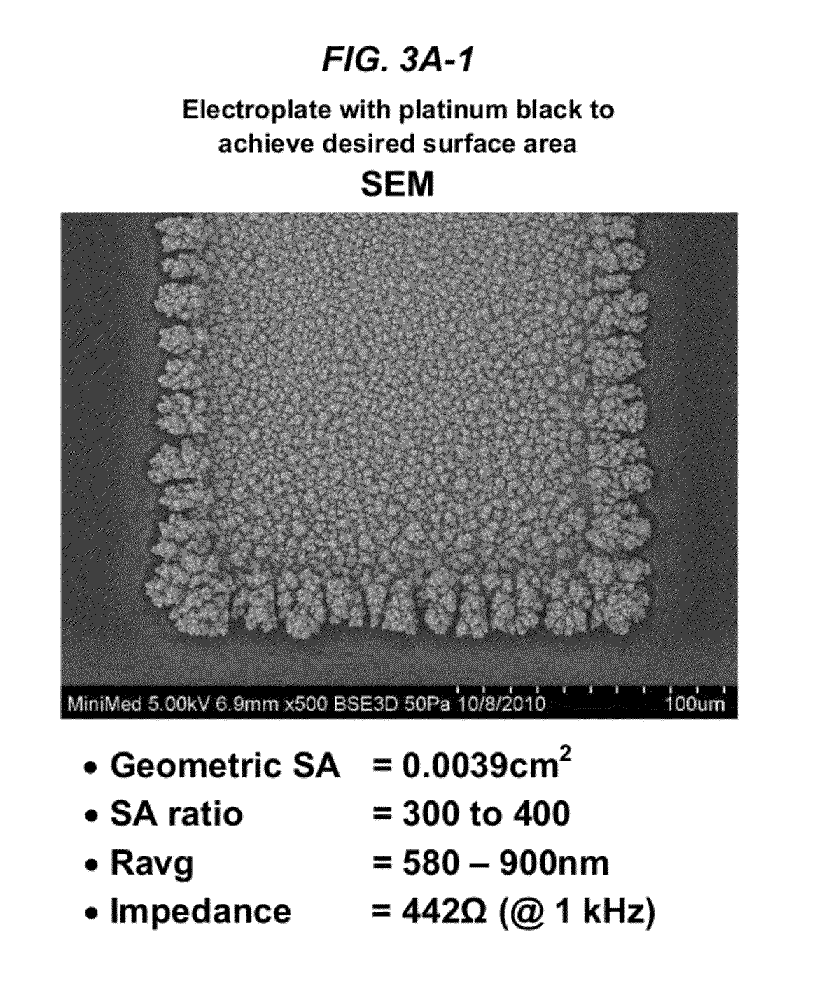

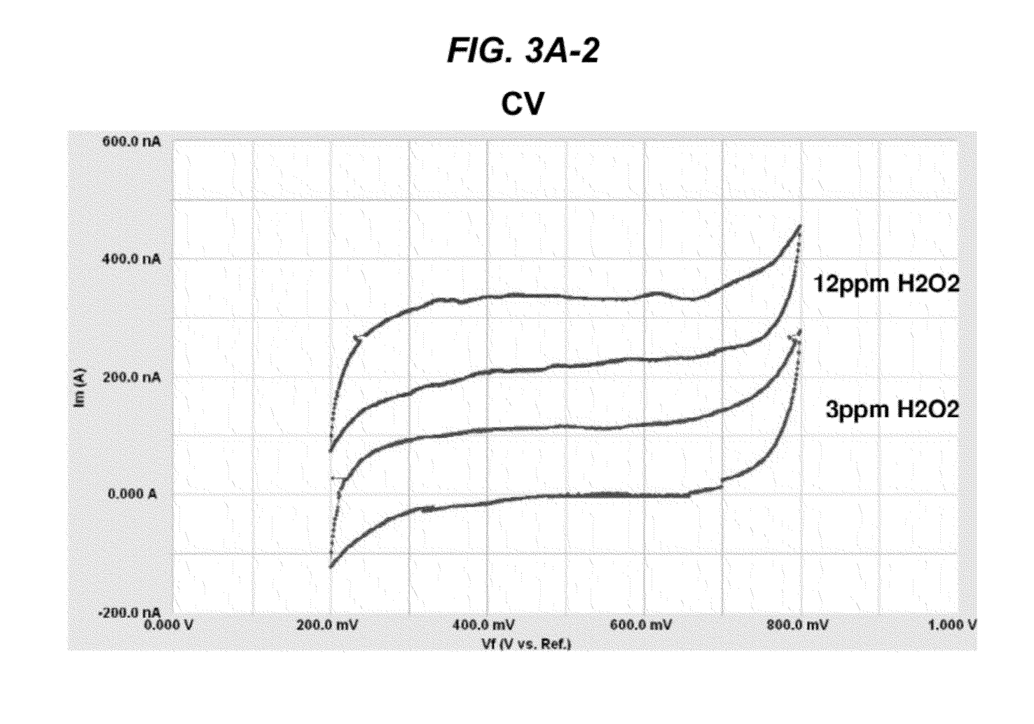

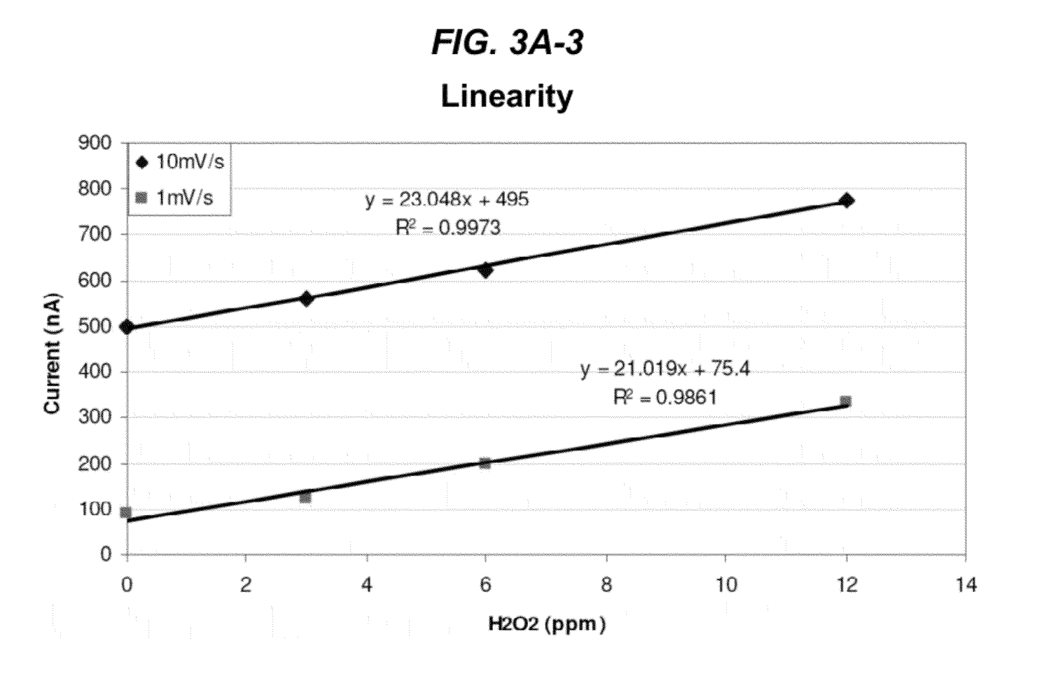

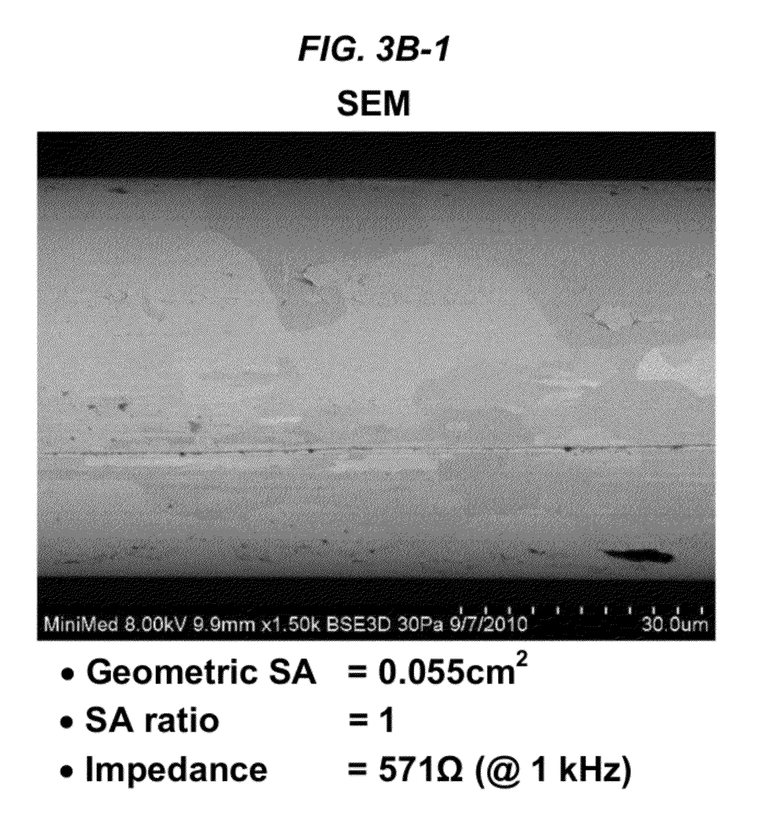

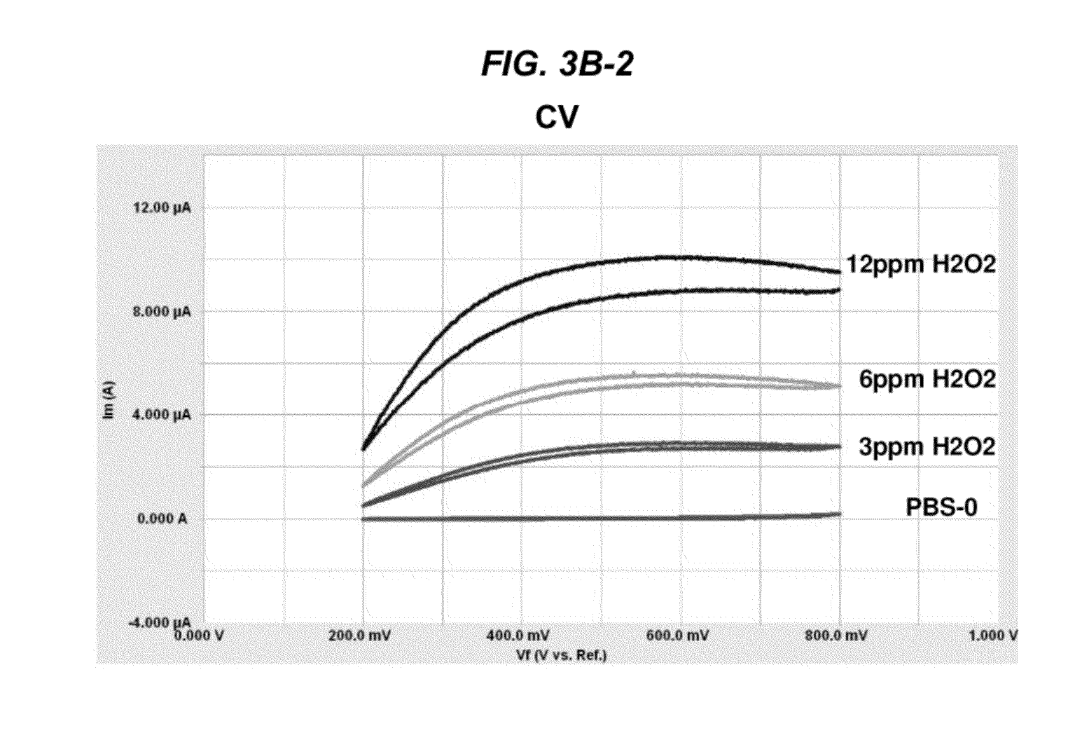

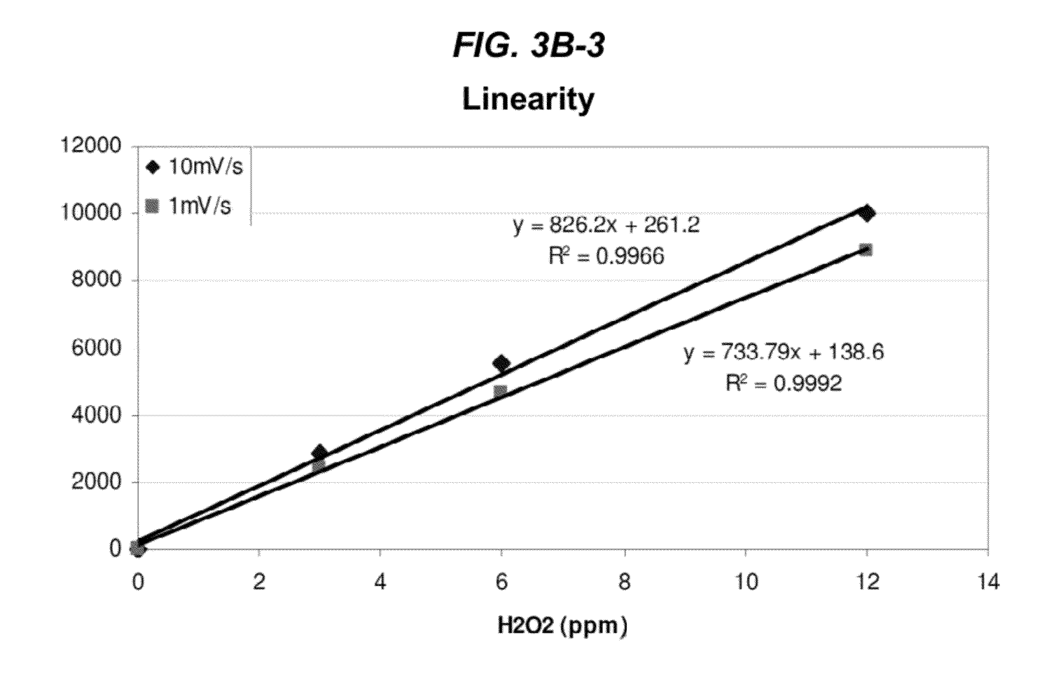

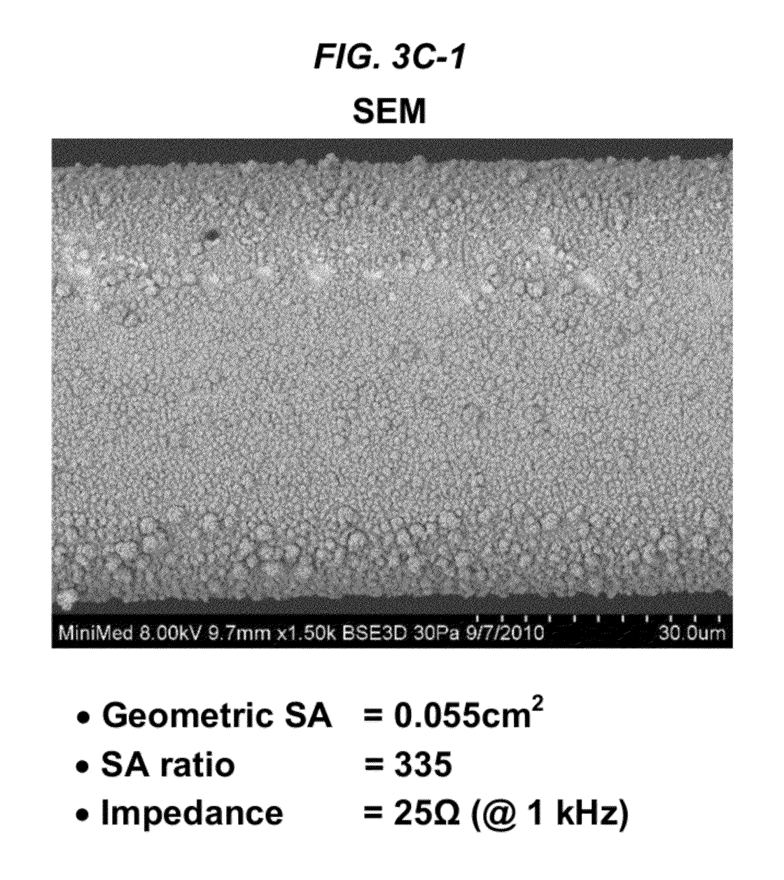

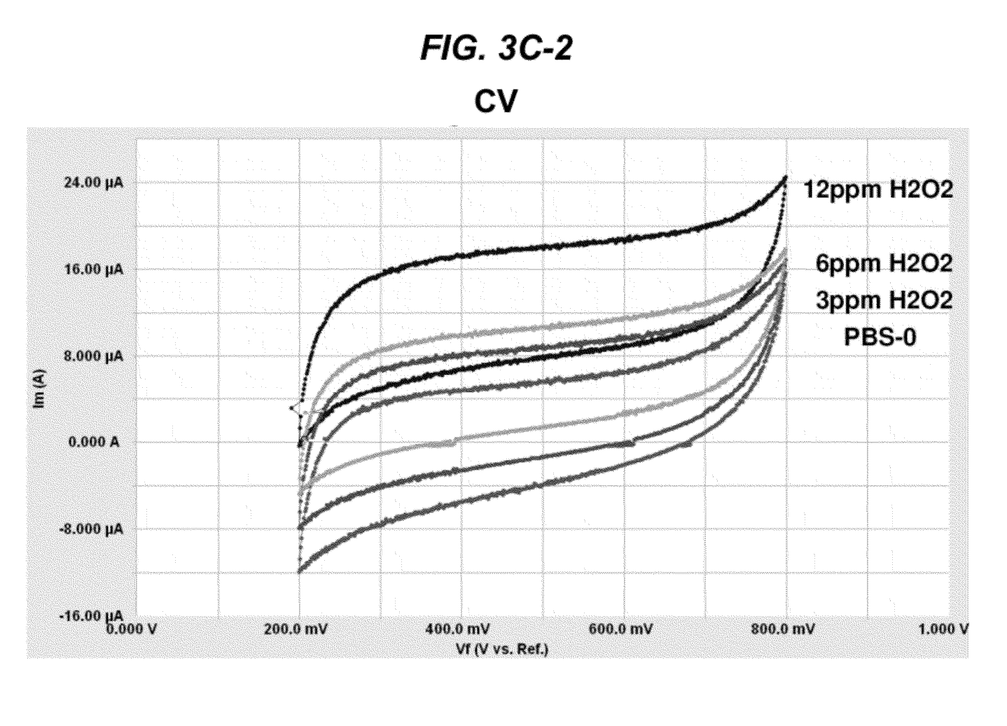

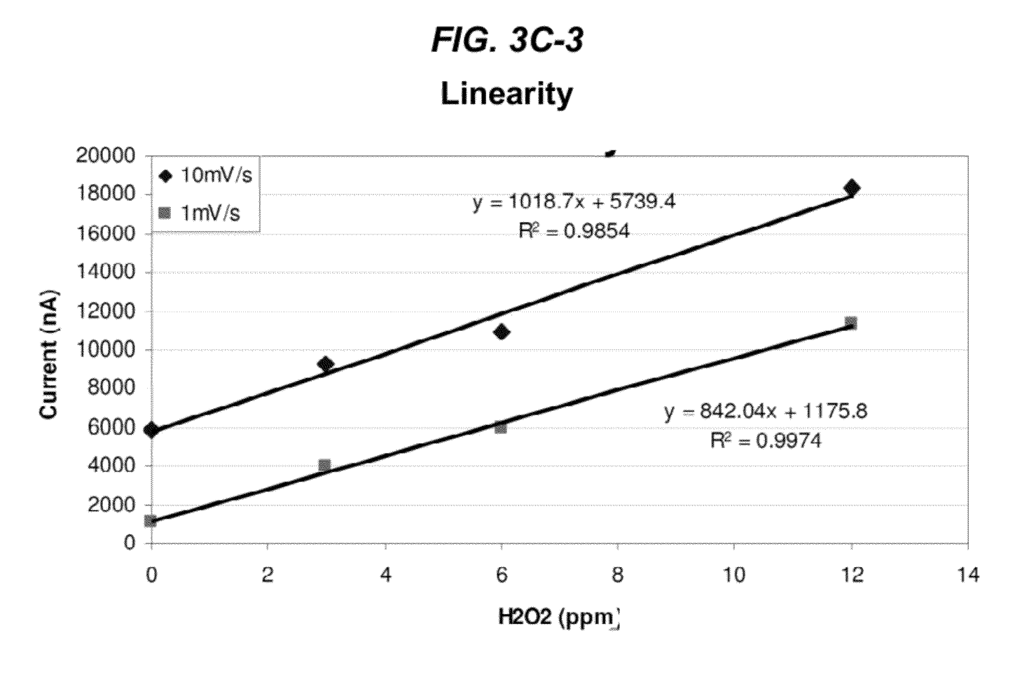

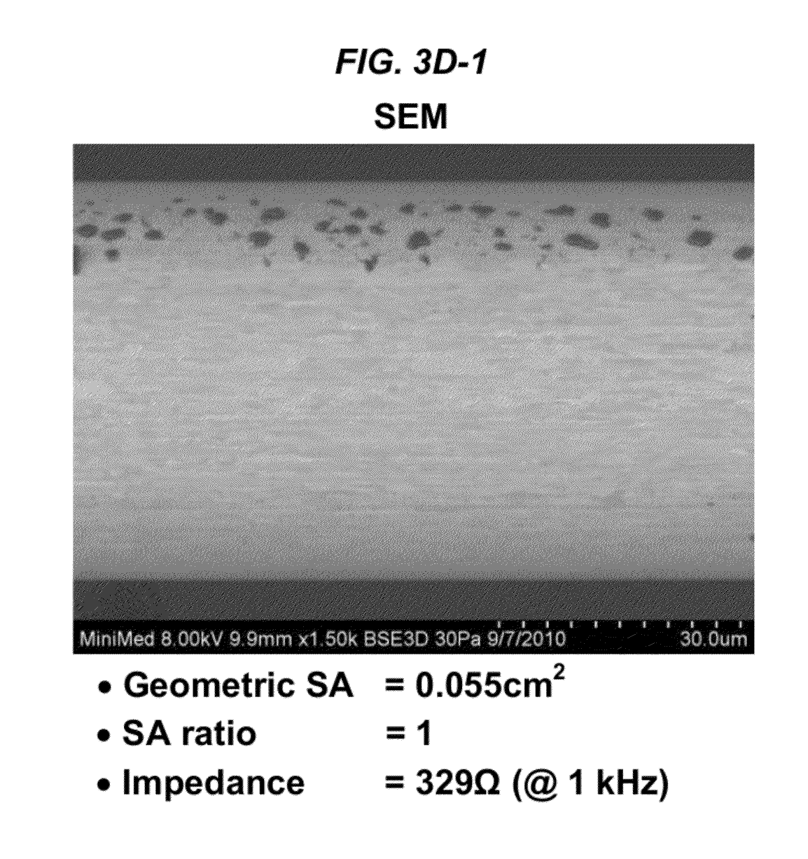

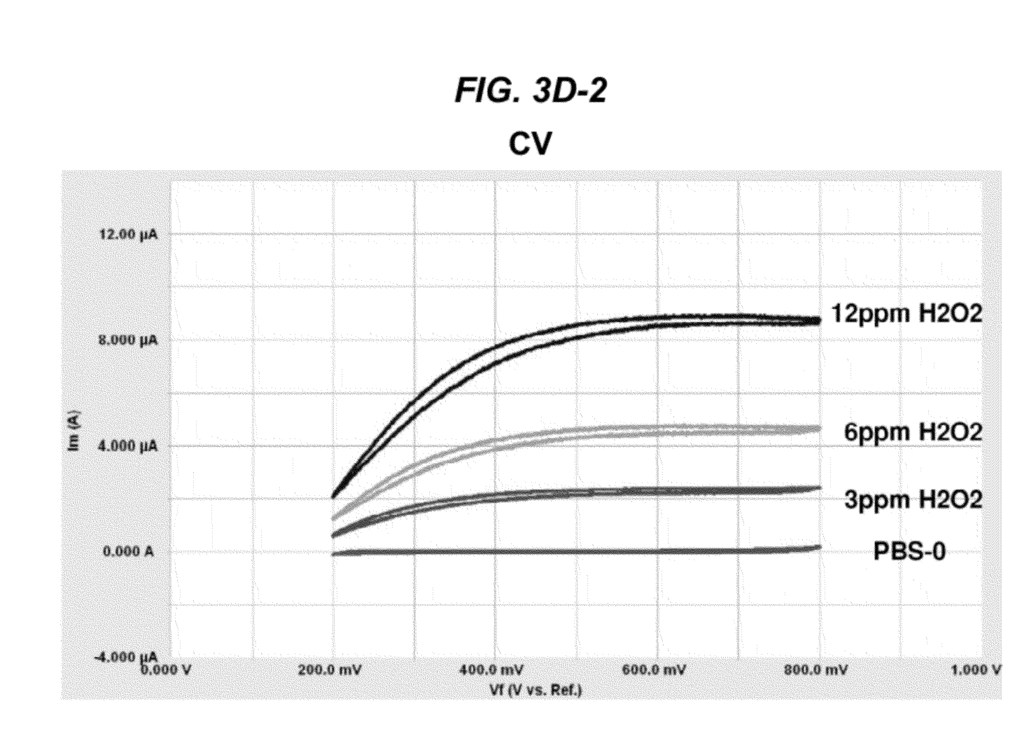

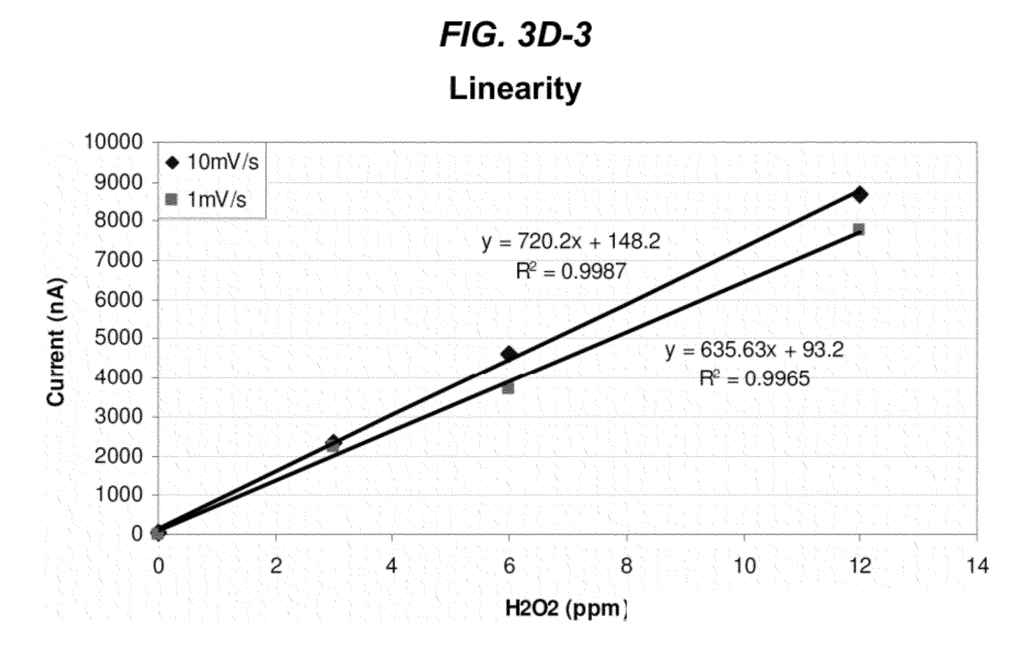

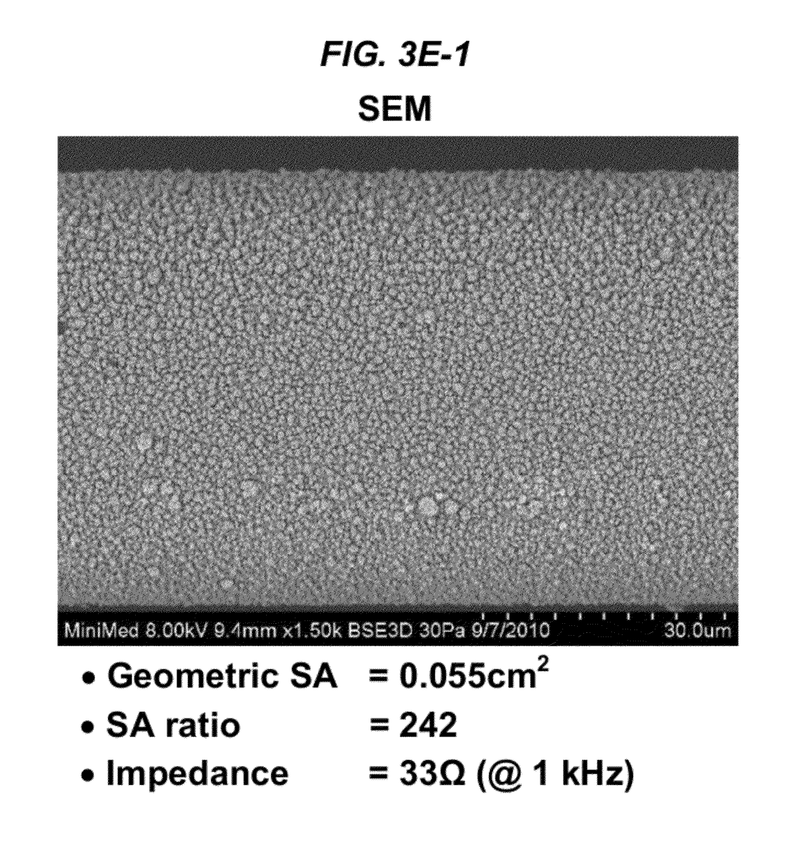

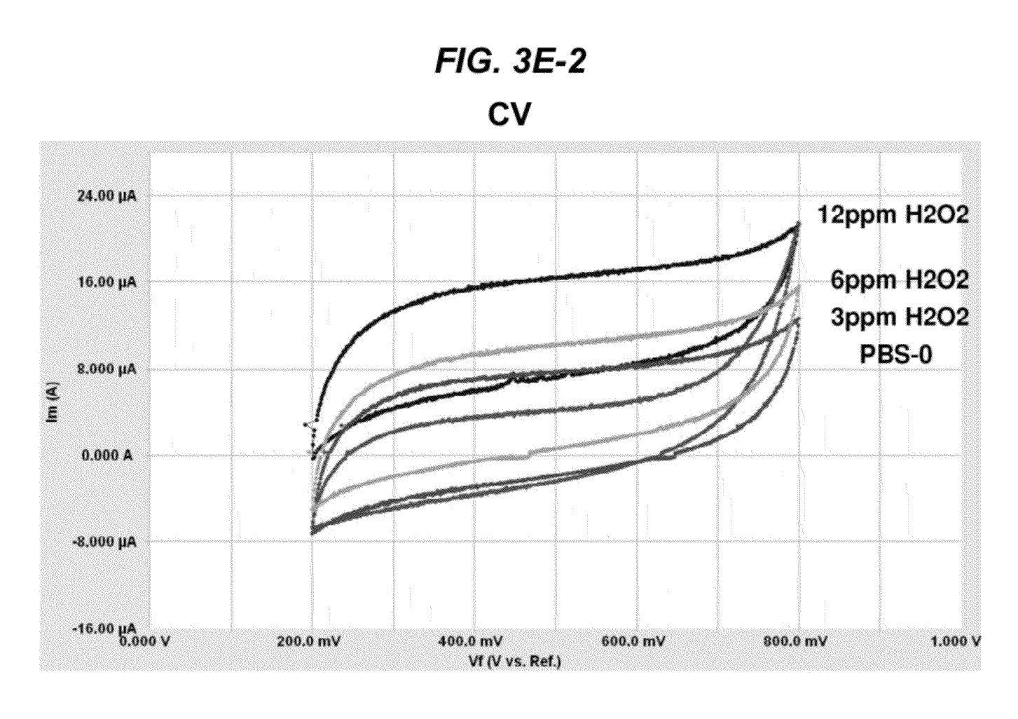

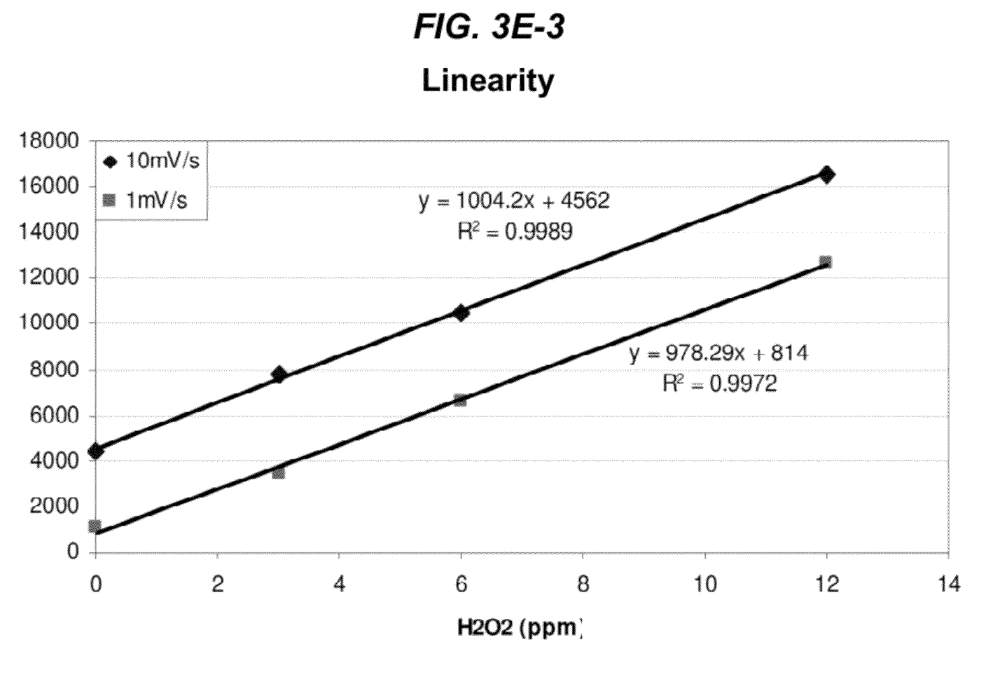

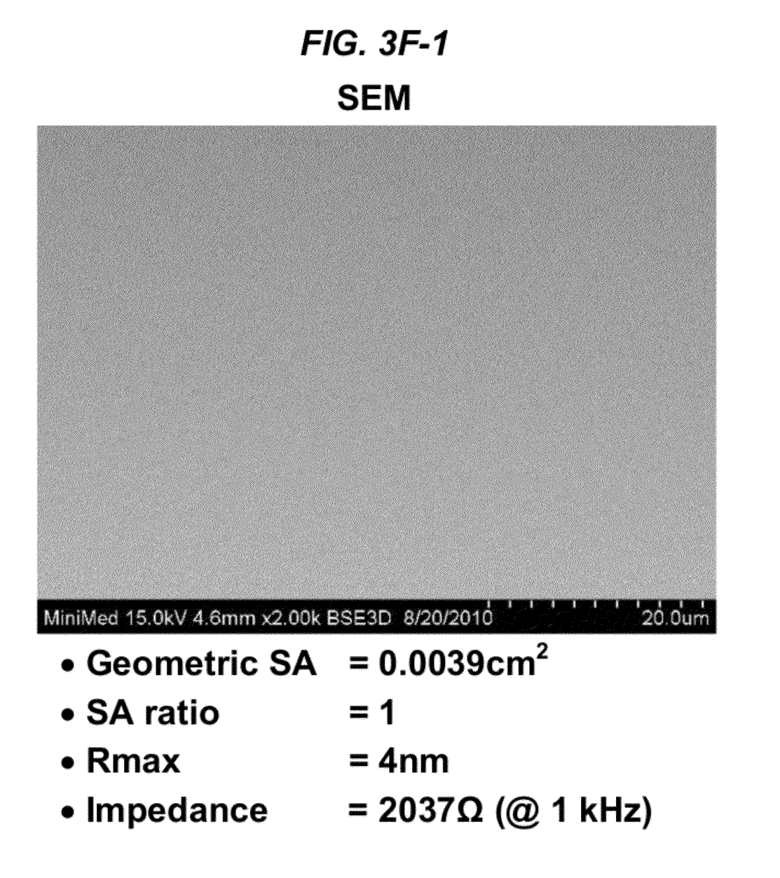

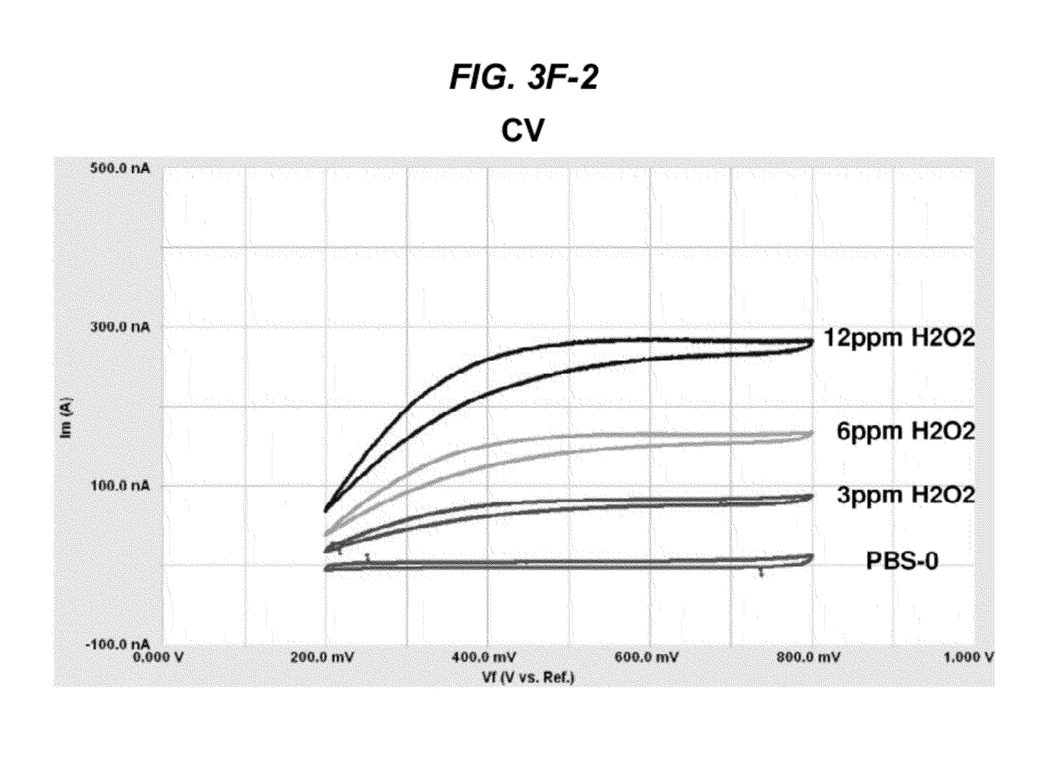

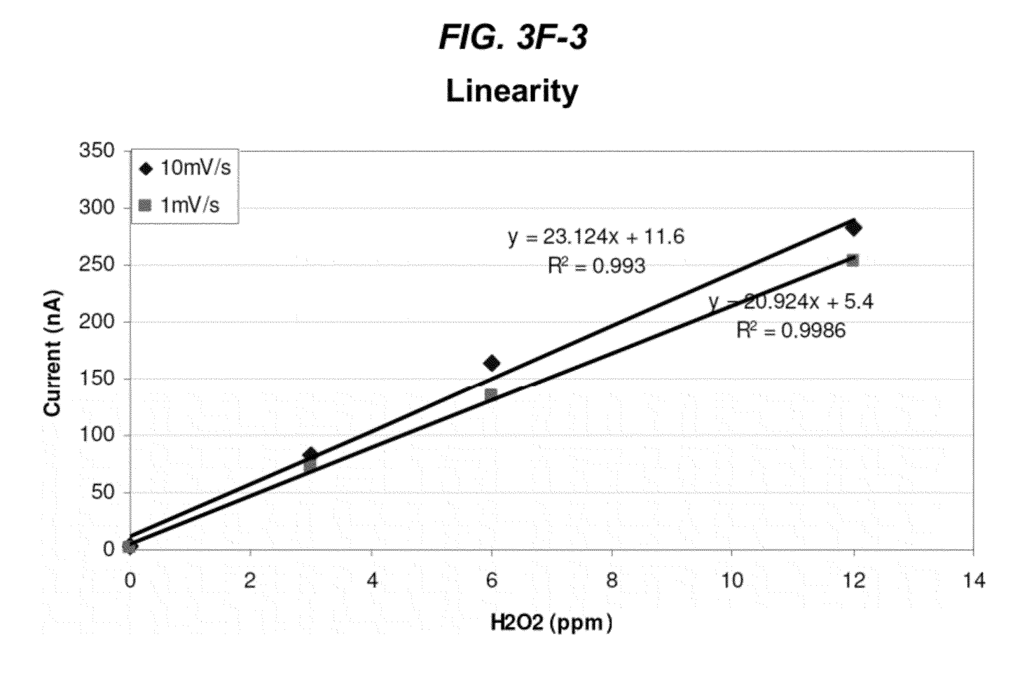

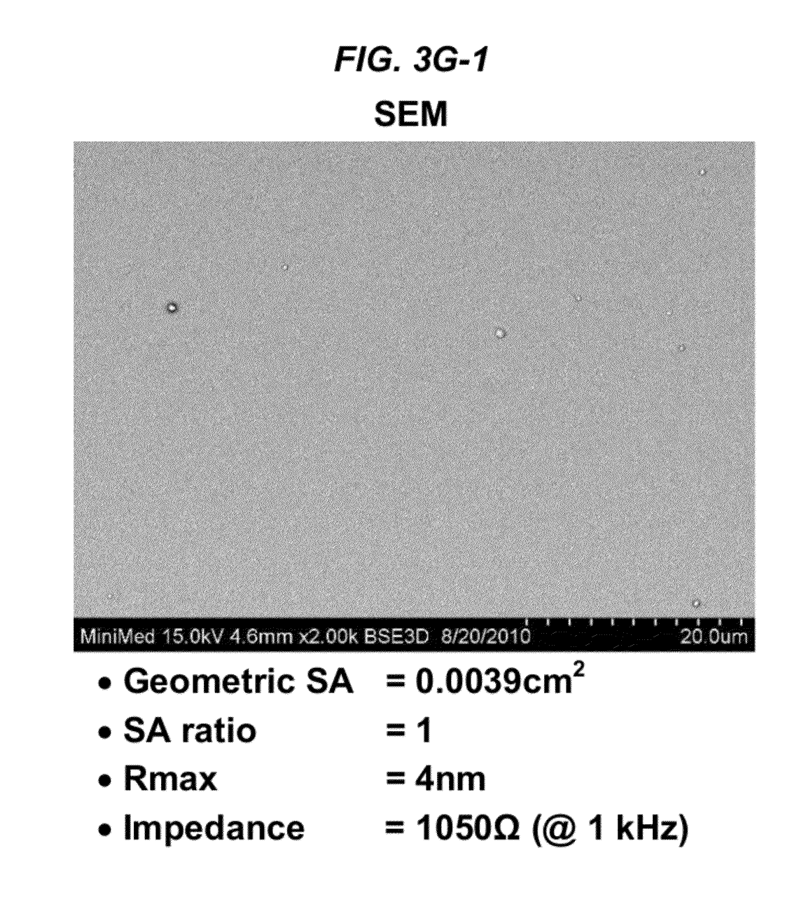

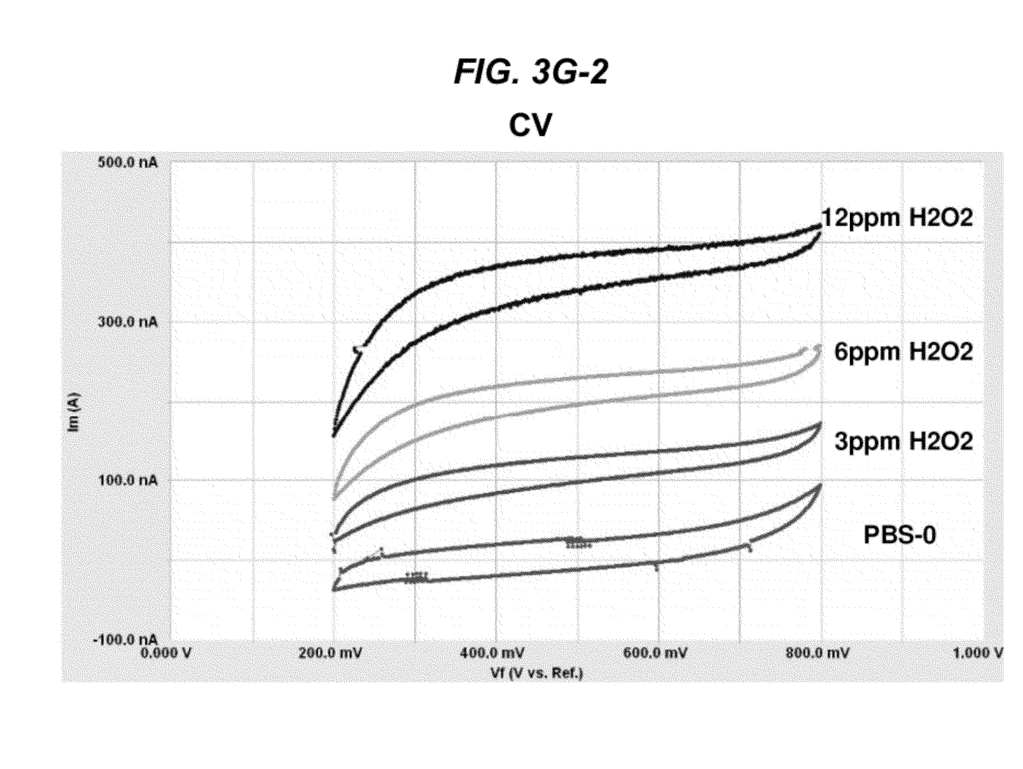

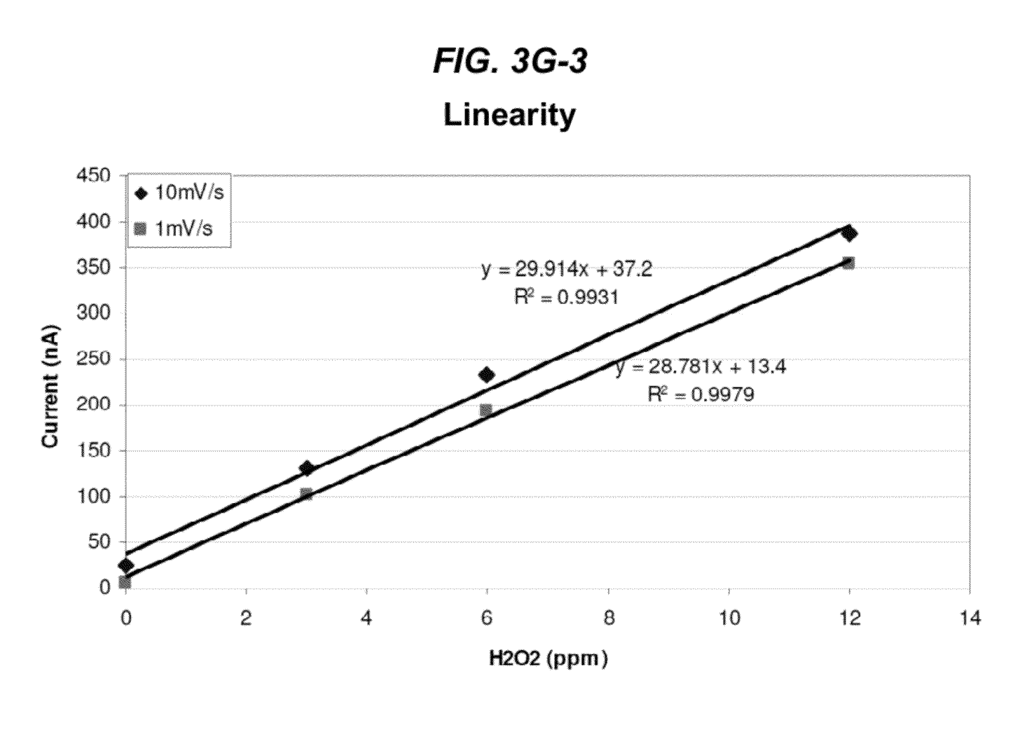

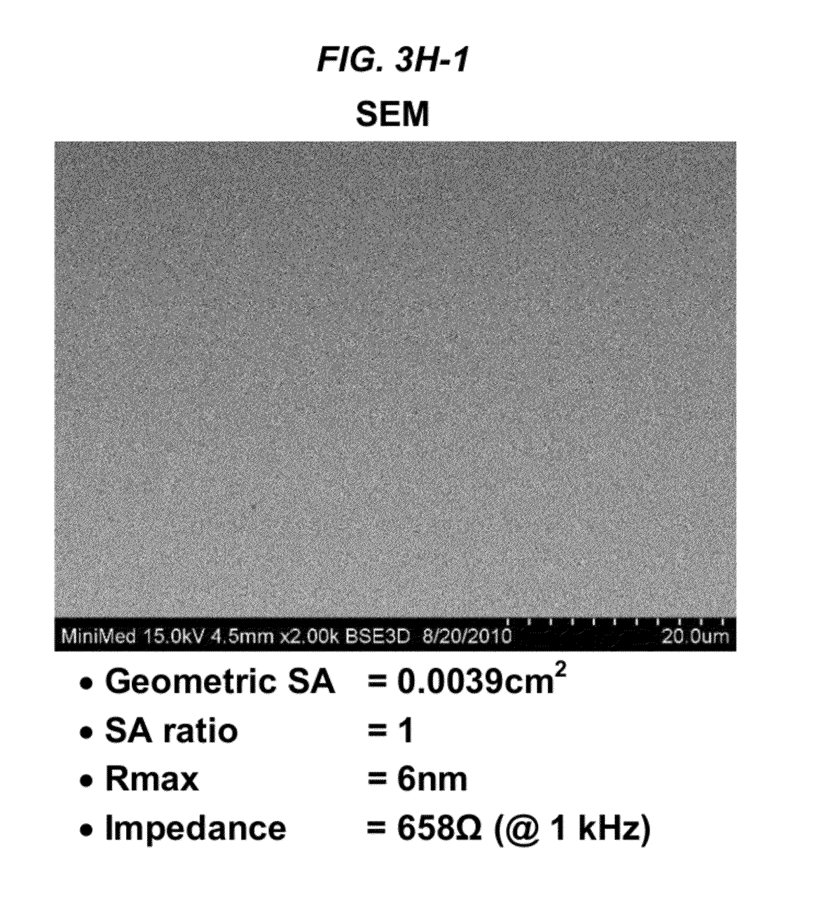

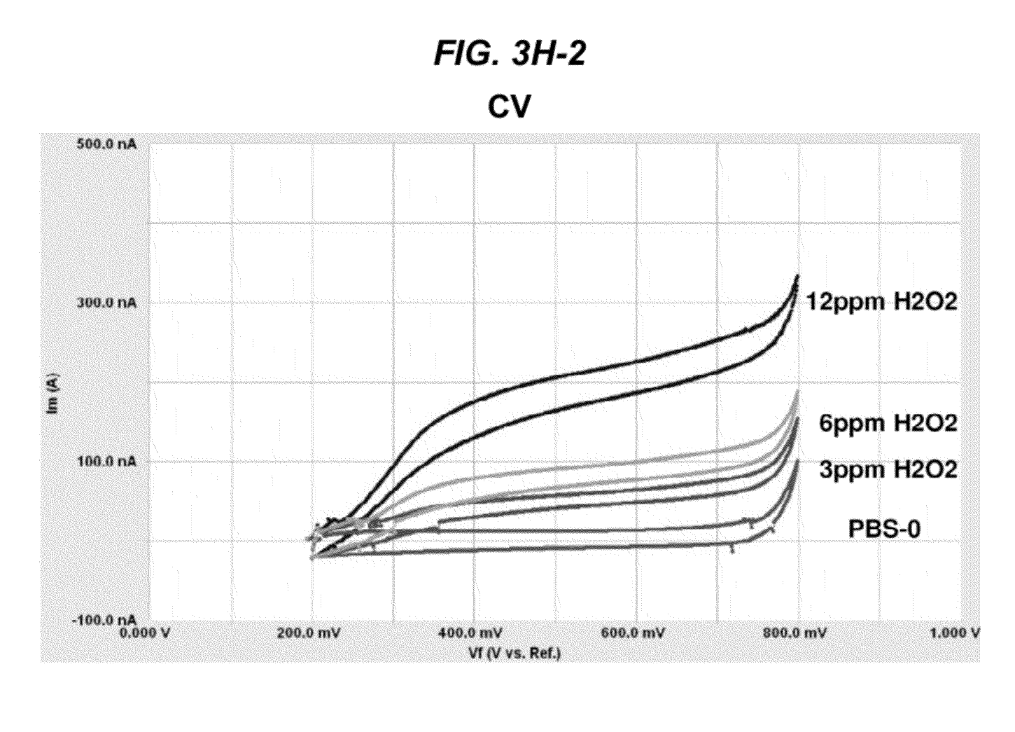

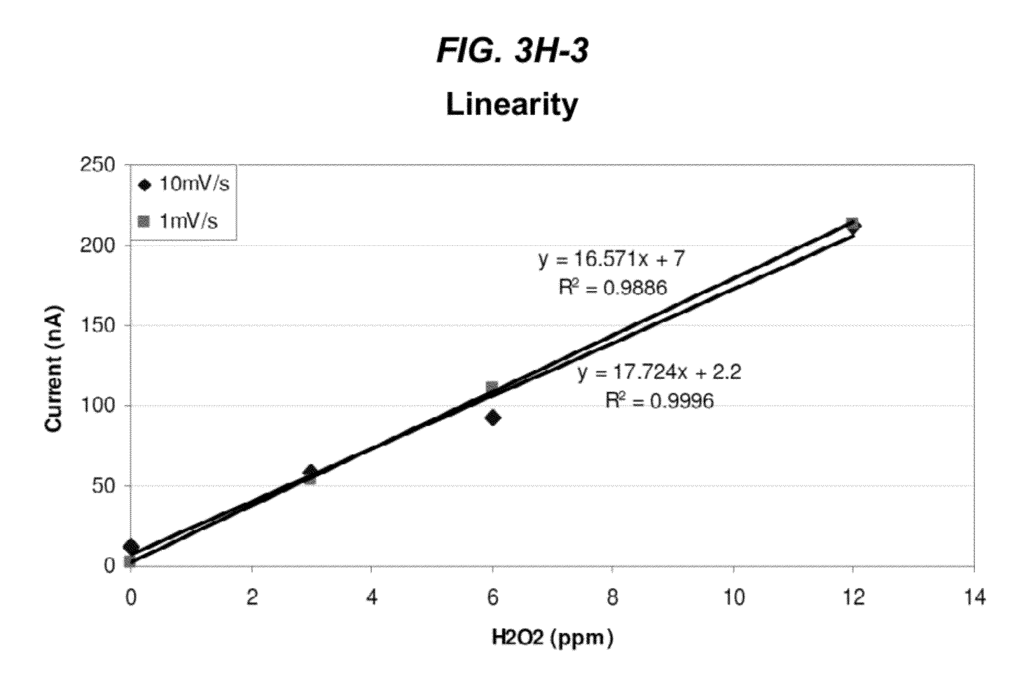

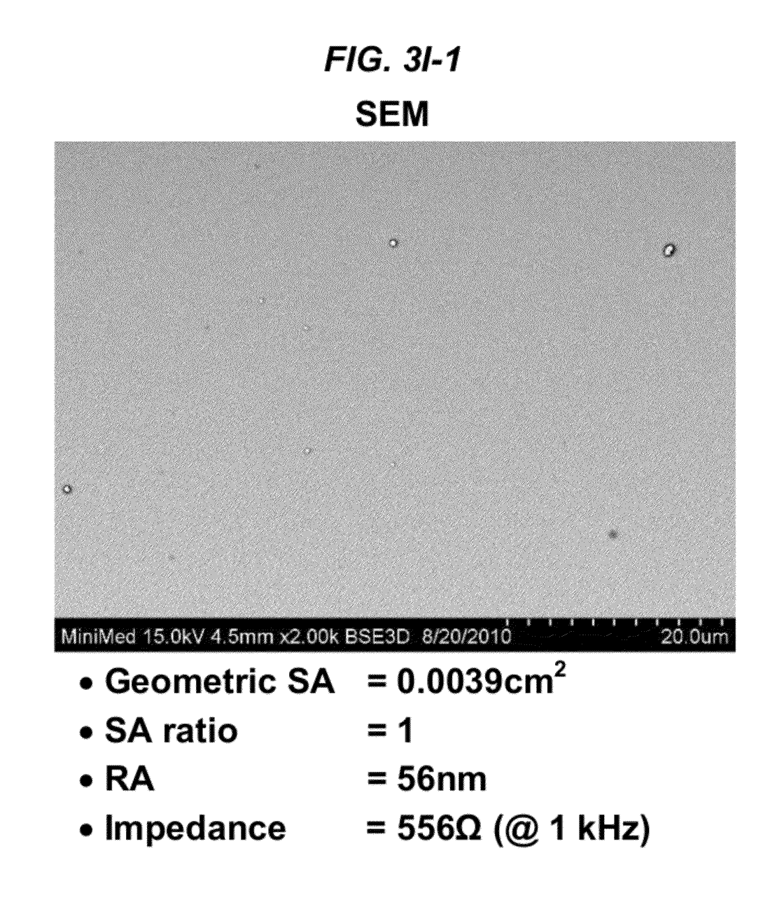

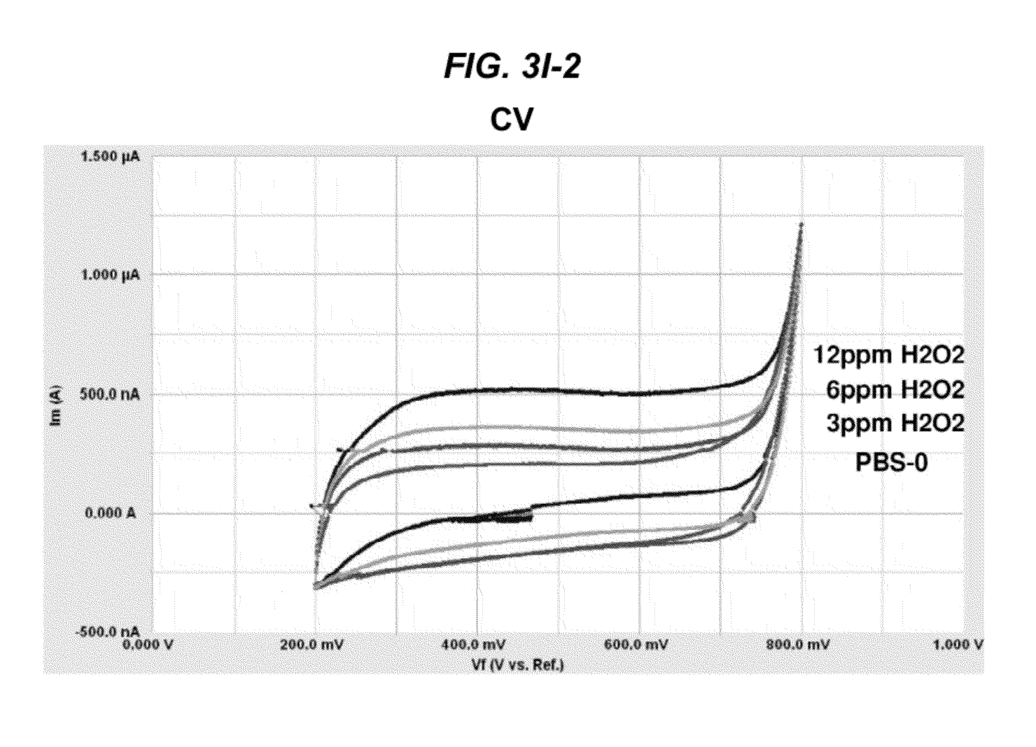

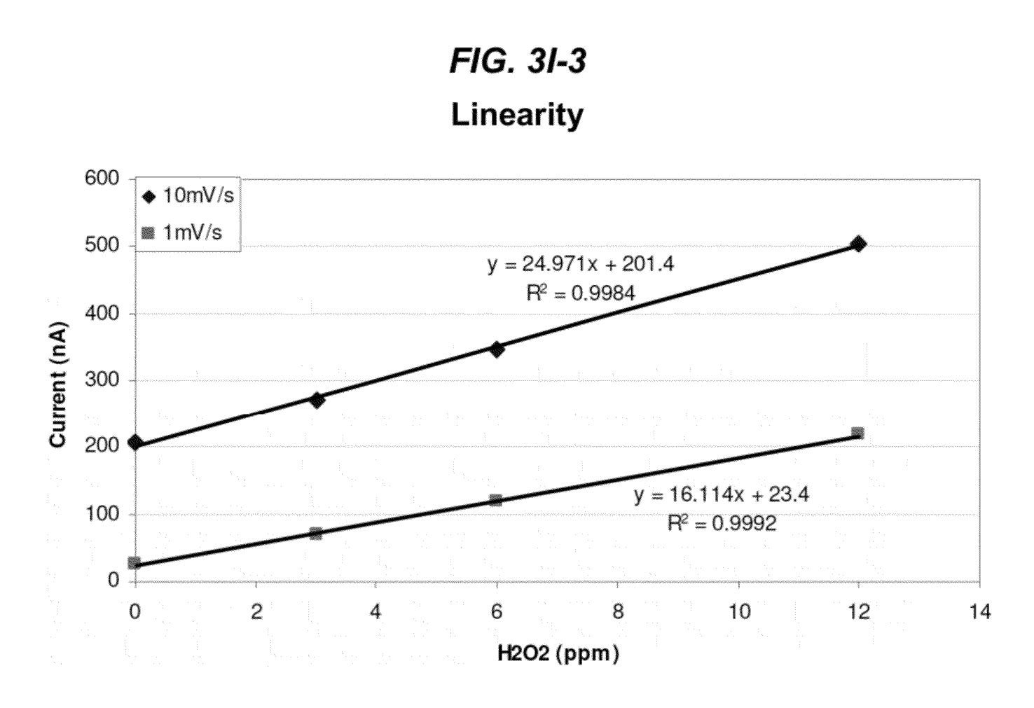

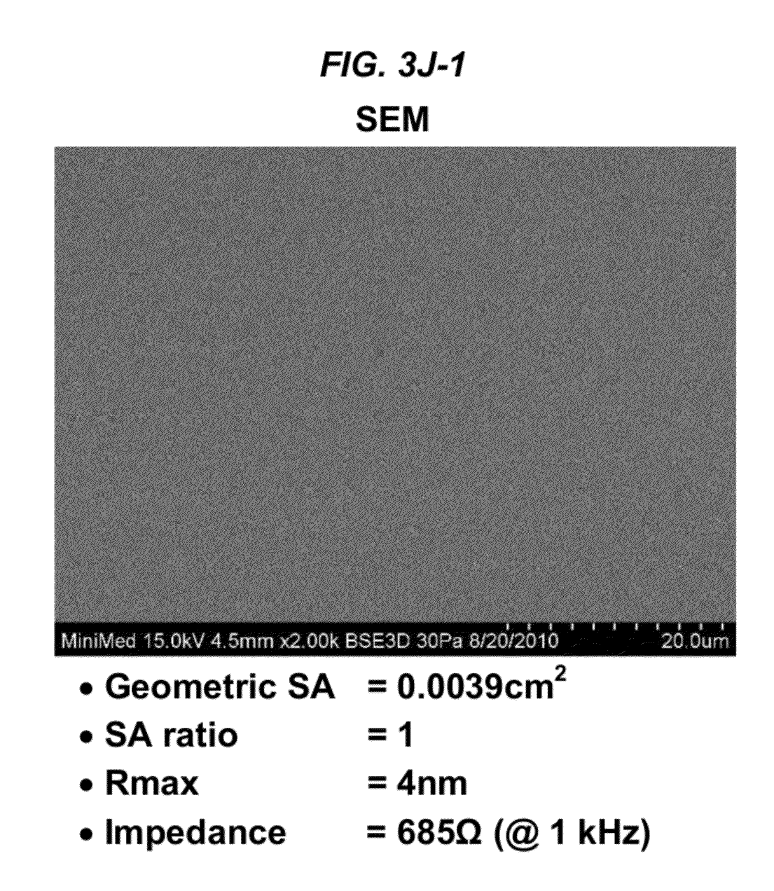

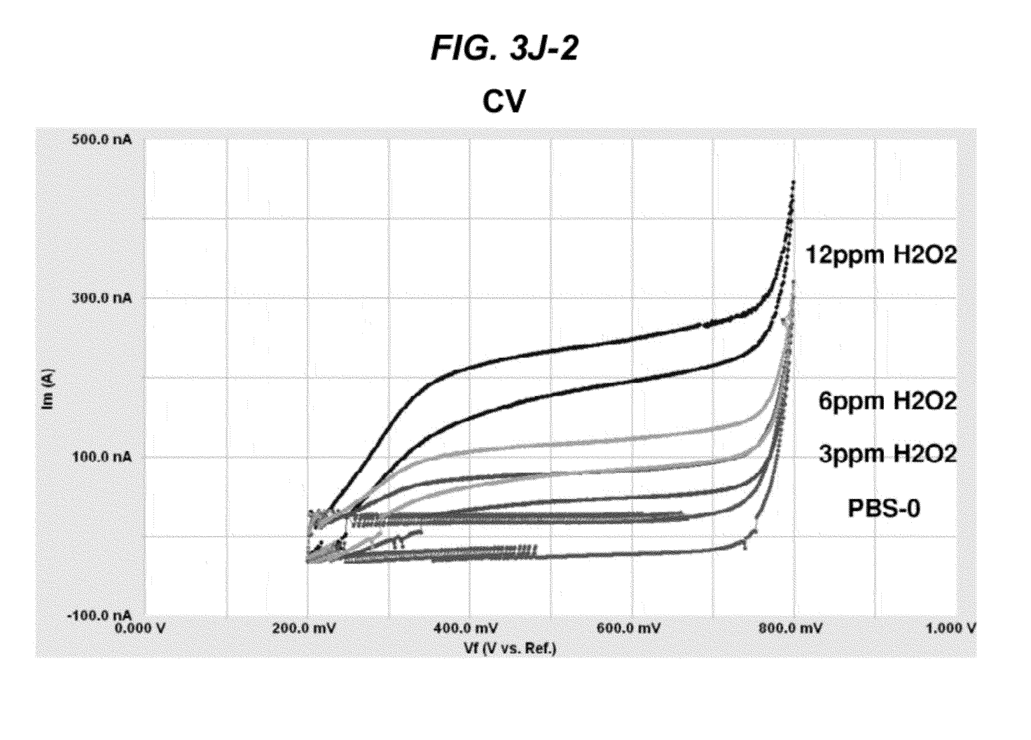

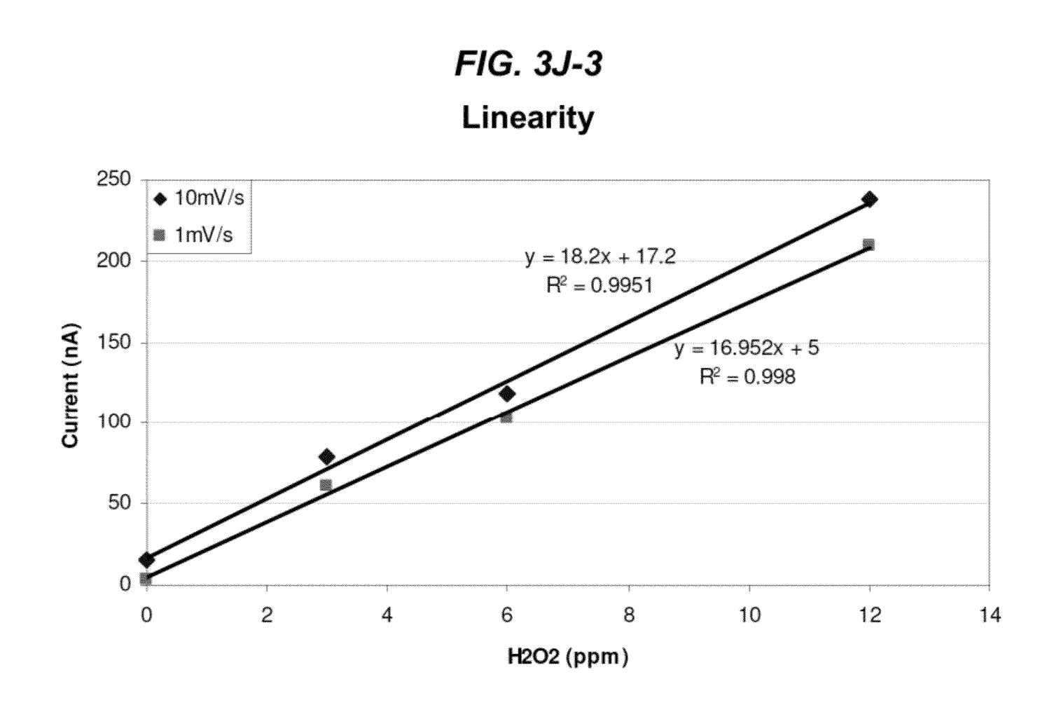

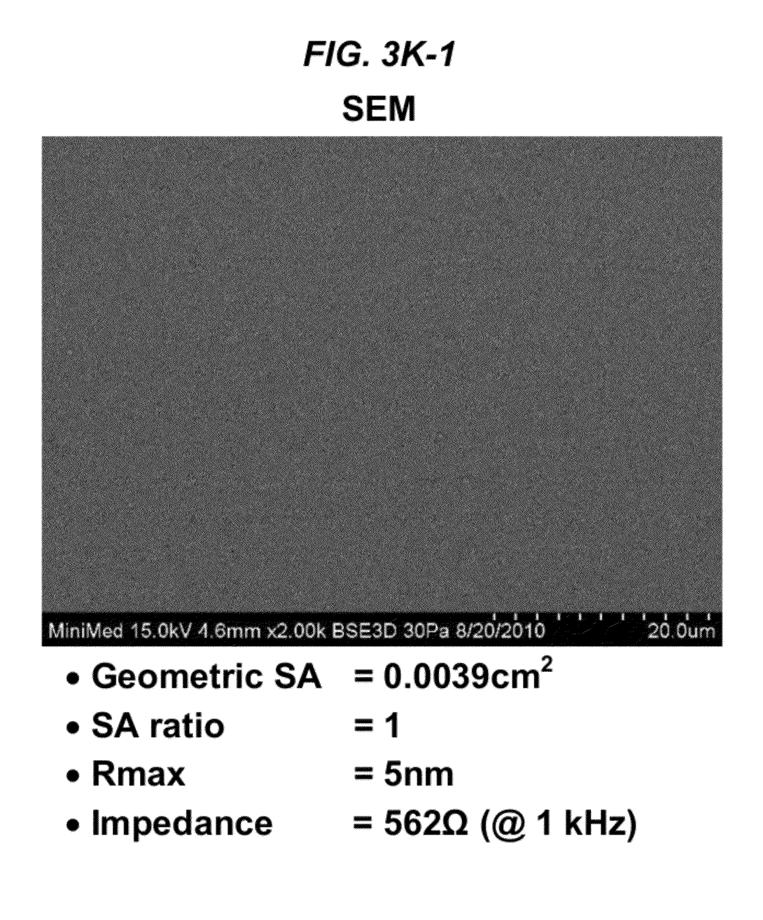

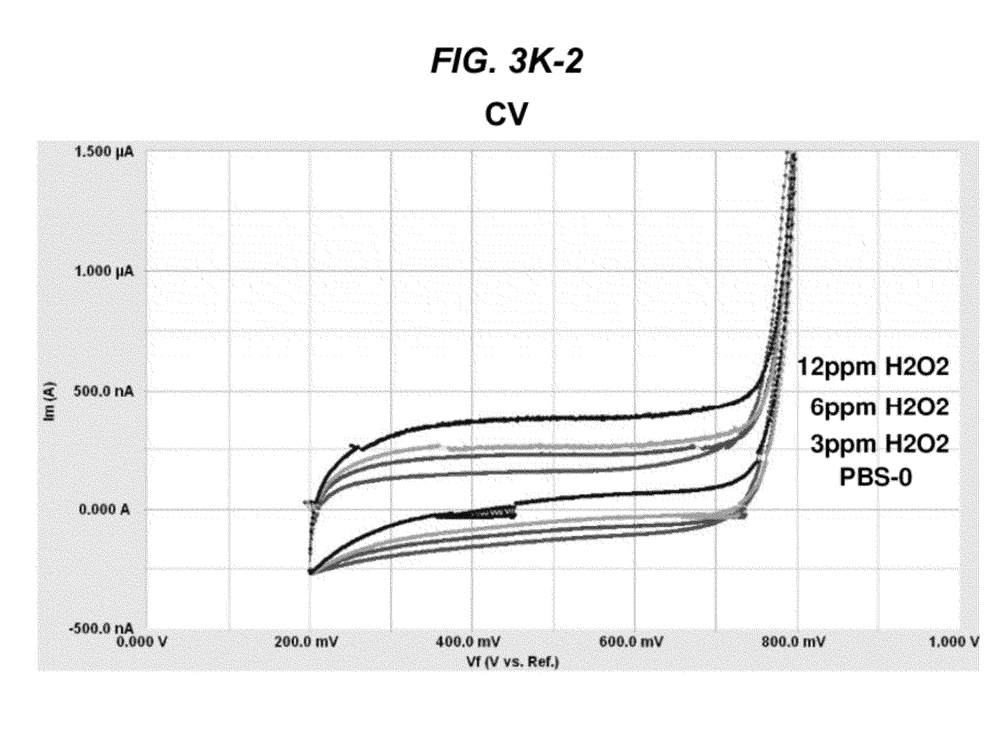

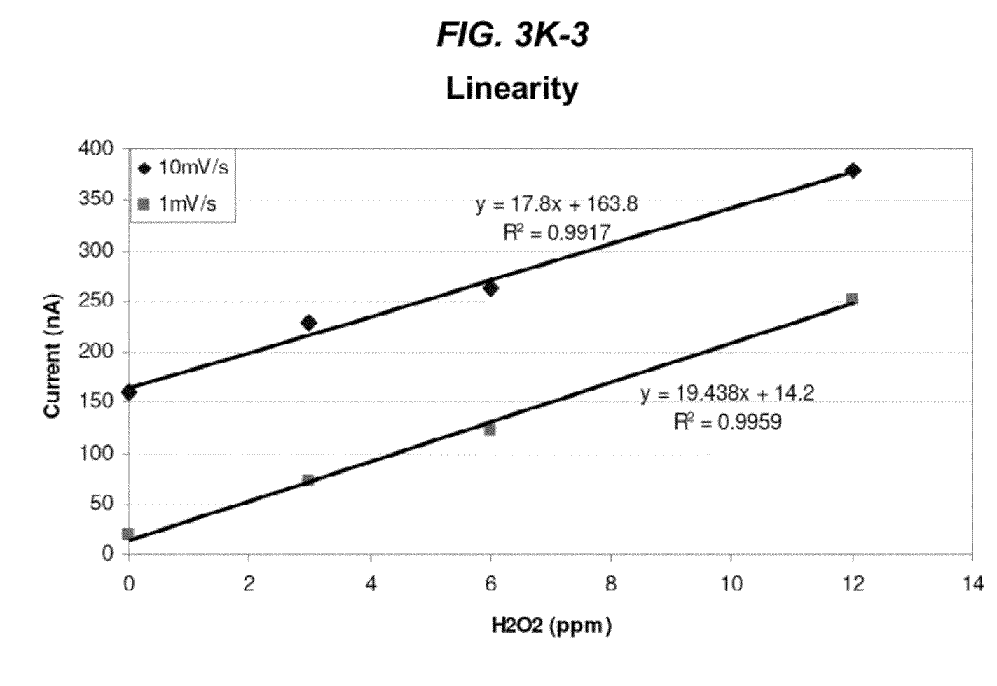

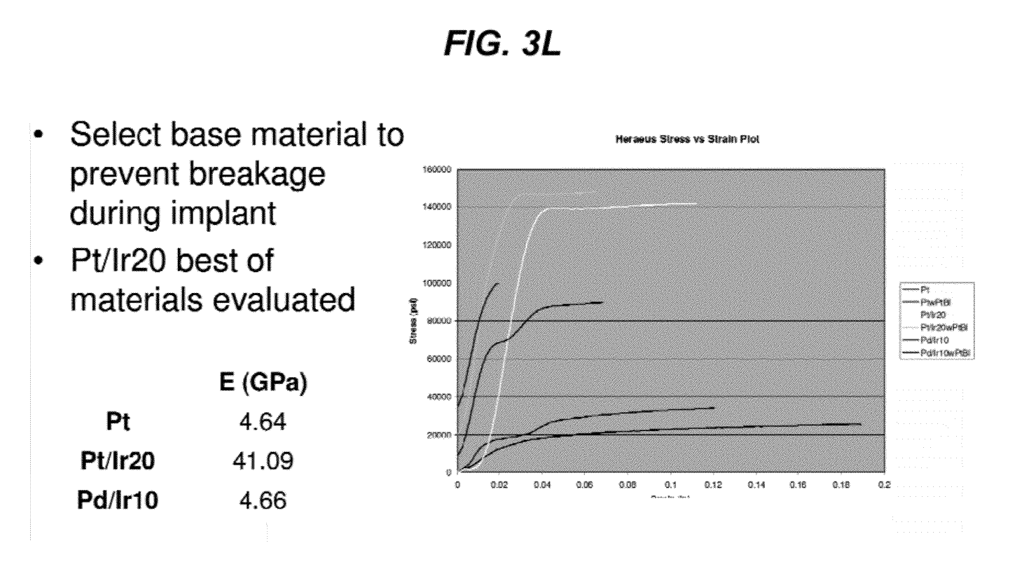

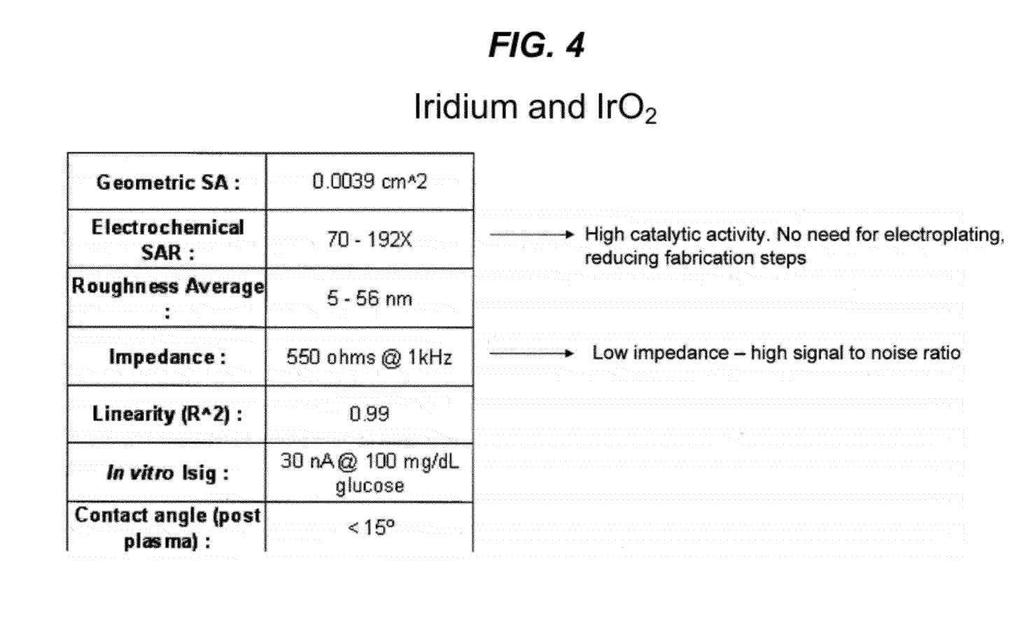

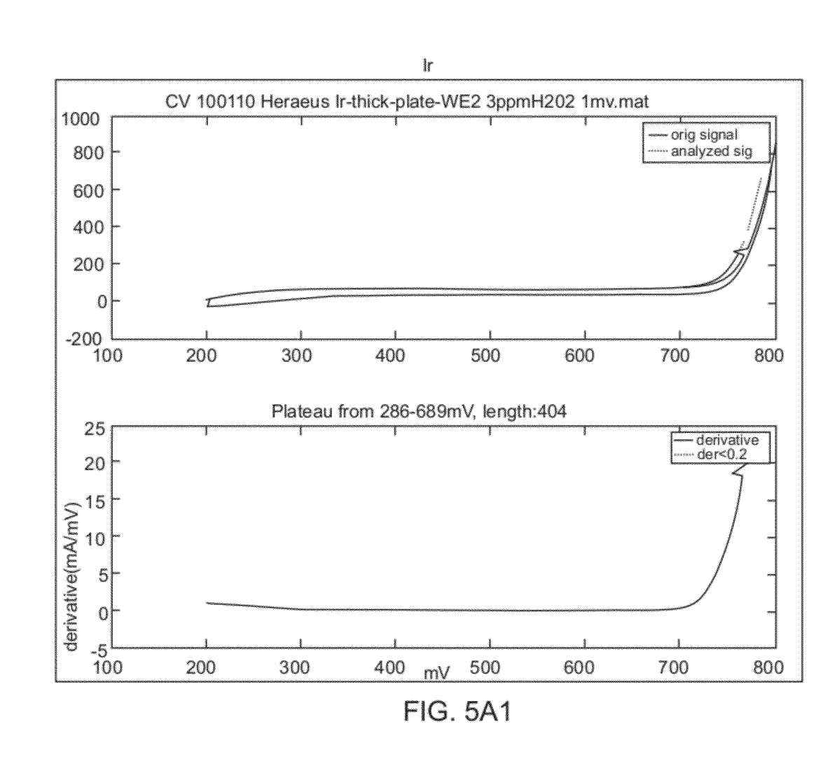

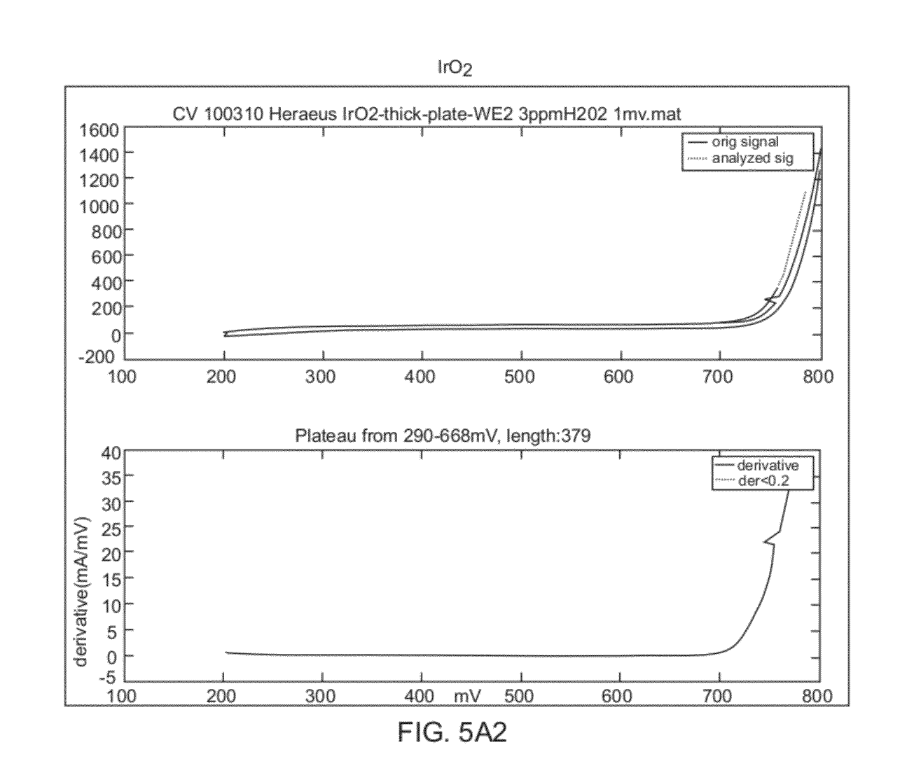

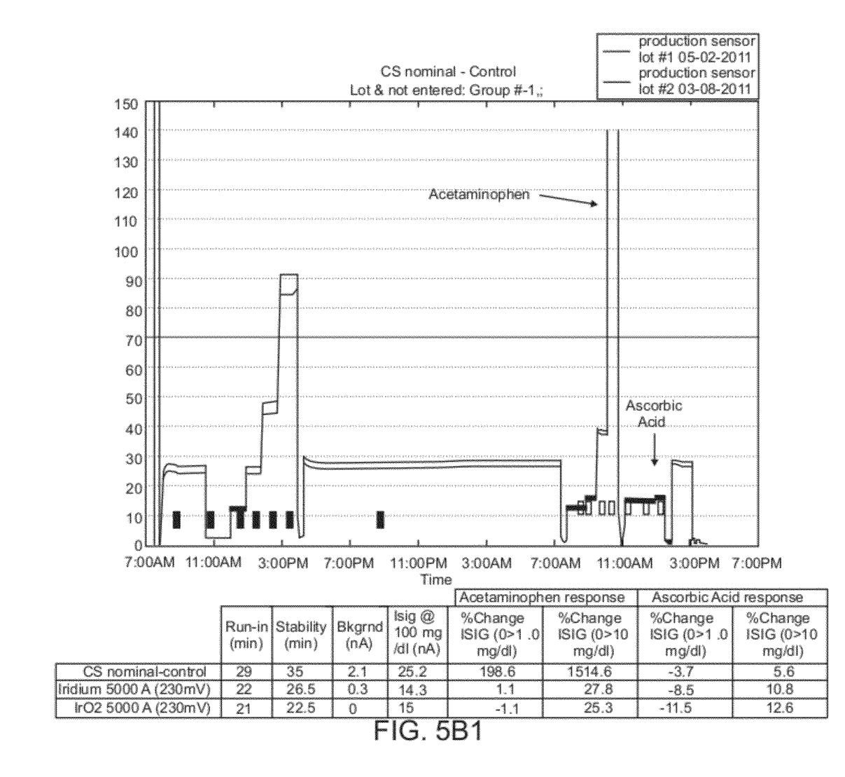

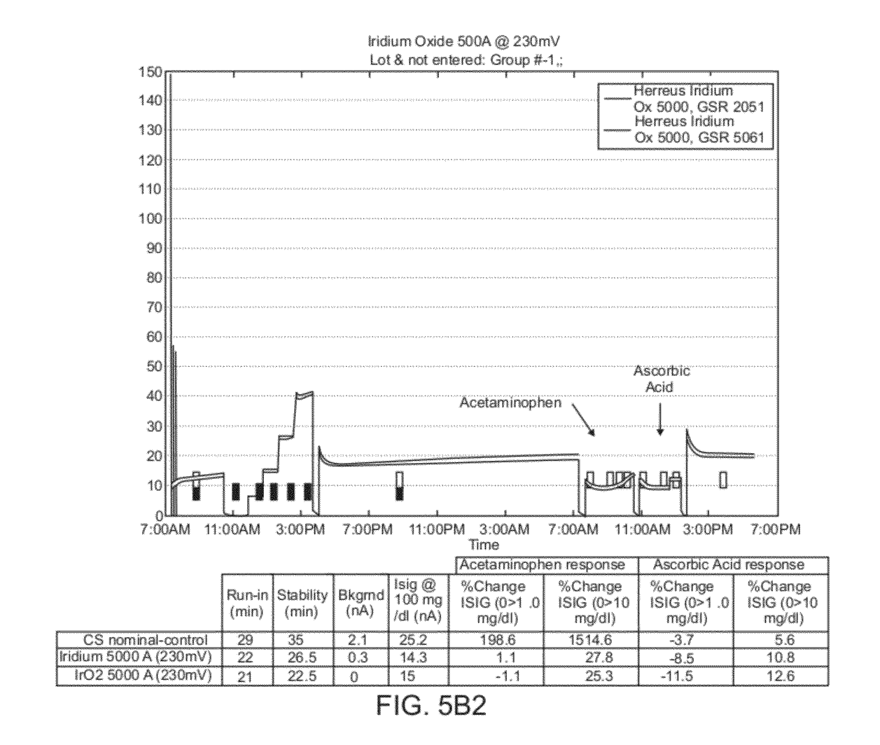

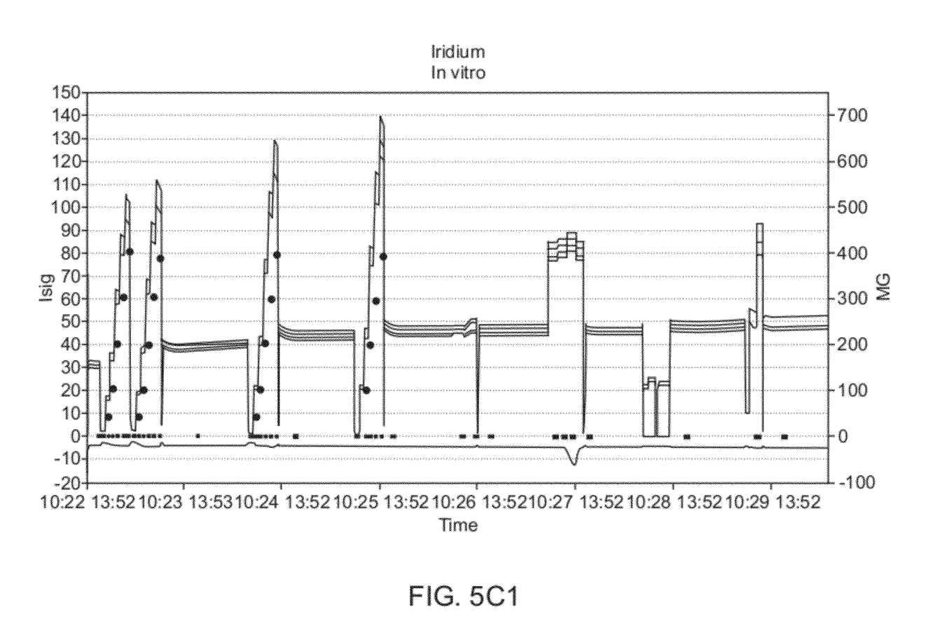

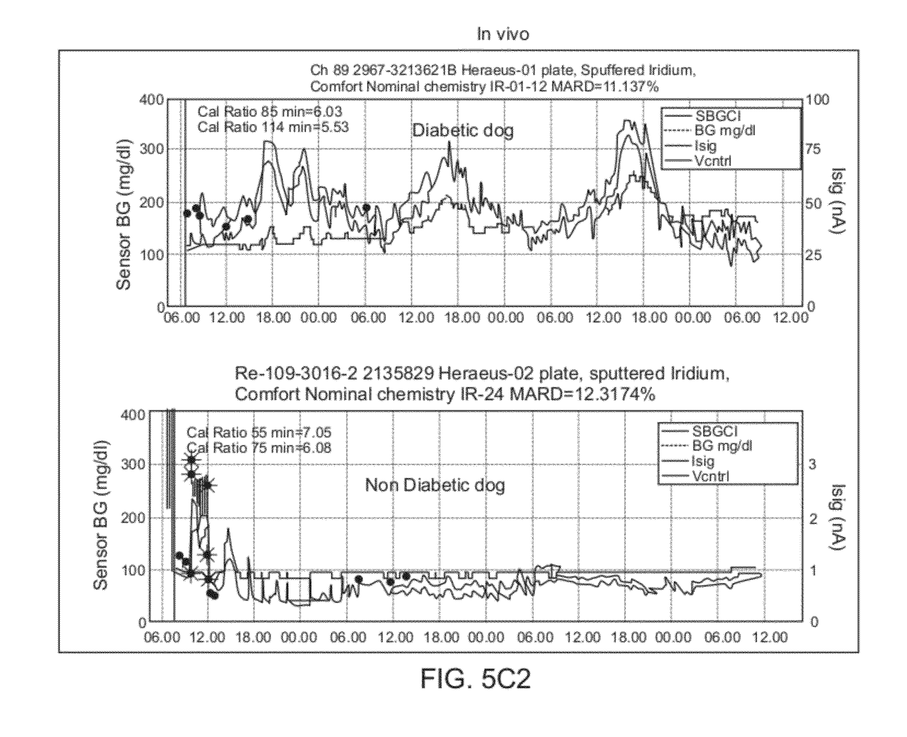

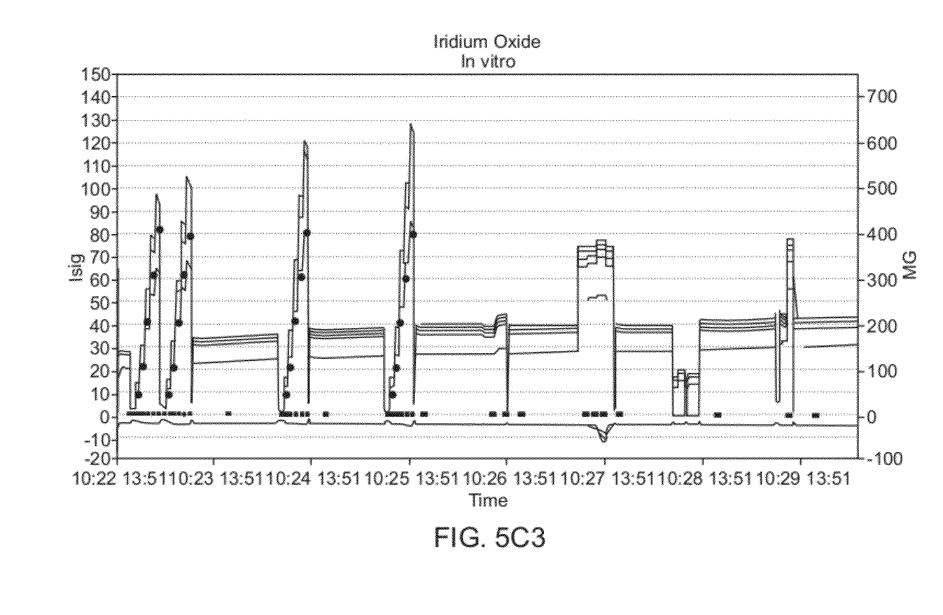

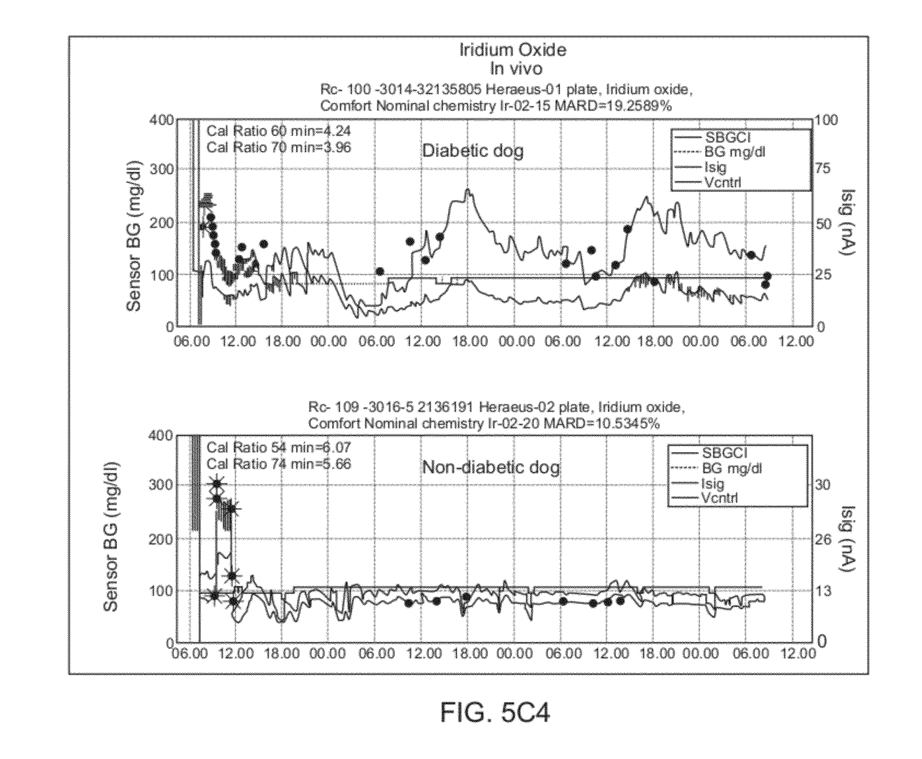

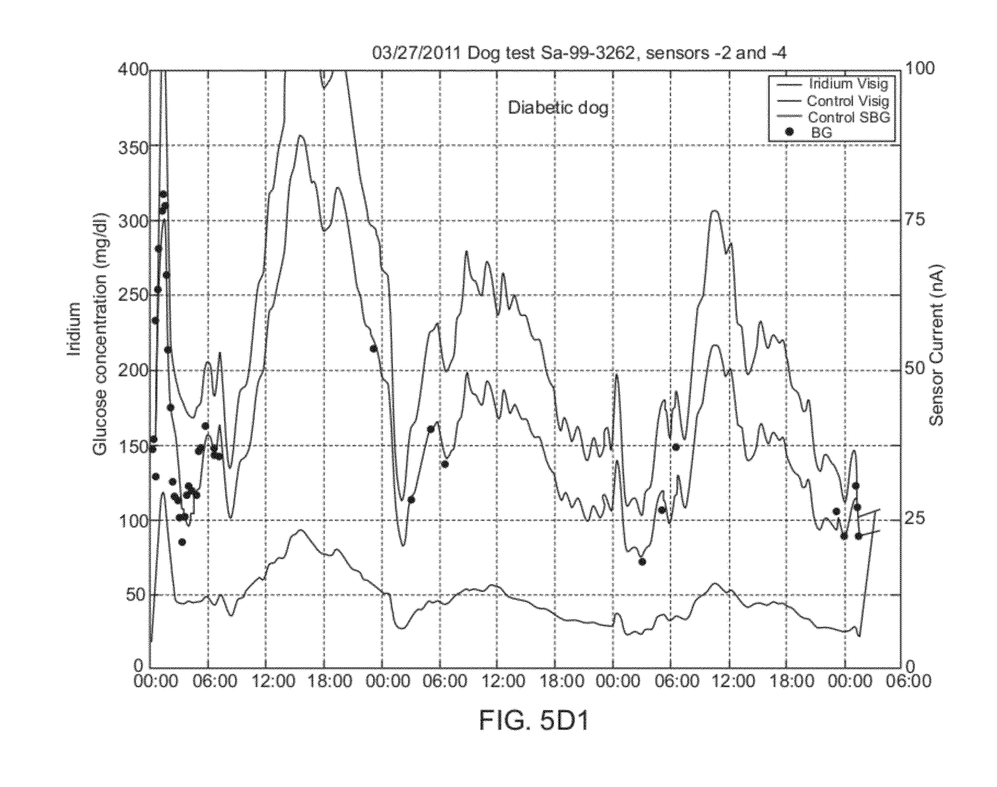

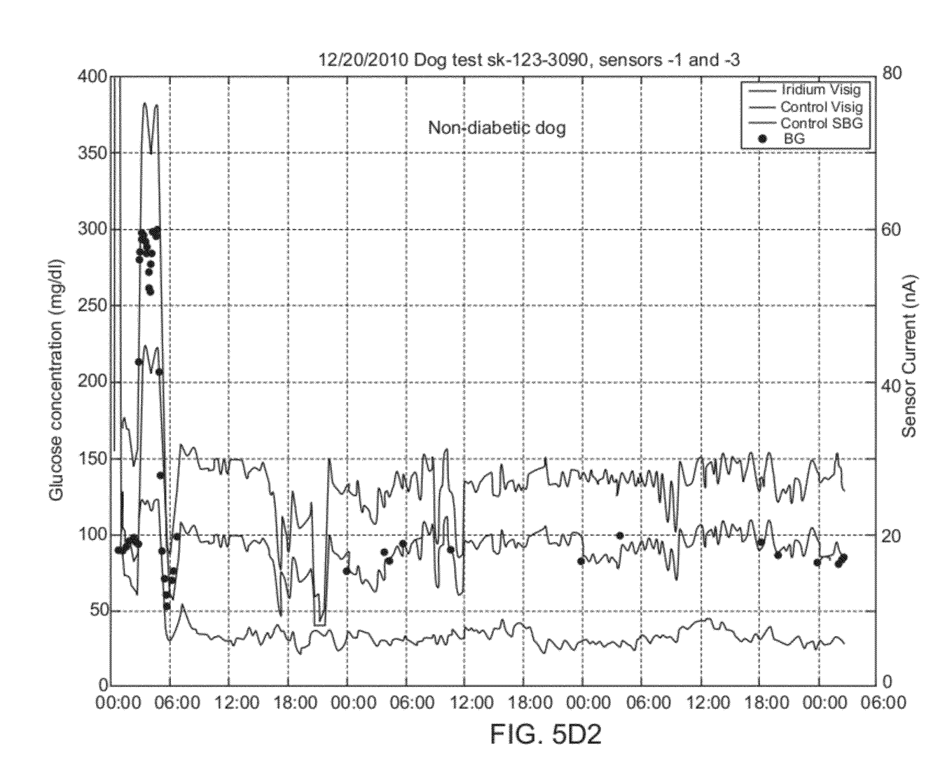

As discussed in detail below, embodiments of the invention utilize working electrodes comprising a first electrochemically reactive surface formed from an iridium composition such as iridium oxide (IrOx). The iridium metal in these working electrodes can be manipulated to exhibit a constellation of material properties that are useful in the glucose sensor embodiments disclosed herein. For example, the iridium working electrode embodiments exhibit a mechanical robustness appropriate for sensors placed in an in vivo environment (e.g. an environment in which sensors can twist and bend during use). In addition, iridium working electrode embodiments exhibit impedance profiles that (e.g. a lower impedance than Pt), for example, can be adapted to better identify signals that correlate with concentrations of analyte as compared to signals that result from background noise. Certain iridium working electrode embodiments also exhibit characteristics that can be useful with certain cyclic voltammetry methodologies, potentiodynamic electrochemical measurements useful in certain embodiments of amperometric glucose sensors. Moreover, when considering the relationship between the amount of current observed in the presence of differing hydrogen peroxide concentrations, certain iridium working electrode embodiments also exhibit a linearity that is useful when using glucose oxidases to generate hydrogen peroxide in the presence of glucose and oxygen over a wide range of glucose concentrations. Aspects of these characteristics of iridium working electrodes are shown in FIGS. 3-5.

The invention disclosed herein has a number of embodiments. One illustrative embodiment is an amperometric glucose sensor system comprising a processor that is operably coupled to a plurality of electrodes that are formed from distinct combinations of materials. Typically, these systems comprise a plurality of electrodes including a first working electrode comprising a first electrochemically reactive surface formed from an iridium (Ir) composition, wherein the first electrochemically reactive surface generates an electrochemical signal that is assessed by the processor in the presence of glucose; a second working electrode comprising a second electrochemically reactive surface formed from a platinum (Pt) composition, wherein the second electrochemically reactive surface generates an electrochemical signal that is assessed by the processor in the presence of glucose; a counter electrode; and a reference electrode. These systems can further include a computer-readable program code having instructions, which, when executed, cause the processor to: assess electrochemical signal data obtained from the first working electrode and the second working electrode; and then compute a glucose concentration based upon the electrochemical signal data obtained from the first working electrode and/or the second working electrode.

Embodiments of the glucose sensors systems disclosed herein comprise a first working electrode formed from iridium (for example iridium oxide) and adapted to sense blood glucose concentrations in a specific range, for example between 40-400 mg/dL; and/or between 40-100 mg/dL (e.g. to measure hypoglycemia); and/or between 70-400 mg/dL (e.g. to measure hyperglycemia). Embodiments of the glucose sensors disclosed herein further comprise a second working electrode formed from platinum (for example platinum black) and adapted to sense blood glucose concentrations in a specific range, for example between 40-400 mg/dL; and/or between 40-100 mg/dL (e.g. to measure hypoglycemia); and/or between 70-400 mg/dL (e.g. to measure hyperglycemia). In some embodiments of the invention, both the first and the second working electrode within the system are used to sense the total range of blood glucose concentrations observed in diabetic patients. In other embodiments of the invention, either the first or the second working electrode within the system is used to specifically focus on sensing blood glucose concentrations associated with either hypoglycemia or hyperglycemia. Certain embodiments of the invention include a computer-readable program code having instructions, which, when executed, cause the processor to assess a specified subset of electrochemical signal data obtained from the first working electrode and/or the second working electrode, wherein this subset of electrochemical signal data is that associated with a specific range of blood glucose concentrations, for example the range associated with either hypoglycemia or hyperglycemia

One illustrative embodiment is an amperometric glucose sensor system comprising a processor that is operably coupled to a plurality of electrodes that are formed from distinct combinations of materials. Typically, these systems comprise a plurality of electrodes including a first working electrode comprising a first electrochemically reactive surface formed from an iridium composition, wherein the first electrochemically reactive surface generates an electrochemical signal that is assessed by the processor in the presence of blood glucose concentrations in a specific range, for example between 40-100 mg/dL (or 70-400 mg/dL or 40-400 mg/dL); a second working electrode comprising a second electrochemically reactive surface formed from a platinum composition, wherein the second electrochemically reactive surface generates an electrochemical signal that is assessed by the processor in the presence of blood glucose concentrations in a specific range, for example between 70-400 mg/dL (or 40-100 mg/dL; or 40-400 mg/dL); a counter electrode; and a reference electrode. These systems can further include a computer-readable program code having instructions, which, when executed, cause the processor to: assess electrochemical signal data obtained from the first working electrode and the second working electrode; and then compute a blood glucose concentration based upon the electrochemical signal data obtained from the first working electrode and/or the second working electrode.

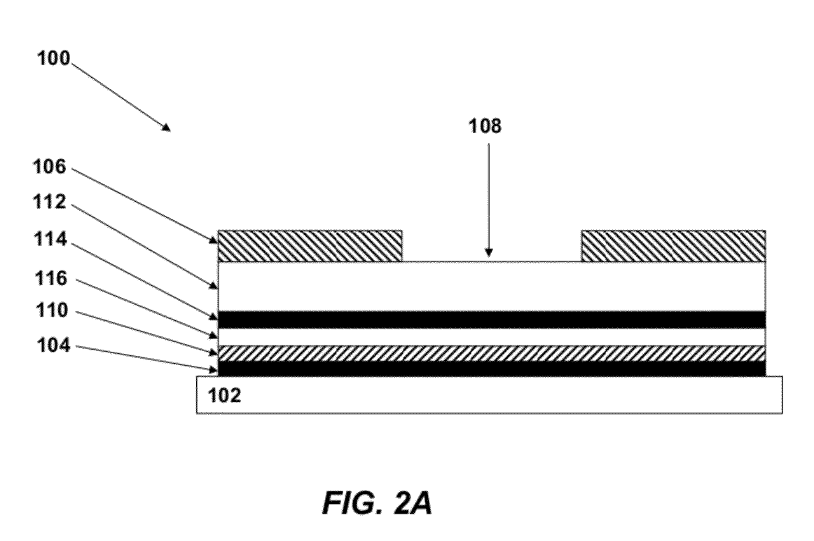

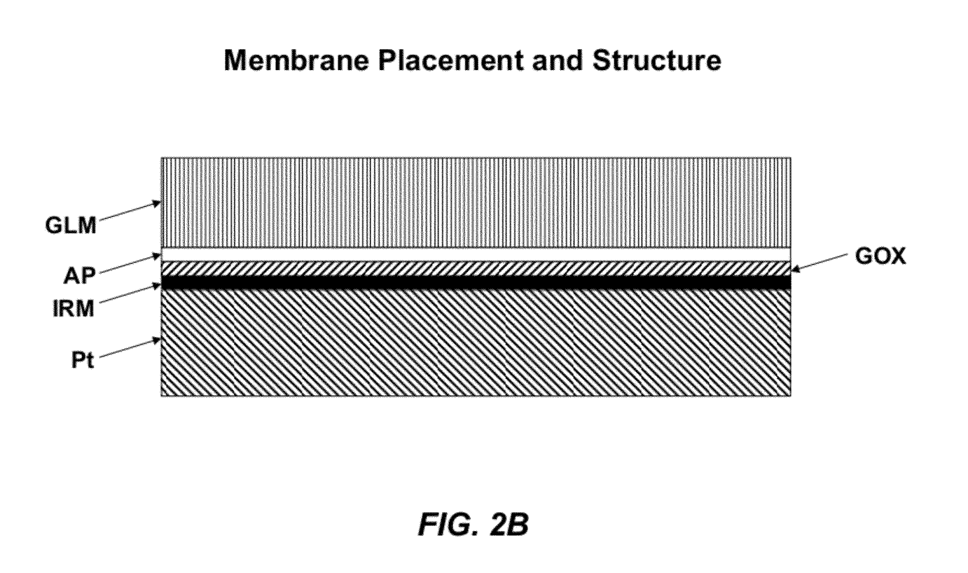

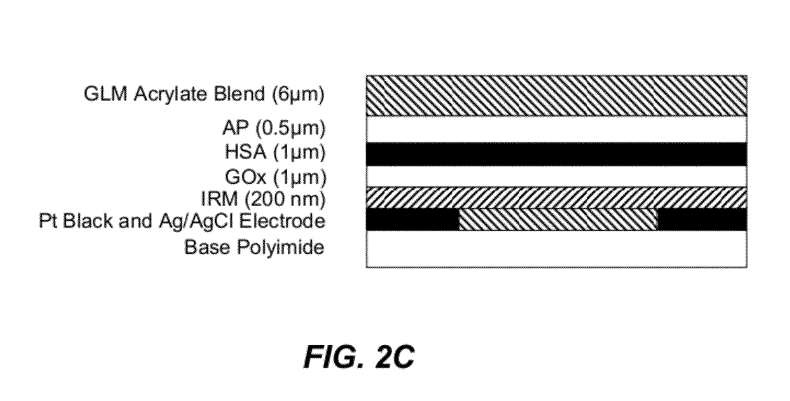

As noted above, embodiments of the glucose sensors disclosed herein comprise a first working electrode formed from iridium (for example iridium oxide) and adapted to sense blood glucose concentrations between 40-100 mg/dL (e.g. to measure hypoglycemia) and a second working electrode formed from platinum (for example platinum black) and adapted to sense blood glucose concentrations between 70-400 mg/dL (e.g. to measure hyperglycemia). Each of these working electrodes is typically coated with a plurality of layered compositions including, for example, a glucose oxidase composition; and/or an interference rejection composition; and/or a composition that modulates the diffusion of glucose therethrough. Illustrative non-limiting embodiments of such layered structures are shown for example in FIGS. 2A-2C.

Typically, the first and second working electrodes are coated with different layered materials. For example, in some embodiments of the invention, the first working electrode is not coated with an interference rejection layer and the second working electrode is coated with an interference rejection layer. In certain embodiments of the invention, the interference rejection layer comprises crosslinked primary amine polymers having an average molecular weight between 4 and 500 kilodaltons; or crosslinked Poly(2-hydroxyethyl methacrylate) polymers having an average molecular weight between 100 and 1000 kilodaltons. In certain embodiments of the invention, an interference rejection membrane (IRM) is characterized as having a specific response to an interfering compound, for example a sensor with one type of IRM has a 50% response (or has greater than or less than a 50% response) to 20 mg/dL acetaminophen. Interference rejection membranes that can be adapted for use with embodiments of the invention are further described in U.S. patent application Ser. No. 12/572,087, the contents of which are incorporated herein by reference.

In some embodiments of the invention, the first working electrode is coated with a first glucose oxidase layer and the second working electrode is coated with a second glucose oxidase layer, wherein the amount of glucose oxidase in the first glucose oxidase layer is greater than the amount of glucose oxidase in the second glucose oxidase layer. In some embodiments of the invention, this is accomplished by applying a glucose oxidase composition having a defined composition (e.g. a concentration of glucose oxidase between 35 KU/mL and 55 KU/mL) to both the first and the second working electrodes and applying a thicker layer of this glucose oxidase composition on the first working electrode so that the amount of glucose oxidase in the first glucose oxidase layer is greater than the amount of glucose oxidase in the second glucose oxidase layer (e.g. 110%, 120%, 130%, 140%, 150%, 160%, 170%, 180%, 190% or 200% greater). In other embodiments of the invention, this is accomplished by applying different glucose oxidase compositions having different concentrations on the first working electrode and the second working electrode, for example by applying a composition having a concentration of glucose oxidase between 35 KU/mL and 55 KU/mL (or between 30 KU/mL and 45 KU/mL etc.) to the first working electrode and then applying a composition having a concentration of glucose oxidase between 30 KU/mL and 45 KU/mL (or between 5 KU/mL and 15 KU/mL etc.) to the second working electrode so that the amount of glucose oxidase in the first glucose oxidase layer is greater than the amount of glucose oxidase in the second glucose oxidase layer. The application of different glucose oxidase compositions having different concentrations on the first working electrode and the second working electrode can, for example, help to optimize the stoichiometry of the chemical reactants that generate hydrogen peroxide in the presence of analyte at each or the different metal electrodes in the disclosed sensors. Glucose oxidase compositions that can be adapted for use with such embodiments of the invention are described in U.S. patent application Ser. No. 13/010,640, the contents of which are incorporated herein by reference.

In yet another illustration of working electrodes coated with different layered materials, the first working electrode can be coated with a first glucose modulating layer having a first rate of glucose diffusion and the second working electrode can be coated with a second glucose modulating layer having a second rate of glucose diffusion, wherein the first rate of glucose diffusion is less than the second rate of glucose diffusion. In some embodiments of the invention, this is accomplished by applying a glucose modulating layer having a defined concentration to both the first and the second working electrodes and applying a thicker layer of this glucose modulating composition on the first working electrode (e.g. 110%, 120%, 130%, 140%, 150%, 160%, 170%, 180%, 190% or 200% the thickness) so that the amount of glucose modulating material on the first working electrode is greater than the amount of glucose modulating material on the second glucose oxidase layer. In other embodiments of the invention, this is accomplished by applying glucose modulating compositions having different constituents (or concentrations of constituents) on the first working electrode and the second working electrode, for example by applying a layer of glucose modulating composition having an equivalent thickness to both the first and second working electrodes, with the layer on the first working electrode formed from constituents that produce a permeability that differs from the permeability of the layer on the second working electrode by 110%, 120%, 130%, 140%, 150%, 160%, 170%, 180%, 190% or 200%. Analyte modulating compositions that can be adapted for use with such embodiments of the invention are further described in U.S. patent application Ser. No. 12/643,790, the contents of which are incorporated herein by reference.

The application of different glucose oxidase compositions and/or glucose modulating compositions having different permeabilities on the first working electrode and the second working electrode as discussed above can, for example, be used to help optimize the stoichiometry of the chemical reactants that generate hydrogen peroxide in the presence of analyte at the iridium or the platinum electrodes in the disclosed sensors. Specifically, because the properties of a glucose oxidase composition or an analyte modulating composition can influence the rate at which a reaction proceeds, the material properties of a glucose oxidase layer and/or an analyte modulating layer used with electrochemical glucose sensor electrodes that utilize the chemical reaction between glucose and glucose oxidase to generate a measurable signal, can for example, be modulated in order to avoid oxygen deficit problems in each individual iridium or platinum electrode.

Embodiments of the invention include those where a surface feature or architecture of an electrode is designed to exhibit certain characteristics. For example, in certain embodiments of the invention, the first and/or second working electrode exhibits an electrochemically reactive surface area that is at least 25% greater than the geometrical surface area of the first working electrode. In yet another embodiment of the invention, the first and/or second working electrodes can be formed from a cylindrical wire having a diameter less than 0.0015 inches. In other embodiments of the invention, the first and/or second working electrodes are formed as planar rectangles. Optionally, the size of one of the working electrodes is at least 1.5, 2 or 2.5 folds larger than the size of the other working electrode.

Embodiments of the invention that comprise iridium electrodes rely on the specific material properties of iridium metal to influence the generation and/or sensing of signals at these electrodes. In this context, a variety of iridium electrodes are contemplated for use with embodiments of the amperometric glucose sensors disclosed herein. In some exemplary embodiments, the iridium is combined with other metals to form an alloy. Optionally for example, an iridium electrode comprises 10-90% iridium and 10-90% other metals (e.g. 80/20 or 20/80) such as palladium, platinum or ruthenium. In certain embodiments of the invention, the oxidation state of the metal is controlled in order to, for example, control the surface roughness of the electrode. In some exemplary embodiments, a layer of iridium or platinum coats a metallic substrate of the electrode. In certain embodiments of the invention, the electrode is coated on peripheral surfaces with an electrically conductive material layer consisting of iridium, iridium oxide, platinum and/or its alloys.

As noted above, the glucose sensor embodiments of the invention are typically coupled to a processor that facilitates the collection, storage and/or analysis of data obtained from the sensor electrodes. In certain embodiments of the invention, the processor evaluates data resulting from a plurality of different voltages applied to the system (e.g. a first voltage applied to the first working electrode and a second voltage applied to the second working electrode). In one illustrative embodiment of the invention, the processor evaluates data resulting from a plurality of different voltage pulses applied to the first working electrode that comprises iridium and the second working electrode that comprises platinum. In some embodiments of the invention, the data evaluated results from a voltage potential of between 0.2 and 0.6 volts applied to the first working electrode; and a voltage potential of between 0.5 and 0.7 volts applied to the second working electrode. In certain embodiments of the invention, the first and second electrodes generate: a current signal (Isig), wherein the current signal comprises a signal generated by the first and second electrodes in the presence of an analyte; and/or a voltage signal (Vcntr), wherein the voltage signal comprises a signal generated by the first and second working electrodes in response to voltage applied to the first and second electrodes. Illustrative methods that can be adapted for use with such embodiments of the invention are further described in U.S. patent application Ser. No. 12/345,354, the contents of which are incorporated herein by reference.

In some embodiments of the invention, the processor assesses electrochemical signal data obtained from the first working electrode and the second working electrode against one or more reliability parameters, ranks the electrochemical signal data obtained from the first working electrode and the second working electrode; and then computes blood glucose concentration based upon the ranking of electrochemical signal data obtained from the first working electrode and the second working electrode. In certain embodiments of the invention, the processor compares the electrochemical signal data from the first working electrode and the second working electrode in order to obtain information that, for example, provides an indication on how one or more electrochemical signals from the first working electrode or the second working electrode correlates with actual glucose blood concentrations in a diabetic patient. In typical embodiments of the invention, the comparison includes observing whether a signal obtained from the first working electrode and/or the second working electrode falls within a predetermined range of values. In other embodiments of the invention, the comparison includes observing a trend in sensor signal data from the first working electrode and/or the second working electrode. In yet other embodiments of the invention, the comparison includes observing an amount of nonspecific signal noise in the first working electrode and/or the second working electrode. Using embodiments of the invention disclosed herein, one can identify one or more signals observed by the sensor that is indicative of increasing blood glucose concentrations or decreasing blood glucose concentrations in the diabetic patient and/or is indicative of the presence of interfering compounds; and/or is indicative of background noise; and/or is indicative of sensor hydration; and/or is indicative of sensor signal drift; and/or is indicative of sensor loss of sensitivity to glucose. Illustrative processor functions that can be adapted for use with such embodiments of the invention are further described in U.S. patent application Ser. Nos. 12/914,969 and 13/165061, the contents of which are incorporated herein by reference.

Embodiments of the invention can comprise one or more probes adapted to be inserted in vivo and includes an electrode array comprising the first working electrode formed from an iridium composition, the second working electrode formed from a platinum composition, the counter electrode and the reference electrode. Some embodiments of the invention include multiple probes with discreet electrode arrays that are configured to be electronically independent of each other. Optionally the probes are coupled to a probe platform. In certain embodiments of the invention, a first and second probe are oriented on the probe platform so that the first and second electrode arrays are located at different depths when inserted into an in vivo environment. In other embodiments of the invention, a first probe and a second probe are coupled to the probe platform and the probe platform is made from a flexible material that allows the probes to twist and bend when implanted in vivo in a manner that inhibits in vivo movement of the probes. Other embodiments of the invention comprise an adhesive patch adapted to secure the probe(s) and/or the probe platform to the skin of a diabetic patient.

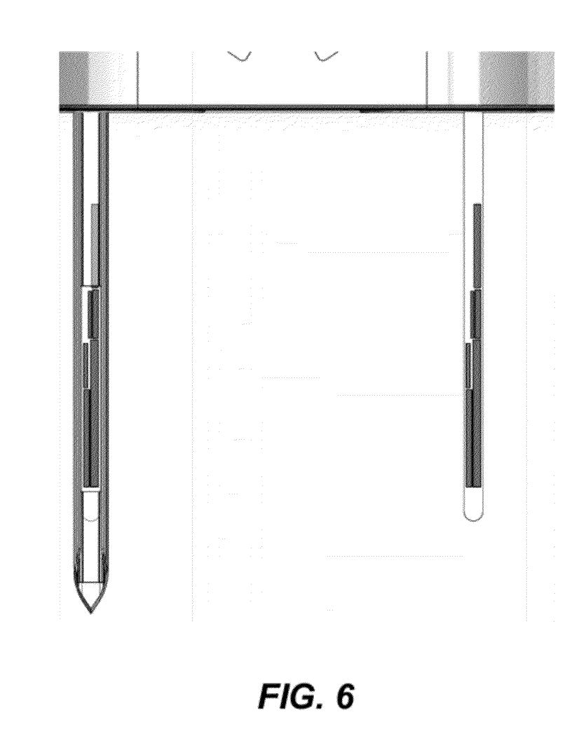

In some embodiments of the invention, the system further comprises a computer-readable program code having instructions, which when executed cause the processor to assess electrochemical signal data obtained from the first and second electrode arrays; and then compute a blood glucose concentration based upon the electrochemical signal data obtained from the first and second electrode arrays. In some embodiments, the electrochemical signal data obtained from the first working electrode and the second working electrode on an array is weighted according to one or more reliability parameters and the weighted electrochemical signal data is fused to compute an analyte concentration. In certain embodiments of the invention, electrochemical signal data obtained from the first and second electrode arrays is weighted according to one or more reliability parameters and the weighted electrochemical signal data is fused to compute an analyte concentration. Typically in these embodiments, the first array and second array are configured to be electronically independent of one another. Illustrative probe structures that can be adapted for use with such embodiments of the invention are shown in FIG. 6 and further described in U.S. patent application Ser. No. 13/165,061, the contents of which are incorporated herein by reference.

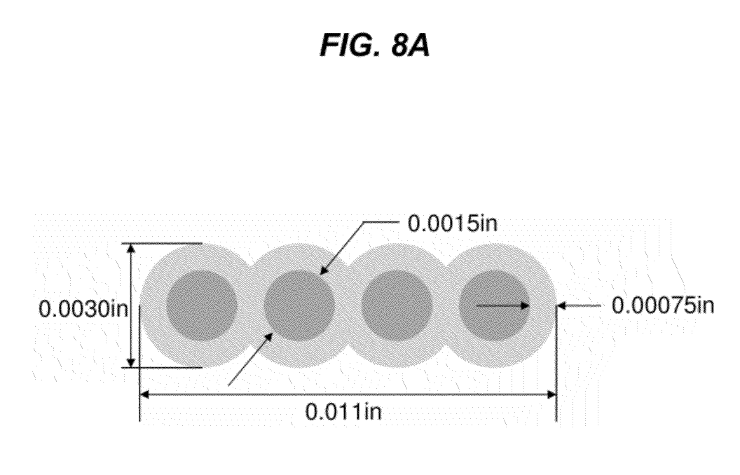

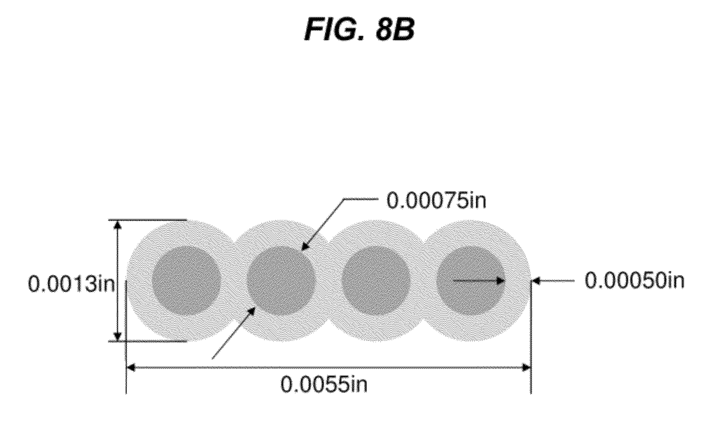

Embodiments of the invention can include an amperometric glucose sensor that is operatively coupled to a ribbon wire in order to, for example, allow for distributed sensing. Embodiments of such wires are shown in FIGS. 8A and 8B. In one embodiment, the sensor is operatively coupled to a multi-conductor electrical lead having a coiled configuration as disclosed in U.S. patent application Ser. No. 12/949,038, the contents of which are incorporated by reference herein. The compact architecture of the multi-conductor lead designs disclosed herein allows various elements in sensor systems to be electrically connected together in a space saving configuration, one that optimizes the use of such systems in a variety of contexts including situations where a patient is ambulatory and outside of a clinical environment, as well as conventional hospital environments. Illustrative embodiments of the invention include amperometric glucose sensor systems comprising a coiled conductor design where multiple conductive elements such as wires disposed within a ribbon cable are wrapped around a central core element in an arrangement that minimizes the space required for electrical leads used to operatively connect one element in the system to another.

Embodiments of the invention also include methods for computing blood glucose concentrations in a diabetic patient using the analyte sensor systems disclosed herein. In addition to methods for computing blood glucose concentrations in a diabetic patient, the analyte sensor systems disclosed herein can also be used in methods that are designed to assess sensor performance. In typical embodiments of the invention, the methods comprise observing electrochemical signal data generated by a sensor system comprising a processor; and a first working electrode comprising a first electrochemically reactive surface formed from an iridium composition, wherein the first electrochemically reactive surface generates an electrochemical signal that is assessed by the processor in the presence of blood glucose concentrations between 40-400 mg/dL and/or below 70 mg/dL (e.g. within the hypoglycemic range) and/or above 70 mg/dL (e.g. within the hyperglycemic range); a second working electrode comprising a second electrochemically reactive surface formed from a platinum composition, wherein the second electrochemically reactive surface generates an electrochemical signal that is assessed by the processor in the presence of blood glucose concentrations between 40-400 mg/dL and/or below 70 mg/dL (e.g. within the hypoglycemic range) and/or above 70 mg/dL (e.g. within the hyperglycemic range); a counter electrode; and a reference electrode. Typically the systems used in these methods further include a computer-readable program code having instructions, which when executed cause the processor to assess electrochemical signal data obtained from the first working electrode and the second working electrode; and compute a blood glucose concentration (and/or a parameter associated with sensor function) based upon the electrochemical signal data obtained from the first working electrode and the second working electrode.

Optionally in these methods, the first working electrode and second working electrode are configured to be electronically independent of one another; and the method further comprises: comparing the electrochemical signal data from the first working electrode and the second working electrode; and then computing blood glucose concentration using the comparison of the electrochemical signal data obtained from the first working electrode and the second working electrode. In certain embodiments of these methods, the comparison of electrochemical signal data from the first working electrode and the second working electrode includes observing whether a signal obtained from the first working electrode and the second working electrode falls within a predetermined range of values; or observing a trend in sensor signal data from the first working electrode and the second working electrode; or observing an amount of nonspecific signal noise in the first working electrode and/or the second working electrode.

Another embodiment of the invention is a composition of matter comprising an iridium composition having an electrochemically reactive surface; a glucose oxidase composition disposed upon the electrochemically reactive surface; and an analyte modulating layer disposed upon the glucose oxidase composition. In such embodiments of the invention, the analyte modulating layer typically comprises a linear polyurethane/polyurea polymer; a branched acrylate polymer; or a blended mixture of the linear polyurethane/polyurea polymer and the branched acrylate polymer, wherein the mixture is blended at a ratio of between 1:1 and 1:20 by weight percentage. In one illustrative embodiment of this composition, the linear polyurethane/polyurea polymer is formed from a mixture comprising: a diisocyanate; at least one hydrophilic diol or hydrophilic diamine; and a siloxane. In another illustrative embodiment of this composition, the branched acrylate polymer is formed from a mixture comprising a 2-(dimethylamino)ethyl methacrylate; a methyl methacrylate; a polydimethyl siloxane monomethacryloxypropyl; and a poly(ethylene oxide) methyl ether methacrylate.

Typically, the polyurethane/polyurea polymer used in such embodiments is formed from a mixture comprising: a diisocyanate compound (typically about 50 mol % of the reactants in the mixture); at least one hydrophilic diol or hydrophilic diamine compound (typically about 17 to 45 mol % of the reactants in the mixture); and a siloxane compound. Optionally the polyurethane/polyurea polymer comprises 45-55 mol % (e.g. 50 mol %) of a diisocyanate (e.g. 4,4′-diisocyanate), 10-20 (e.g. 12.5 mol %) mol % of a siloxane (e.g. polymethylhydrosiloxane, trimethylsilyl terminated), and 30-45 mol % (e.g. 37.5 mol %) of a hydrophilic diol or hydrophilic diamine compound (e.g. polypropylene glycol diamine having an average molecular weight of 600 Daltons, Jeffamine 600). Typically, the branched acrylate polymer used in such embodiments is formed from a mixture comprising: 5-45 weight % of a 2-(dimethylamino)ethyl methacrylate compound; 15-55 weight % of a methyl methacrylate compound; 15-55 weight % of a polydimethyl siloxane monomethacryloxypropyl compound; 5-35 weight % of a poly(ethylene oxide) methyl ether methacrylate compound; and 1-20 weight % 2-hydroxyethyl methacrylate.

Those of skill in the art understand that certain sensor and sensor system elements disclosed in one illustrative embodiment as disclosed herein can be substituted and/or combined with sensor and sensor system elements disclosed in another illustrative embodiment in order to form yet another embodiment of the invention. In this context, certain elements in some of the glucose sensor system embodiments of the invention can substituted with elements found in other glucose sensor system embodiments and/or combined with additional elements to facilitate their use in various contexts, for example a monitor adapted to display discreet signal information from the first working electrode and/or the second working electrode. One illustrative embodiment is an amperometric analyte sensor system comprising: a probe platform; a first probe coupled to the probe platform and adapted to be inserted in vivo (e.g. is made from a biocompatible materials, has a relatively smooth surface and an architecture designed to avoid unnecessary tissue damage upon insertion etc.), wherein the first probe comprises a first electrode array comprising a first and second working electrode having different material properties, a counter electrode and a reference electrode. Optionally, the first probe includes another electronically independent electrode array also comprising a first and second working electrode, a counter electrode and a reference electrode. This system can further include a second probe that is also coupled to the probe platform and adapted to be inserted in vivo, the second probe including at least one additional electronically independent electrode array comprising a first and second working electrode, a counter electrode and a reference electrode. In certain embodiments of the invention, the first or second probe contains 2, 3, 4, 5, 6 or more electronically independent electrode arrays, each comprising working electrodes having different material properties, a counter electrode and a reference electrode. Other embodiments of the invention can include 3, 4, 5 or more in vivo probes on which the independent electrode arrays are disposed. Illustrative architectural configurations that can be adapted for use with these sensor systems are shown in FIGS. 3-5 of U.S. application Ser. No. 13/165,061, the contents of which are incorporated herein by reference. Illustrative algorithms that can be adapted for use with these sensor systems are discussed in U.S. application Ser. No. 12/914,969 (see, e.g. paragraphs [0056]-[0125]), the contents of which are incorporated herein by reference.

As noted above, certain embodiments of the invention combine sensor structures/architectures disclosed herein with a processor to use combined or fused sensor signals to, for example, assess the reliability of a glucose sensor system. Such systems can, for example, monitor the sensor signals from multiple working electrodes having different material properties and then convert sensor signals to glucose value as well as provide information on the reliability of this signal information. In this way, the sensor systems disclosed herein can address a number of problems with sensor accuracy and reliability that are observed in this technology. In particular, as is known in the art, electrochemical analyte sensors can experience problems due to both the in vivo environment in which they are disposed as well as the functional degradation of the sensor components themselves. For example, the reliability of electrode array signals can be questionable in situations where an electrode array is inadvertently disposed in vivo at a site having suboptimal tissue properties (e.g. scar tissue) and/or is disposed at a suboptimal tissue depth (which can, for example, result in suboptimal hydration of the sensor).

Illustrative functionalities of the sensor systems disclosed herein include signal integrity checks. For example such systems can calculate internal reliability indexes (IRIs) and/or calculate and output a reliability index (RI) indicating sensor glucose (SG) reliability and/or calculate and output sensor status (OSS) for system control logic. Such systems can include calibration steps which, for example, convert each working electrode signal to sensor glucose (SG) based on input blood glucose (BG). Typically such systems include a sensor fusion function that examines (and optionally assigns a weight to) factors such as sensor glucose signals from each electronically independent working electrodes having different material properties and then “fuses” multiple signals to generate and output a single sensor glucose and/or reliability index (e.g. a reliability index for a single electrode array within the system and/or a comprehensive reliability index for the whole system). Illustrative SG outputs can include, for example, sensor glucose (e.g. in a concentration range of 40˜400 mg/dL; and/or 40˜70 mg/dL and/or 70˜400 mg/dL) that are calculated every minute. Illustrative reliability outputs measure how reliable the sensor signal and can, for example be formatted in a numerical range of 0˜1 and calculated every minute to provide four possible status indicators: pending (e.g. in sensor initialization and stabilization), good, bad, and failed. Artisans can use such system parameters to, for example detect sensor trends including a long-term, non-physiological trend, and/or a sensor failure as well as to characterize the noise of Isig in real-time.

In embodiments of the invention that evaluate signals derived from multiple working electrodes having different material properties against one or more reliability parameters, a reliability parameter can be calculated by a method comprising for example: determining whether a signal amplitude of one or more multiple working electrodes having different material properties falls within a predetermined range of amplitudes; and/or determining a trend in sensor signals from a plurality of signals sensed by multiple working electrodes having different material properties (e.g. so as to observe sensor signal drift in one or more arrays); and/or determining an amount of nonspecific signal noise sensed by one or more working electrodes having different material properties (e.g. in order to compare this signal to one or more predetermined internal noise parameters); and/or determining a mean value for signals obtained from the one or more working electrodes having different material properties (e.g. in order to compare this value to predetermined internal mean parameters); and/or determining a standard deviation for signals obtained from the one or more working electrodes having different material properties (e.g. in order to compare these values to predetermined internal standard deviation parameters). In typical embodiments of the invention, signal data recorded from each of the one or more working electrodes having different material properties is weighted according to one or more reliability parameters; and the weighted signal data is computationally fused to determine an analyte concentration. Optionally, signal data recorded from each of the one or more working electrodes having different material properties is assessed so as to provide an indication of: the status of the amperometric analyte sensor system comprising the multiple working electrodes having different material properties.

As noted above, in certain embodiments of the invention, the one or more working electrodes having different material properties are disposed within an electrode array. Typical electrode arrays comprise working electrodes, counter electrodes and reference electrodes. Optionally, the plurality of working, counter and reference electrodes are grouped together as a unit and positionally distributed on the conductive layer in a repeating pattern of units. Alternatively, the plurality of working, counter and reference electrodes are grouped together and positionally distributed on the conductive layer in a non-repeating pattern of units. In certain embodiments of the invention, an electrode array is coupled to an elongated base layer is made from a material that allows the sensor to twist and bend when implanted in vivo; and the electrodes are grouped in a configuration that facilitates an in vivo fluid to contact at least one of the working electrode as the sensor apparatus twists and bends when implanted in vivo.

In certain embodiments of the invention, the amperometric analyte sensor system comprises one or more elements designed to record, analyze and/or characterize signals received from the electrode arrays. For example, certain embodiments of the invention include a processor; a computer-readable program code having instructions, which when executed causes the processor to assess signal data obtained from each of the first, second, third and fourth electrode arrays by comparing this data to one or more reliability parameters; to rank signal data obtained from each of the first and second working electrodes and/or the first and second electrode arrays in accordance with this assessment; and to then compute an analyte concentration using ranked signal data from each of the first and second working electrodes and/or the first and second electrode arrays. Embodiments of the invention also typically include a number of additional components commonly used with analyte sensor systems, such as electrical conduits in operable contact with the various electrical elements of the system, monitors adapted to display signal information and, power sources adapted to be coupled to the electrode arrays etc.

Embodiments of the invention are designed to address certain general phenomena observed in sensor systems. For example, in some embodiments of the invention, the processor evaluates data provided by each of the individual working electrodes and/or electrode arrays so as to provide evidence of signal drift over time in the amperometric analyte sensor system. In some embodiments of the invention, the processor evaluates data so as to provide information on the initialization status of the amperometric analyte sensor system (e.g. data resulting from a plurality of amplitude pulses applied to the system). In such contexts, embodiments of the invention include using the analyte sensor system disclosed herein in methods designed to characterize the concentration of an analyte in an in vivo environment (e.g. glucose in a diabetic patient) and/or in methods designed to characterize the presence or levels of an interfering compound in an in vivo environment (e.g. acetaminophen, ascorbic acid etc.) and/or in methods of observing sensor signal drift (e.g. so as to observe sensor signal drift up or down over the in vivo lifetime of the sensor), and/or in methods of obtaining information on sensor start-up and initialization (e.g. to confirm that the sensor is ready to begin providing and/or characterizing information relating to blood glucose concentrations in a diabetic patient).

In addition to the sensor structures discussed above, embodiments of the invention relate to using these specific sensor structures in methods, systems, apparatuses, and/or articles, etc. for glucose sensor signal reliability analysis. In this context, glucose monitoring systems, including ones that are designed to adjust the glucose levels of a patient and/or to operate continually (e.g., repeatedly, at regular intervals, at least substantially continuously, etc.), may comprise a glucose sensor signal that may be assessed for reliability. More specifically, but by way of example only, reliability assessment(s) on glucose sensor signals may include glucose sensor signal stability assessment(s) to detect an apparent change in responsiveness of a signal.

Embodiments of the invention further include using the disclosed sensor architectures and/or sensor algorithms in methods for sensing analytes in vivo (e.g. glucose concentrations in a diabetic patient). Typically, the method comprises observing signal data generated by a plurality of working electrodes having different material properties in the presence of analyte, and then using this observed signal data to compute an analyte concentration. Such methods can include, for example, comparing signal data from each of the working electrodes having different material properties and observing whether a signal obtained from each of the working electrodes falls within a predetermined range of values; and/or observing a trend in sensor signal data from each of the working electrodes and/or observing an amount of nonspecific signal noise in each of the working electrodes. In some embodiments of these methods, a comparison of the signal data obtained from the different working electrodes is used to identify a signal from an array that is indicative of increasing blood glucose concentrations or decreasing blood glucose concentrations in the diabetic patient; and/or a signal that is indicative of insufficient sensor hydration; and/or a signal that is indicative of sensor signal drift; and/or a signal that is indicative of sensor loss of sensitivity to analyte (e.g. due to sensor component degradation). In certain embodiments the methods comprise assigning a weighted value to signal data obtained from each of the working electrodes; and using the weighted signal values to compute an analyte concentration by fusing the various weighted signal values. Other embodiments of the invention include using the processor to: assess signal data from each of the working electrodes; and generate reliability index that indicates the reliability of a signal obtained from one or more of the working electrodes.

The analyte sensor systems disclosed herein can also be used in methods that are designed to assess sensor lifetime. For example, in embodiments of the analyte sensors systems that comprise an iridium electrode that oxidizes during its use, the levels of this oxidation can be used to monitor the duration of sensor use and, for example, prevent a diabetic patient from using the sensors beyond their recommended lifetime. In one illustrative embodiments of this, the method comprises using an Electrochemical Impedance Spectroscopy (EIS) procedure to monitor oxidation of the iridium electrode and then correlate this phenomena with sensor life. Optionally, the method comprises performing an EIS procedure in order to compare the impedance value against a defined such as the impedance value of unused sensors and/or the impedance value associated with sensors that have reached the end of their lifetime. In one illustrative embodiment, the method comprises performing an EIS procedure between at least two electrodes of the sensor, calculating an impedance value between the electrodes, and compares the impedance value against a threshold (e.g. to determine if the sensor has aged beyond the specified sensor life). Illustrative EIS procedures that can be adapted for use with such embodiments of the invention are described in U.S. Pat. No. 7,985,330, the contents of which are incorporated herein by reference.

In some embodiments of the invention, a sensing methodology may include: generating an alert signal responsive to a comparison of the at least one metric assessing an underlying trend with at least one predetermined threshold. In at least one example implementation, the assessing may include comparing the at least one metric assessing an underlying trend with at least a first predetermined threshold and a second predetermined threshold. In at least one other example implementation, the assessing may further include: assessing that the reliability of the at least one sensor signal is in a first state responsive to a comparison of the at least one metric assessing an underlying trend with the first predetermined threshold; assessing that the reliability of the at least one sensor signal is in a second state responsive to a comparison of the at least one metric assessing an underlying trend with the first predetermined threshold and the second predetermined threshold; and assessing that the reliability of the at least one sensor signal is in a third state responsive to a comparison of the at least one metric assessing an underlying trend with the second predetermined threshold. In at least one other example implementation, the assessing may further include: ascertaining at least one value indicating a severity of divergence by the at least one sensor signal from the blood glucose level of the patient over time based at least partly on the at least one metric assessing an underlying trend, the first predetermined threshold, and the second predetermined threshold. Illustrative algorithms that can be adapted for use with these sensor systems are discussed in U.S. application Ser. No. 12/914,969 (see, e.g. paragraphs [0056]-[0125]), the contents of which are incorporated herein by reference.

In other embodiments of the invention, a sensing methodology may include: acquiring the at least one sensor signal from one or more subcutaneous glucose sensor electrodes, wherein the at least one metric assessing an underlying trend may reflect an apparent reliability of the at least one signal that is acquired from the one or more subcutaneous glucose sensor working electrodes. In at least one example implementation, the method may further include: altering an insulin infusion treatment for the patient responsive at least partly to the assessed reliability of the at least one sensor signal.

In at least one example implementation, the determining may include: producing the at least one metric assessing an underlying trend using a slope of a linear regression that is derived at least partly from the series of samples of the at least one sensor signal. In at least one other example implementation, the method may include: transforming the series of samples of the at least one sensor signal to derive a monotonic curve, wherein the producing may include calculating the slope of the linear regression, with the linear regression being derived at least partly from the monotonic curve.

In at least one example implementation, the determining may include: decomposing the at least one sensor signal as represented by the series of samples using at least one empirical mode decomposition and one or more spline functions to remove relatively higher frequency components from the at least one sensor signal. In at least one example implementation, the determining may include: decomposing the at least one sensor signal as represented by the series of samples using at least one discrete wavelet transform; and reconstructing a smoothed signal from one or more approximation coefficients resulting from the at least one discrete wavelet transform. In at least one example implementation, the determining may include: iteratively updating a trend estimation at multiple samples of the series of samples of the at least one sensor signal based at least partly on a trend estimation at a previous sample and a growth term.

In one or more example embodiments, an apparatus may include: a controller to obtain a series of samples of at least one sensor signal that is responsive to a blood glucose level of a patient, and the controller may include one or more processors to: determine, based at least partly on the series of samples, at least one metric assessing an underlying trend of a change in responsiveness of the at least one sensor signal to the blood glucose level of the patient over time; and assess a reliability of the at least one sensor signal to respond to the blood glucose level of the patient based at least partly on the at least one metric assessing an underlying trend. In at least one example implementation, the one or more processors of the controller may further be to: generate an alert signal responsive to a comparison of the at least one metric assessing an underlying trend with at least one predetermined threshold. Optionally, a first metric is associated with the first working electrode comprising iridium (e.g. a “low signal metric”) and a second metric is associated with the second working electrode comprising platinum (e.g. a “high signal metric”).

In at least one example implementation, the controller may be capable of assessing by comparing the at least one metric assessing an underlying trend with at least a first predetermined threshold and a second predetermined threshold. In at least one other example implementation, the controller may be further capable of assessing by: assessing that the reliability of the at least one sensor signal is in a first state responsive to a comparison of the at least one metric assessing an underlying trend with the first predetermined threshold; assessing that the reliability of the at least one sensor signal is in a second state responsive to a comparison of the at least one metric assessing an underlying trend with the first predetermined threshold and the second predetermined threshold; and assessing that the reliability of the at least one sensor signal is in a third state responsive to a comparison of the at least one metric assessing an underlying trend with the second predetermined threshold. In at least one other example implementation, the controller may be further capable of assessing by: ascertaining at least one value indicating a severity of divergence by the at least one sensor signal from the blood glucose level of the patient over time based at least partly on the at least one metric assessing an underlying trend, the first predetermined threshold, and the second predetermined threshold.

In one or more example embodiments, an article may include at least one storage medium having stored thereon instructions executable by one or more processors to: obtain a series of samples of at least one sensor signal that is responsive to a blood glucose level of a patient; determine, based at least partly on the series of samples, at least one metric assessing an underlying trend of a change in responsiveness of the at least one sensor signal to the blood glucose level of the patient over time; and assess a reliability of the at least one sensor signal to respond to the blood glucose level of the patient based at least partly on the at least one metric assessing an underlying trend.

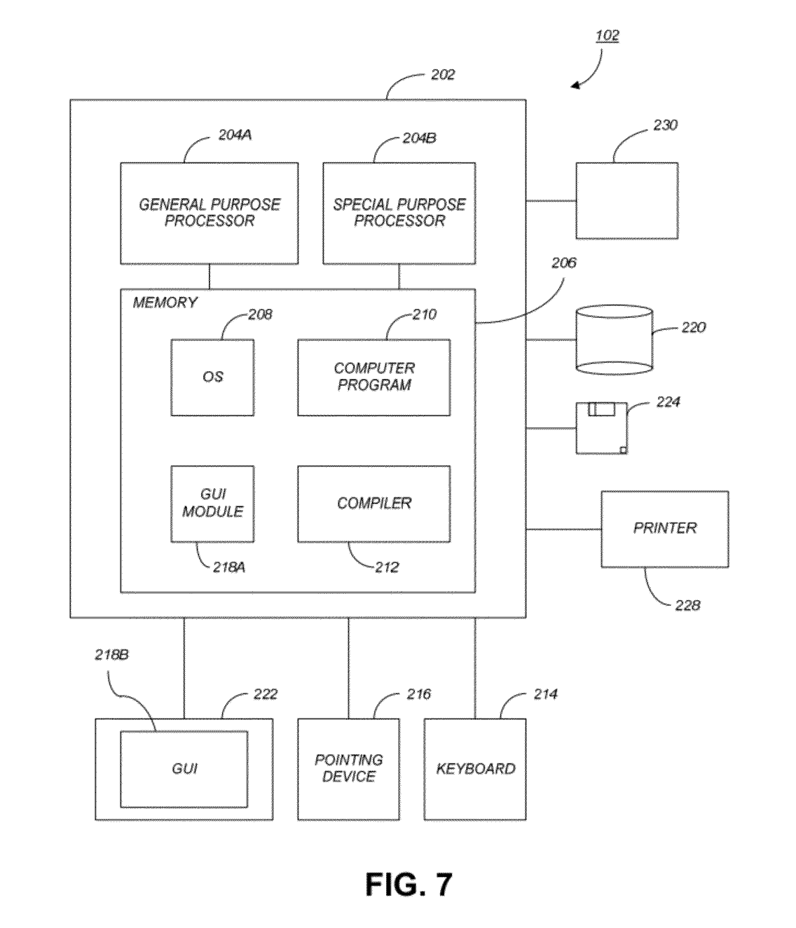

Embodiments of the invention disclosed herein can be performed for example, using one of the many computer systems known in the art. FIG. 7 illustrates an exemplary generalized computer system 202 that can be used to implement elements the present invention, including the user computer 102, servers 112, 122, and 142 and the databases 114, 124, and 144. The computer 202 typically comprises a general purpose hardware processor 204A and/or a special purpose hardware processor 204B (hereinafter alternatively collectively referred to as processor 204) and a memory 206, such as random access memory (RAM). The computer 202 may be coupled to other devices, including input/output (I/O) devices such as a keyboard 214, a mouse device 216 and a printer 228.

In one embodiment, the computer 202 operates by the general purpose processor 204A performing instructions defined by the computer program 210 under control of an operating system 208. The computer program 210 and/or the operating system 208 may be stored in the memory 206 and may interface with the user 132 and/or other devices to accept input and commands and, based on such input and commands and the instructions defined by the computer program 210 and operating system 208 to provide output and results. Output/results may be presented on the display 222 or provided to another device for presentation or further processing or action. In one embodiment, the display 222 comprises a liquid crystal display (LCD) having a plurality of separately addressable liquid crystals. Each liquid crystal of the display 222 changes to an opaque or translucent state to form a part of the image on the display in response to the data or information generated by the processor 204 from the application of the instructions of the computer program 210 and/or operating system 208 to the input and commands. The image may be provided through a graphical user interface (GUI) module 218A. Although the GUI module 218A is depicted as a separate module, the instructions performing the GUI functions can be resident or distributed in the operating system 208, the computer program 210, or implemented with special purpose memory and processors.

Some or all of the operations performed by the computer 202 according to the computer program 110 instructions may be implemented in a special purpose processor 204B. In this embodiment, the some or all of the computer program 210 instructions may be implemented via firmware instructions stored in a read only memory (ROM), a programmable read only memory (PROM) or flash memory in within the special purpose processor 204B or in memory 206. The special purpose processor 204B may also be hardwired through circuit design to perform some or all of the operations to implement the present invention. Further, the special purpose processor 204B may be a hybrid processor, which includes dedicated circuitry for performing a subset of functions, and other circuits for performing more general functions such as responding to computer program instructions. In one embodiment, the special purpose processor is an application specific integrated circuit (ASIC).

The computer 202 may also implement a compiler 212 which allows an application program 210 written in a programming language such as COBOL, C++, FORTRAN, or other language to be translated into processor 204 readable code. After completion, the application or computer program 210 accesses and manipulates data accepted from I/O devices and stored in the memory 206 of the computer 202 using the relationships and logic that was generated using the compiler 212. The computer 202 also optionally comprises an external communication device such as a modem, satellite link, Ethernet card, or other device for accepting input from and providing output to other computers.

In one embodiment, instructions implementing the operating system 208, the computer program 210, and the compiler 212 are tangibly embodied in a computer-readable medium, e.g., data storage device 220, which could include one or more fixed or removable data storage devices, such as a zip drive, floppy disc drive 224, hard drive, CD-ROM drive, tape drive, etc. Further, the operating system 208 and the computer program 210 are comprised of computer program instructions which, when accessed, read and executed by the computer 202, causes the computer 202 to perform the steps necessary to implement and/or use the present invention or to load the program of instructions into a memory, thus creating a special purpose data structure causing the computer to operate as a specially programmed computer executing the method steps described herein. Computer program 210 and/or operating instructions may also be tangibly embodied in memory 206 and/or data communications devices 230, thereby making a computer program product or article of manufacture according to the invention. As such, the terms “article of manufacture,” “program storage device” and “computer program product” as used herein are intended to encompass a computer program accessible from any computer readable device or media.

Of course, those skilled in the art will recognize that any combination of the above components, or any number of different components, peripherals, and other devices, may be used with the computer 202. Although the term “user computer” is referred to herein, it is understood that a user computer 102 may include portable devices such as glucose sensors, and other analyte sensing apparatuses, medication infusion pumps, cellphones, notebook computers, pocket computers, or any other device with suitable processing, communication, and input/output capability.

### Typical Sensor Layers Found in Embodiments of the Invention

As noted above, one or more of the electrodes of the invention (e.g. the first and/or second working electrode) is coated with one or more layers of various compositions that, like the specific metal used to form the electrode, can further modulate the functional properties of the working electrodes. Those of skill in this art will understand that not all material layers disclosed herein are used in every embodiment of the invention, and for example, that some embodiments may include some layered materials (e.g. a glucose oxidase layer, an analyte modulating layer etc.) while not including others (e.g. an interference rejection membrane, a protein layer, an adhesion promoting layer etc.). FIG. 2A illustrates a cross-section of one embodiment 100 of an element of the present invention, one that shows a plurality of layers coating a sensor electrode (e.g. the working electrode). This sensor embodiment is formed from a plurality of components that are typically in the form of layers of various conductive and non-conductive constituents disposed on each other according to art accepted methods and/or the specific methods of the invention disclosed herein. The components of the sensor are typically characterized herein as layers because, for example, it allows for a facile characterization of the sensor structure shown in FIG. 2A. Artisans will understand however, that in certain embodiments of the invention, the sensor constituents are combined such that multiple constituents form one or more heterogeneous layers. In this context, those of skill in the art understand that the ordering of the layered constituents can be altered in various embodiments of the invention.

The embodiment shown in FIG. 2A includes a base layer 102 to support the sensor 100. The base layer 102 can be made of a material such as a metal and/or a ceramic and/or a polymeric substrate, which may be self-supporting or further supported by another material as is known in the art. Embodiments of the invention include a conductive layer 104 which is disposed on and/or combined with the base layer 102. Typically the conductive layer 104 comprises one or more electrodes. An operating sensor 100 typically includes a plurality of electrodes such as a working electrode, a counter electrode and a reference electrode. Other embodiments may also include a plurality of working and/or counter and/or reference electrodes and/or one or more electrodes that performs multiple functions, for example one that functions as both as a reference and a counter electrode.

As discussed in detail below, the base layer 102 and/or conductive layer 104 can be generated using many known techniques and materials. In certain embodiments of the invention, the electrical circuit of the sensor is defined by etching the disposed conductive layer 104 into a desired pattern of conductive paths. A typical electrical circuit for the sensor 100 comprises two or more adjacent conductive paths with regions at a proximal end to form contact pads and regions at a distal end to form sensor electrodes. An electrically insulating cover layer 106 such as a polymer coating can be disposed on portions of the sensor 100. Acceptable polymer coatings for use as the insulating protective cover layer 106 can include, but are not limited to, non-toxic biocompatible polymers such as silicone compounds, polyimides, biocompatible solder masks, epoxy acrylate copolymers, or the like. In the sensors of the present invention, one or more exposed regions or apertures 108 can be made through the cover layer 106 to open the conductive layer 104 to the external environment and to, for example, allow an analyte such as glucose to permeate the layers of the sensor and be sensed by the sensing elements. Apertures 108 can be formed by a number of techniques, including laser ablation, tape masking, chemical milling or etching or photolithographic development or the like. In certain embodiments of the invention, during manufacture, a secondary photoresist can also be applied to the protective layer 106 to define the regions of the protective layer to be removed to form the aperture(s) 108. The exposed electrodes and/or contact pads can also undergo secondary processing (e.g. through the apertures 108), such as additional plating processing, to prepare the surfaces and/or strengthen the conductive regions.

In the sensor configuration shown in FIG. 2A, an analyte sensing layer 110 (which is typically a sensor chemistry layer, meaning that materials in this layer undergo a chemical reaction to produce a signal that can be sensed by the conductive layer) is disposed on one or more of the exposed electrodes of the conductive layer 104. In the sensor configuration shown in FIG. 2B, an interference rejection membrane 120 is disposed on one or more of the exposed electrodes of the conductive layer 104, with the analyte sensing layer 110 then being disposed on this interference rejection membrane 120. Typically, the analyte sensing layer 110 is an enzyme layer. Most typically, the analyte sensing layer 110 comprises an enzyme capable of producing and/or utilizing oxygen and/or hydrogen peroxide, for example the enzyme glucose oxidase. Optionally the enzyme in the analyte sensing layer is combined with a second carrier protein such as human serum albumin, bovine serum albumin or the like. In an illustrative embodiment, an oxidoreductase enzyme such as glucose oxidase in the analyte sensing layer 110 reacts with glucose to produce hydrogen peroxide, a compound which then modulates a current at an electrode. As this modulation of current depends on the concentration of hydrogen peroxide, and the concentration of hydrogen peroxide correlates to the concentration of glucose, the concentration of glucose can be determined by monitoring this modulation in the current. In a specific embodiment of the invention, the hydrogen peroxide is oxidized at a working electrode which is an anode (also termed herein the anodic working electrode), with the resulting current being proportional to the hydrogen peroxide concentration. Such modulations in the current caused by changing hydrogen peroxide concentrations can by monitored by any one of a variety of sensor detector apparatuses such as a universal sensor amperometric biosensor detector or one of the other variety of similar devices known in the art such as glucose monitoring devices produced by Medtronic MiniMed.

In embodiments of the invention, the analyte sensing layer 110 can be applied over portions of the conductive layer or over the entire region of the conductive layer. Typically the analyte sensing layer 110 is disposed on the working electrode which can be the anode or the cathode. Optionally, the analyte sensing layer 110 is also disposed on a counter and/or reference electrode. While the analyte sensing layer 110 can be up to about 1000 microns (μm) in thickness, typically the analyte sensing layer is relatively thin as compared to those found in sensors previously described in the art, and is for example, typically less than 1, 0.5, 0.25 or 0.1 microns in thickness. As discussed in detail below, some methods for generating a thin analyte sensing layer 110 include brushing the layer onto a substrate (e.g. the reactive surface of a platinum black electrode), as well as spin coating processes, dip and dry processes, low shear spraying processes, ink-jet printing processes, silk screen processes and the like.

Typically, the analyte sensing layer 110 is coated and or disposed next to one or more additional layers. Optionally, the one or more additional layers include a protein layer 116 disposed upon the analyte sensing layer 110. Typically, the protein layer 116 comprises a protein such as human serum albumin, bovine serum albumin or the like. Typically, the protein layer 116 comprises human serum albumin. In some embodiments of the invention, an additional layer includes an analyte modulating layer 112 that is disposed above the analyte sensing layer 110 to regulate analyte access with the analyte sensing layer 110. For example, the analyte modulating membrane layer 112 can comprise a glucose limiting membrane, which regulates the amount of glucose that contacts an enzyme such as glucose oxidase that is present in the analyte sensing layer. Such glucose limiting membranes can be made from a wide variety of materials known to be suitable for such purposes, e.g., silicone compounds such as polydimethyl siloxanes, polyurethanes, polyurea cellulose acetates, NAFION, polyester sulfonic acids (e.g. Kodak AQ), hydrogels or any other suitable hydrophilic membranes known to those skilled in the art. In certain embodiments of the invention, the glucose limiting membrane comprises a blended mixture of a linear polyurethane/polyurea polymer, and a branched acrylate polymer as disclosed for example in U.S. patent application Ser. No. 12/643,790, the contents of which are incorporated by reference.

In typical embodiments of the invention, an adhesion promoter layer 114 is disposed between the analyte modulating layer 112 and the analyte sensing layer 110 as shown in FIG. 2 in order to facilitate their contact and/or adhesion. In a specific embodiment of the invention, an adhesion promoter layer 114 is disposed between the analyte modulating layer 112 and the protein layer 116 as shown in FIG. 2 in order to facilitate their contact and/or adhesion. The adhesion promoter layer 114 can be made from any one of a wide variety of materials known in the art to facilitate the bonding between such layers. Typically, the adhesion promoter layer 114 comprises a silane compound. In alternative embodiments, protein or like molecules in the analyte sensing layer 110 can be sufficiently crosslinked or otherwise prepared to allow the analyte modulating membrane layer 112 to be disposed in direct contact with the analyte sensing layer 110 in the absence of an adhesion promoter layer 114.

Embodiments of typical elements used to make the sensors disclosed herein are discussed below.

### Typical Analyte Sensor Constituents Used in Embodiments of the Invention

The following disclosure provides examples of typical elements/constituents used in sensor embodiments of the invention. While these elements can be described as discreet units (e.g. layers), those of skill in the art understand that sensors can be designed to contain elements having a combination of some or all of the material properties and/or functions of the elements/constituents discussed below (e.g. an element that serves both as a supporting base constituent and/or a conductive constituent and/or a matrix for the analyte sensing constituent and which further functions as an electrode in the sensor). Those in the art understand that these thin film analyte sensors can be adapted for use in a number of sensor systems such as those described below.

### Base Constituent

Sensors of the invention typically include a base constituent (see, e.g. element 102 in FIG. 2A). The term “base constituent” is used herein according to art accepted terminology and refers to the constituent in the apparatus that typically provides a supporting matrix for the plurality of constituents that are stacked on top of one another and comprise the functioning sensor. In one form, the base constituent comprises a thin film sheet of insulative (e.g. electrically insulative and/or water impermeable) material. This base constituent can be made of a wide variety of materials having desirable qualities such as dielectric properties, water impermeability and hermeticity. Some materials include metallic, and/or ceramic and/or polymeric substrates or the like.

The base constituent may be self-supporting or further supported by another material as is known in the art. In one embodiment of the sensor configuration shown in FIG. 2A, the base constituent 102 comprises a ceramic. Alternatively, the base constituent comprises a polymeric material such as a polyimmide. In an illustrative embodiment, the ceramic base comprises a composition that is predominantly Al₂O₃ (e.g. 96%). The use of alumina as an insulating base constituent for use with implantable devices is disclosed in U.S. Pat. Nos. 4,940,858, 4,678,868 and 6,472,122 which are incorporated herein by reference. The base constituents of the invention can further include other elements known in the art, for example hermetical vias (see, e.g. WO 03/023388). Depending upon the specific sensor design, the base constituent can be relatively thick constituent (e.g. thicker than 50, 100, 200, 300, 400, 500 or 1000 microns). Alternatively, one can utilize a nonconductive ceramic, such as alumina, in thin constituents, e.g., less than about 30 microns.

### Conductive Constituent

The electrochemical sensors of the invention typically include a conductive constituent disposed upon the base constituent that includes at least one electrode for measuring an analyte or its byproduct (e.g. oxygen and/or hydrogen peroxide) to be assayed (see, e.g. element 104 in FIG. 2A). The term “conductive constituent” is used herein according to art accepted terminology and refers to electrically conductive sensor elements such as electrodes which are capable of measuring and a detectable signal and conducting this to a detection apparatus. An illustrative example of this is a conductive constituent that can measure an increase or decrease in current in response to exposure to a stimuli such as the change in the concentration of an analyte or its byproduct as compared to a reference electrode that does not experience the change in the concentration of the analyte, a coreactant (e.g. oxygen) used when the analyte interacts with a composition (e.g. the enzyme glucose oxidase) present in analyte sensing constituent 110 or a reaction product of this interaction (e.g. hydrogen peroxide). Illustrative examples of such elements include electrodes which are capable of producing variable detectable signals in the presence of variable concentrations of molecules such as hydrogen peroxide or oxygen. Typically one of these electrodes in the conductive constituent is a working electrode, which can be made from non-corroding metal. A metallic working electrode may be made from platinum group metals, including palladium or gold, or a non-corroding metallically conducting oxide, such as ruthenium dioxide. Alternatively the electrode may comprise a silver/silver chloride electrode composition. The working electrode may be constructed to be planar, a wire or a thin conducting film applied to a substrate, for example, by coating or printing. Typically, only a portion of the surface of the metallic conductor is in electrolytic contact with the analyte-containing solution. This portion is called the working surface of the electrode. The remaining surface of the electrode is typically isolated from the solution by an electrically insulating cover constituent 106. Examples of useful materials for generating this protective cover constituent 106 include polymers such as polyimides, polytetrafluoroethylene, polyhexafluoropropylene and silicones such as polysiloxanes.

In addition to the working electrode, the analyte sensors of the invention typically include a reference electrode or a combined reference and counter electrode (also termed a quasi-reference electrode or a counter/reference electrode). If the sensor does not have a counter/reference electrode then it may include a separate counter electrode, which may be made from the same or different materials as the working electrode. Typical sensors of the present invention have one or more working electrodes and one or more counter, reference, and/or counter/reference electrodes. One embodiment of the sensor of the present invention has two, three or four or more working electrodes. These working electrodes in the sensor may be integrally connected or they may be kept separate.

Typically for in vivo use, embodiments of the present invention are implanted subcutaneously in the skin of a mammal for direct contact with the body fluids of the mammal, such as blood. Alternatively the sensors can be implanted into other regions within the body of a mammal such as in the intraperotineal space. When multiple working electrodes are used, they may be implanted together or at different positions in the body. The counter, reference, and/or counter/reference electrodes may also be implanted either proximate to the working electrode(s) or at other positions within the body of the mammal.

### Interference Rejection Constituent

The electrochemical sensors of the invention optionally include an interference rejection constituent disposed between the surface of the electrode and the environment to be assayed. In particular, certain sensor embodiments rely on the oxidation and/or reduction of hydrogen peroxide generated by enzymatic reactions on the surface of a working electrode at a constant potential applied. Because amperometric detection based on direct oxidation of hydrogen peroxide requires a relatively high oxidation potential, sensors employing this detection scheme may suffer interference from oxidizable species that are present in biological fluids such as ascorbic acid, uric acid and acetaminophen. In this context, the term “interference rejection constituent” is used herein according to art accepted terminology and refers to a coating or membrane in the sensor that functions to inhibit spurious signals generated by such oxidizable species which interfere with the detection of the signal generated by the analyte to be sensed. Certain interference rejection constituents function via size exclusion (e.g. by excluding interfering species of a specific size). Examples of interference rejection constituents include one or more layers or coatings of compounds such as the hydrophilic crosslinked pHEMA and/or polylysine polymers disclosed in U.S. patent application Ser. No. 12/572,087, the contents of which are incorporated by reference, as well as cellulose acetate (including cellulose acetate incorporating agents such as poly(ethylene glycol)), polyethersulfones, polytetra-fluoroethylenes, the perfluoronated ionomer NAFION, polyphenylenediamine, epoxy and the like.

### Analyte Sensing Constituent

The electrochemical sensors of the invention include an analyte sensing constituent disposed on the electrodes of the sensor (see, e.g. element 110 in FIG. 2A). The term “analyte sensing constituent” is used herein according to art accepted terminology and refers to a constituent comprising a material that is capable of recognizing or reacting with an analyte whose presence is to be detected by the analyte sensor apparatus. Typically this material in the analyte sensing constituent produces a detectable signal after interacting with the analyte to be sensed, typically via the electrodes of the conductive constituent. In this regard the analyte sensing constituent and the electrodes of the conductive constituent work in combination to produce the electrical signal that is read by an apparatus associated with the analyte sensor. Typically, the analyte sensing constituent comprises an oxidoreductase enzyme capable of reacting with and/or producing a molecule whose change in concentration can be measured by measuring the change in the current at an electrode of the conductive constituent (e.g. oxygen and/or hydrogen peroxide), for example the enzyme glucose oxidase. An enzyme capable of producing a molecule such as hydrogen peroxide can be disposed on the electrodes according to a number of processes known in the art. The analyte sensing constituent can coat all or a portion of the various electrodes of the sensor. In this context, the analyte sensing constituent may coat the electrodes to an equivalent degree. Alternatively the analyte sensing constituent may coat different electrodes to different degrees, with for example the coated surface of the working electrode being larger than the coated surface of the counter and/or reference electrode.

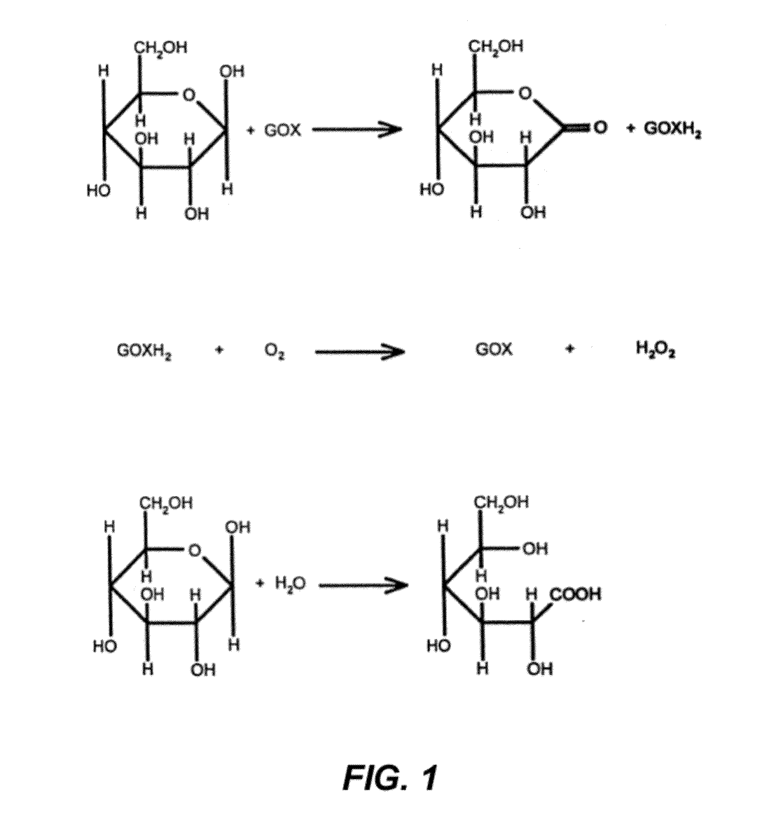

Typical sensor embodiments of this element of the invention utilize an enzyme (e.g. glucose oxidase) that has been combined with a second protein (e.g. albumin) in a fixed ratio (e.g. one that is typically optimized for glucose oxidase stabilizing properties) and then applied on the surface of an electrode to form a thin enzyme constituent. In a typical embodiment, the analyte sensing constituent comprises a GOx and HSA mixture. In a typical embodiment of an analyte sensing constituent having GOx, the GOx reacts with glucose present in the sensing environment (e.g. the body of a mammal) and generates hydrogen peroxide according to the reaction shown in FIG. 1, wherein the hydrogen peroxide so generated is anodically detected at the working electrode in the conductive constituent. Illustrative GOx compositions having different amounts of this enzyme are disclosed for example in U.S. patent application Ser. No. 13/010,640, the contents of which are incorporated by reference herein.

As noted above, the enzyme and the second protein (e.g. an albumin) are typically treated to form a crosslinked matrix (e.g. by adding a cross-linking agent to the protein mixture). As is known in the art, crosslinking conditions may be manipulated to modulate factors such as the retained biological activity of the enzyme, its mechanical and/or operational stability. Illustrative crosslinking procedures are described in U.S. patent application Ser. No. 10/335,506 and PCT publication WO 03/035891 which are incorporated herein by reference. For example, an amine cross-linking reagent, such as, but not limited to, glutaraldehyde, can be added to the protein mixture.

### Protein Constituent

The electrochemical sensors of the invention optionally include a protein constituent disposed between the analyte sensing constituent and the analyte modulating constituent (see, e.g. element 116 in FIG. 2A). The term “protein constituent” is used herein according to art accepted terminology and refers to constituent containing a carrier protein or the like that is selected for compatibility with the analyte sensing constituent and/or the analyte modulating constituent. In typical embodiments, the protein constituent comprises an albumin such as human serum albumin. The HSA concentration may vary between about 0.5%-30% (w/v). Typically the HSA concentration is about 1-10% w/v, and most typically is about 5% w/v. In alternative embodiments of the invention, collagen or BSA or other structural proteins used in these contexts can be used instead of or in addition to HSA. This constituent is typically crosslinked on the analyte sensing constituent according to art accepted protocols.

### Adhesion Promoting Constituent

The electrochemical sensors of the invention can include one or more adhesion promoting (AP) constituents (see, e.g. element 114 in FIG. 2A). The term “adhesion promoting constituent” is used herein according to art accepted terminology and refers to a constituent that includes materials selected for their ability to promote adhesion between adjoining constituents in the sensor. Typically, the adhesion promoting constituent is disposed between the analyte sensing constituent and the analyte modulating constituent. Typically, the adhesion promoting constituent is disposed between the optional protein constituent and the analyte modulating constituent. The adhesion promoter constituent can be made from any one of a wide variety of materials known in the art to facilitate the bonding between such constituents and can be applied by any one of a wide variety of methods known in the art. Typically, the adhesion promoter constituent comprises a silane compound such as γ-aminopropyltrimethoxysilane.

The use of silane coupling reagents, especially those of the formula R′Si(OR)₃ in which R′ is typically an aliphatic group with a terminal amine and R is a lower alkyl group, to promote adhesion is known in the art (see, e.g. U.S. Pat. No. 5,212,050 which is incorporated herein by reference). For example, chemically modified electrodes in which a silane such as γ-aminopropyltriethoxysilane and glutaraldehyde were used in a step-wise process to attach and to co-crosslink bovine serum albumin (BSA) and glucose oxidase (GOx) to the electrode surface are well known in the art (see, e.g. Yao, T. Analytica Chim. Acta 1983, 148, 27-33).

In certain embodiments of the invention, the adhesion promoting constituent further comprises one or more compounds that can also be present in an adjacent constituent such as the polydimethyl siloxane (PDMS) compounds that serves to limit the diffusion of analytes such as glucose through the analyte modulating constituent. In illustrative embodiments the formulation comprises 0.5-20% PDMS, typically 5-15% PDMS, and most typically 10% PDMS. In certain embodiments of the invention, the adhesion promoting constituent is crosslinked within the layered sensor system and correspondingly includes an agent selected for its ability to crosslink a moiety present in a proximal constituent such as the analyte modulating constituent. In illustrative embodiments of the invention, the adhesion promoting constituent includes an agent selected for its ability to crosslink an amine or carboxyl moiety of a protein present in a proximal constituent such a the analyte sensing constituent and/or the protein constituent and or a siloxane moiety present in a compound disposed in a proximal layer such as the analyte modulating layer.

### Analyte Modulating Constituent

The electrochemical sensors of the invention include an analyte modulating constituent disposed on the sensor (see, e.g. element 112 in FIG. 2A). The term “analyte modulating constituent” is used herein according to art accepted terminology and refers to a constituent that typically forms a membrane on the sensor that operates to modulate the diffusion of one or more analytes, such as glucose, through the membrane. In certain embodiments of the invention, the analyte modulating constituent is an analyte-limiting membrane (e.g. a glucose limiting membrane) which operates to prevent or restrict the diffusion of one or more analytes, such as glucose, through the constituents. In other embodiments of the invention, the analyte-modulating constituent operates to facilitate the diffusion of one or more analytes, through the constituents. Optionally such analyte modulating constituents can be formed to prevent or restrict the diffusion of one type of molecule through the constituent (e.g. glucose), while at the same time allowing or even facilitating the diffusion of other types of molecules through the constituent (e.g. O₂).

With respect to glucose sensors, in known enzyme electrodes, glucose and oxygen from blood, as well as some interferents, such as ascorbic acid and uric acid, diffuse through a primary membrane of the sensor. As the glucose, oxygen and interferents reach the analyte sensing constituent, an enzyme, such as glucose oxidase, catalyzes the conversion of glucose to hydrogen peroxide and gluconolactone. The hydrogen peroxide may diffuse back through the analyte modulating constituent, or it may diffuse to an electrode where it can be reacted to form oxygen and a proton to produce a current that is proportional to the glucose concentration. The sensor membrane assembly serves several functions, including selectively allowing the passage of glucose therethrough. In this context, an illustrative analyte modulating constituent is a semi-permeable membrane which permits passage of water, oxygen and at least one selective analyte and which has the ability to absorb water, the membrane having a water soluble, hydrophilic polymer.

A variety of illustrative analyte modulating compositions are known in the art and are described for example in U.S. patent application Ser. No. 12/643,790, U.S. Pat. Nos. 6,319,540, 5,882,494, 5,786,439 5,777,060, 5,771,868 and 5,391,250, and U.S. patent application Ser. No. 12/643,790, the disclosures of each being incorporated herein by reference. In certain embodiments of the invention, the analyte modulating layer comprises a blended mixture of a linear polyurethane/polyurea polymer, and a branched acrylate polymer that are blended together at a ratio of between 1:1 and 1:20 by weight %. In one illustrative embodiment, the analyte modulating layer comprises a polyurethane/polyurea polymer formed from a mixture comprising a diisocyanate; a hydrophilic polymer comprising a hydrophilic diol or hydrophilic diamine; and a siloxane having an amino, hydroxyl or carboxylic acid functional group at a terminus that is blended together in a 1:1 to 1:2 ratio with a branched acrylate polymer formed from a mixture comprising a butyl, propyl, ethyl or methyl-acrylate; an amino-acrylate; and a siloxane-acrylate; and a poly(ethylene oxide)-acrylate.

### Cover Constituent

The electrochemical sensors of the invention include one or more cover constituents which are typically electrically insulating protective constituents (see, e.g. element 106 in FIG. 2A). Typically, such cover constituents can be in the form of a coating, sheath or tube and are disposed on at least a portion of the analyte modulating constituent. Acceptable polymer coatings for use as the insulating protective cover constituent can include, but are not limited to, non-toxic biocompatible polymers such as silicone compounds, polyimides, biocompatible solder masks, epoxy acrylate copolymers, or the like. Further, these coatings can be photo-imageable to facilitate photolithographic forming of apertures through to the conductive constituent. A typical cover constituent comprises spun on silicone. As is known in the art, this constituent can be a commercially available RTV (room temperature vulcanized) silicone composition. A typical chemistry in this context is polydimethyl siloxane (acetoxy based).

Typical Elements Useful with Embodiments of the Invention

Embodiments of the sensor elements and sensors disclosed herein can be operatively coupled to a variety of other systems elements typically used with analyte sensors (e.g. structural elements such as piercing members, insertion sets and the like as well as electronic components such as processors, monitors, medication infusion pumps and the like), for example to adapt them for use in various contexts (e.g. implantation within a mammal). One embodiment of the invention includes a method of monitoring a physiological characteristic of a user using an embodiment of the invention that includes a plurality of input elements capable of receiving signals from multiple working electrodes having different material properties (e.g. signals based on a sensed physiological characteristic value of the user), and a processor for analyzing the received signals. In typical embodiments of the invention, the processor determines a dynamic behavior of the physiological characteristic value and provides an observable indicator based upon the dynamic behavior of the physiological characteristic value so determined. In some embodiments, the physiological characteristic value is a measure of the concentration of blood glucose in the user. In other embodiments, the process of analyzing the received signal and determining a dynamic behavior includes repeatedly measuring the physiological characteristic value to obtain a series of physiological characteristic values in order to, for example, incorporate comparative redundancies into a sensor apparatus in a manner designed to provide confirmatory information on sensor function, analyte concentration measurements, the presence of interferences and the like.

Embodiments of the invention include devices which display data from measurements of a sensed physiological characteristic (e.g. blood glucose concentrations) in a manner and format tailored to allow a user of the device to easily monitor and, if necessary, modulate the physiological status of that characteristic (e.g. modulation of blood glucose concentrations via insulin administration). An illustrative embodiment of the invention is a device comprising a sensor input capable of receiving a signal from a sensor, the signal being based on a sensed physiological characteristic value of a user; a memory for storing a plurality of measurements of the sensed physiological characteristic value of the user from the received signal from the sensor; and a display for presenting a text and/or graphical representation of the plurality of measurements of the sensed physiological characteristic value (e.g. text, a line graph or the like, a bar graph or the like, a grid pattern or the like or a combination thereof). Typically, the graphical representation displays real time measurements of the sensed physiological characteristic value. Such devices can be used in a variety of contexts, for example in combination with other medical apparatuses.

An illustrative system embodiment consists of a glucose sensor, a transmitter and pump receiver and a glucose meter. In this system, radio signals from the transmitter can be sent to the pump receiver periodically (e.g. every 5 minutes) to provide providing real-time sensor glucose (SG) values. Values/graphs are displayed on a monitor of the pump receiver so that a user can self monitor blood glucose and deliver insulin using their own insulin pump. Typically an embodiment of device disclosed herein communicates with a second medical device via a wired or wireless connection. Wireless communication can include for example the reception of emitted radiation signals as occurs with the transmission of signals via RF telemetry, infrared transmissions, optical transmission, sonic and ultrasonic transmissions and the like. Optionally, the device is an integral part of a medication infusion pump (e.g. an insulin pump). Typically in such devices, the physiological characteristic values includes a plurality of measurements of blood glucose.

### Illustrative Methods and Materials for Making Analyte Sensor Apparatus of the Invention

A number of articles, U.S. patents and patent application describe the state of the art with the common methods and materials disclosed herein and further describe various elements (and methods for their manufacture) that can be used in the sensor designs disclosed herein. These include for example, U.S. Pat. Nos. 6,413,393; 6,368,274; 5,786,439; 5,777,060; 5,391,250; 5,390,671; 5,165,407, 4,890,620, 5,390,671, 5,390,691, 5,391,250, 5,482,473, 5,299,571, 5,568,806; United States Patent Application 20020090738; as well as PCT International Publication Numbers WO 01/58348, WO 03/034902, WO 03/035117, WO 03/035891, WO 03/023388, WO 03/022128, WO 03/022352, WO 03/023708, WO 03/036255, WO03/036310 and WO 03/074107, the contents of each of which are incorporated herein by reference.

Typical sensors for monitoring glucose concentration of diabetics are further described in Shichiri, et al.: “In Vivo Characteristics of Needle-Type Glucose Sensor-Measurements of Subcutaneous Glucose Concentrations in Human Volunteers,” Horm. Metab. Res., Suppl. Ser. 20:17-20 (1988); Bruckel, et al.: “In Vivo Measurement of Subcutaneous Glucose Concentrations with an Enzymatic Glucose Sensor and a Wick Method,” Klin. Wochenschr. 67:491-495 (1989); and Pickup, et al.: “In Vivo Molecular Sensing in Diabetes Mellitus: An Implantable Glucose Sensor with Direct Electron Transfer,” Diabetologia 32:213-217 (1989). Other sensors are described in, for example Reach, et al., in ADVANCES IN IMPLANTABLE DEVICES, A. Turner (ed.), JAI Press, London, Chap. 1, (1993), incorporated herein by reference.

### General Methods for Making Analyte Sensors

A typical embodiment of the invention disclosed herein is a method of making a sensor electrode array for implantation within a mammal, for example one comprising the steps of: providing a base layer; forming a conductive layer on the base layer, wherein the conductive layer includes a plurality of electrodes (and typically a plurality of working electrodes having different material properties). In certain embodiments of the invention, the iridium comprising a working electrode is formed from a process designed to manipulate the surface of this electrode (e.g. the roughness of this surface), for example via an etching or sputtering process (see, e.g. U.S. Pat. Nos. 6,143,191, 6,018,065, U.S. Patent Application Nos. 20020075631 and 20060259109, and Wang et al., IEEE Trans Biomed Eng. 2009 January; 56(1): 6-14, the contents of which are incorporated herein by reference). Similarly, in certain embodiments of the invention, the iridium comprising a working electrode is formed from a process designed to manipulate the oxidation state of the iridium metal.

Embodiments for making the sensor systems disclosed herein further include forming a interference rejection membrane on the conductive layer, forming an analyte sensing layer on the interference rejection membrane, wherein the analyte sensing layer includes a composition that can alter the electrical current at the electrode in the conductive layer in the presence of an analyte; optionally forming a protein layer on the analyte sensing layer; forming an adhesion promoting layer on the analyte sensing layer or the optional protein layer; forming an analyte modulating layer disposed on the adhesion promoting layer, wherein the analyte modulating layer includes a composition that modulates the diffusion of the analyte therethrough; and forming a cover layer disposed on at least a portion of the analyte modulating layer, wherein the cover layer further includes an aperture over at least a portion of the analyte modulating layer. In embodiments of the invention, four sensor arrays can be disposed on two probes which are releasably coupled to a probes platform. In certain embodiments of the invention, the analyte modulating layer comprises a hydrophilic comb-copolymer having a central chain and a plurality of side chains coupled to the central chain, wherein at least one side chain comprises a silicone moiety. In some embodiments of these methods, the analyte sensor apparatus is formed in a planar geometric configuration

As disclosed herein, the various layers of the sensor can be manufactured to exhibit a variety of different characteristics which can be manipulated according to the specific design of the sensor. For example, the adhesion promoting layer includes a compound selected for its ability to stabilize the overall sensor structure, typically a silane composition. In some embodiments of the invention, the analyte sensing layer is formed by a spin coating process and is of a thickness selected from the group consisting of less than 1, 0.5, 0.25 and 0.1 microns in height.

Typically, a method of making the sensor includes the step of forming a protein layer on the analyte sensing layer, wherein a protein within the protein layer is an albumin selected from the group consisting of bovine serum albumin and human serum albumin. Typically, a method of making the sensor includes the step of forming an analyte sensing layer that comprises an enzyme composition selected from the group consisting of glucose oxidase, glucose dehydrogenase, lactate oxidase, hexokinase and lactate dehydrogenase. In such methods, the analyte sensing layer typically comprises a carrier protein composition in a substantially fixed ratio with the enzyme, and the enzyme and the carrier protein are distributed in a substantially uniform manner throughout the analyte sensing layer.

### Typical Protocols and Materials Useful in the Manufacture of Analyte Sensors

The disclosure provided herein includes sensors and sensor designs that can be generated using combinations of various well known techniques. The disclosure further provides methods for applying very thin enzyme coatings to these types of sensors as well as sensors produced by such processes. In this context, some embodiments of the invention include methods for making such sensors on a substrate according to art accepted processes. In certain embodiments, the substrate comprises a rigid and flat structure suitable for use in photolithographic mask and etch processes. In this regard, the substrate typically defines an upper surface having a high degree of uniform flatness. A polished glass plate may be used to define the smooth upper surface. Alternative substrate materials include, for example, stainless steel, aluminum, and plastic materials such as delrin, etc. In other embodiments, the substrate is non-rigid and can be another layer of film or insulation that is used as a substrate, for example plastics such as polyimides and the like.

An initial step in the methods of the invention typically includes the formation of a base layer of the sensor. The base layer can be disposed on the substrate by any desired means, for example by controlled spin coating. In addition, an adhesive may be used if there is not sufficient adhesion between the substrate layer and the base layer. A base layer of insulative material is formed on the substrate, typically by applying the base layer material onto the substrate in liquid form and thereafter spinning the substrate to yield the base layer of thin, substantially uniform thickness. These steps are repeated to build up the base layer of sufficient thickness, followed by a sequence of photolithographic and/or chemical mask and etch steps to form the conductors discussed below. In an illustrative form, the base layer comprises a thin film sheet of insulative material, such as ceramic or polyimide substrate. The base layer can comprise an alumina substrate, a polyimide substrate, a glass sheet, controlled pore glass, or a planarized plastic liquid crystal polymer. The base layer may be derived from any material containing one or more of a variety of elements including, but not limited to, carbon, nitrogen, oxygen, silicon, sapphire, diamond, aluminum, copper, gallium, arsenic, lanthanum, neodymium, strontium, titanium, yttrium, or combinations thereof. Additionally, the substrate may be coated onto a solid support by a variety of methods well-known in the art including physical vapor deposition, or spin-coating with materials such as spin glasses, chalcogenides, graphite, silicon dioxide, organic synthetic polymers, and the like.

The methods of the invention further include the generation of a conductive layer having one or more sensing elements. Typically these sensing elements are electrodes that are formed by one of the variety of methods known in the art such as photoresist, etching and rinsing to define the geometry of the active electrodes. The electrodes can then be made electrochemically active, for example by the sputtering of an iridium composition for the working electrode and/or the electrodeposition of Pt black for the working and counter electrode, and silver followed by silver chloride on the reference electrode. A sensor layer such as a analyte sensing enzyme layer can then be disposed on the sensing layer by electrochemical deposition or a method other than electrochemical deposition such a spin coating, followed by vapor crosslinking, for example with a dialdehyde (glutaraldehyde) or a carbodi-imide.

Electrodes of the invention can be formed from a wide variety of materials known in the art. For example, the electrode may be made of a noble late transition metals. Metals such as gold, platinum, silver, rhodium, iridium, ruthenium, palladium, or osmium can be suitable in various embodiments of the invention. Of these metals, silver, gold, or platinum is typically used as a reference electrode metal. A silver electrode which is subsequently chloridized is typically used as the reference electrode. These metals can be deposited by any means known in the art, including the plasma deposition method cited, supra, or by an electroless method which may involve the deposition of a metal onto a previously metallized region when the substrate is dipped into a solution containing a metal salt and a reducing agent. The electroless method proceeds as the reducing agent donates electrons to the conductive (metallized) surface with the concomitant reduction of the metal salt at the conductive surface. The result is a layer of adsorbed metal. (For additional discussions on electroless methods, see: Wise, E. M. Palladium: Recovery, Properties, and Uses, Academic Press, New York, N.Y. (1988); Wong, K. et al. Plating and Surface Finishing 1988, 75, 70-76; Matsuoka, M. et al. Ibid. 1988, 75, 102-106; and Pearlstein, F. “Electroless Plating,” Modern Electroplating, Lowenheim, F. A., Ed., Wiley, New York, N.Y. (1974), Chapter 31). Such a metal deposition process must yield a structure with good metal to metal adhesion and minimal surface contamination, however, to provide a catalytic metal electrode surface with a high density of active sites. Such a high density of active sites is a property necessary for the efficient redox conversion of an electroactive species such as hydrogen peroxide.

In an exemplary embodiment of the invention, the base layer is initially coated with a thin film conductive layer by electrode deposition, surface sputtering, or other suitable process step. In one embodiment this conductive layer may be provided as a plurality of thin film conductive layers, such as an initial chrome-based layer suitable for chemical adhesion to a polyimide base layer followed by subsequent formation of thin film gold-based and chrome-based layers in sequence. In alternative embodiments, other electrode layer conformations or materials can be used. The conductive layer is then covered, in accordance with conventional photolithographic techniques, with a selected photoresist coating, and a contact mask can be applied over the photoresist coating for suitable photoimaging. The contact mask typically includes one or more conductor trace patterns for appropriate exposure of the photoresist coating, followed by an etch step resulting in a plurality of conductive sensor traces remaining on the base layer. In an illustrative sensor construction designed for use as a subcutaneous glucose sensor, each sensor trace can include three parallel sensor elements corresponding with three separate electrodes such as a working electrode, a counter electrode and a reference electrode.

Portions of the conductive sensor layers are typically covered by an insulative cover layer, typically of a material such as a silicon polymer and/or a polyimide. The insulative cover layer can be applied in any desired manner. In an exemplary procedure, the insulative cover layer is applied in a liquid layer over the sensor traces, after which the substrate is spun to distribute the liquid material as a thin film overlying the sensor traces and extending beyond the marginal edges of the sensor traces in sealed contact with the base layer. This liquid material can then be subjected to one or more suitable radiation and/or chemical and/or heat curing steps as are known in the art. In alternative embodiments, the liquid material can be applied using spray techniques or any other desired means of application. Various insulative layer materials may be used such as photoimagable epoxyacrylate, with an illustrative material comprising a photoimagable polyimide available from OCG, Inc. of West Paterson, N.J., under the product number 7020.

### Kits and Sensor Sets of the Invention

In another embodiment of the invention, a kit and/or sensor set, useful for the sensing an analyte as is described above, is provided. The kit and/or sensor set typically comprises a container, a label and an analyte sensor as described above. Suitable containers include, for example, an easy to open package made from a material such as a metal foil, bottles, vials, syringes, and test tubes. The containers may be formed from a variety of materials such as metals (e.g. foils) paper products, glass or plastic. The label on, or associated with, the container indicates that the sensor is used for assaying the analyte of choice. In some embodiments, the container holds an amperometric glucose sensor comprising a first working electrode consisting essentially of an iridium composition and a second working electrode consisting essentially of a platinum composition. The kit and/or sensor set may further include other materials desirable from a commercial and user standpoint, including elements or devices designed to facilitate the introduction of the sensor into the analyte environment, other buffers, diluents, filters, needles, syringes, and package inserts with instructions for use.

1. An amperometric glucose sensor system comprising:
a processor;
a first working electrode comprising a first electrochemically reactive surface formed from an iridium composition, wherein the first electrochemically reactive surface generates an electrochemical signal that is assessed by the processor in the presence of glucose;
a second working electrode comprising a second electrochemically reactive surface formed from a platinum composition, wherein the second electrochemically reactive surface generates an electrochemical signal that is assessed by the processor in the presence of glucose;
a counter electrode;
a reference electrode; and
a computer-readable program code having instructions, which when executed cause the processor to:
assess electrochemical signal data obtained from the first working electrode and the second working electrode; and
compute a glucose concentration based upon the electrochemical signal data obtained from the first working electrode and/or the second working electrode.

2. The amperometric glucose sensor system of claim 1, wherein the first working electrode and the second working electrode are coated with:
a glucose oxidase layer; and/or
an interference rejection layer; and/or
a glucose modulating layer, wherein the glucose modulating layer comprises a composition that modulates the diffusion of glucose through the glucose modulating layer.

3. The amperometric glucose sensor system of claim 2, wherein:
the first working electrode is not coated with an interference rejection layer and the second working electrode is coated with a interference rejection layer; and/or
the first working electrode is coated with a first glucose oxidase layer and the second working electrode is coated with a second glucose oxidase layer, wherein the amount of glucose oxidase in the first glucose oxidase layer is greater than the amount of glucose oxidase in the second glucose oxidase layer; and/or
the first working electrode is coated with a first glucose modulating layer having a first rate of glucose diffusion and the second working electrode is coated with a second glucose modulating layer having a second rate of glucose diffusion, wherein the first rate of glucose diffusion is less than the second rate of glucose diffusion.

4. The amperometric glucose sensor system of claim 1, wherein:
the first electrochemically reactive surface comprises iridium oxide; and/or
the second electrochemically reactive surface comprises platinum black.

5. The amperometric glucose sensor system of claim 1, wherein the first working electrode exhibits an electrochemically reactive surface area that is at least 25% greater than a geometrical surface area of the electrochemically reactive surface.

6. The amperometric glucose sensor system of claim 1, wherein the first and/or second working electrodes are formed from a cylindrical wire having a diameter less than 0.0015 inches.

7. The amperometric glucose sensor system of claim 1, wherein the processor evaluates data resulting from a plurality of different voltages applied to the system.

8. The amperometric glucose sensor system of claim 7, wherein:
the processor evaluates data resulting from a plurality different voltage pulses applied to the first working electrode and second working electrode; and the data evaluated results from:
a voltage potential of between 0.2 and 0.6 volts applied to the first working electrode; and
a voltage potential of between 0.5 and 0.7 volts applied to the second working electrode.

9. The amperometric glucose sensor system of claim 1, wherein the processor compares the electrochemical signal data from the first working electrode and the second working electrode and the comparison includes:
observing whether a signal obtained from the first working electrode and the second working electrode falls within a predetermined range of values;
observing a trend in sensor signal data from the first working electrode and the second working electrode; or
observing an amount of nonspecific signal noise in the first working electrode and the second working electrode.

10. The amperometric glucose sensor system of claim 1, wherein the processor:
assesses electrochemical signal data obtained from the first working electrode and the second working against one or more reliability parameters;
ranks electrochemical signal data obtained from the first working electrode and the second working electrode; and
computes glucose concentration based upon the ranking of electrochemical signal data obtained from the first working electrode and the second working electrode.

11. The amperometric glucose sensor system of claim 1, wherein the system further comprises:
a first probe adapted to be inserted in vivo, wherein the first probe includes a first electrode array comprising the first working electrode and the second working electrode:
a probe platform coupled to the first probe;
a second probe coupled to the probe platform and adapted to be inserted in vivo, wherein the second probe comprises a second electrode array comprising:
a third working electrode comprising a third electrochemically reactive surface formed from an iridium composition, wherein the third electrochemically reactive surface generates an electrochemical signal that is observed by the processor in the presence of glucose;
a fourth working electrode comprising a fourth electrochemically reactive surface formed from a platinum composition, wherein the fourth electrochemically reactive surface generates an electrochemical signal that is observed by the processor in the presence of glucose; and
a computer-readable program code having instructions, which when executed cause the processor to:
assess electrochemical signal data obtained from the first and second electrode arrays; and
compute a glucose concentration based upon the electrochemical signal data obtained from the first and second electrode arrays.

12. The amperometric glucose sensor system of claim 11, wherein:
electrochemical signal data obtained from the first working electrode and the second working electrode is weighted according to one or more reliability parameters and the weighted electrochemical signal data is fused to compute a glucose concentration; and/or
electrochemical signal data obtained from the first and second electrode arrays is weighted according to one or more reliability parameters and the weighted electrochemical signal data is fused to compute a glucose concentration.

13. The amperometric glucose sensor system of claim 11, wherein:
the first and second probes are oriented on the probe platform so that the first and second electrode arrays are located at different depths when inserted into an in vivo environment; and/or
the first probe and second probes are coupled to the probe platform and the probe platform is made from a flexible material that allows the probes to twist and bend when implanted in vivo in a manner that inhibits in vivo movement of the probes.

14. The amperometric glucose sensor system of claim 11, wherein the system further comprises an adhesive patch adapted to secure the probe platform to skin of a diabetic patient

15. A method for computing a blood glucose concentration in a diabetic patient, the method comprising:
observing electrochemical signal data generated by a sensor system comprising:
a processor;
a first working electrode comprising a first electrochemically reactive surface formed from an iridium composition, wherein the first electrochemically reactive surface generates an electrochemical signal that is assessed by the processor in the presence of glucose;
a second working electrode comprising a second electrochemically reactive surface formed from a platinum composition, wherein the second electrochemically reactive surface generates an electrochemical signal that is assessed by the processor in the presence of glucose;
a counter electrode;
a reference electrode; and
a computer-readable program code having instructions, which when executed cause the processor to:
assess electrochemical signal data obtained from the first working electrode and the second working electrode; and
compute a glucose concentration based upon the electrochemical signal data obtained from the first working electrode and the second working electrode;
wherein the first working electrode and second working electrode are configured to be electronically independent of one another; and the method further comprises:
comparing the electrochemical signal data from the first working electrode and the second working electrode; and
computing a blood glucose concentration using the comparison of the electrochemical signal data obtained from the first working electrode and the second working electrode.

16. The method of claim 15, wherein the comparison of electrochemical signal data from the first working electrode and the second working electrode includes:
observing whether a signal obtained from the first working electrode and the second working electrode falls within a predetermined range of values;
observing a trend in sensor signal data from the first working electrode and the second working electrode; or
observing an amount of nonspecific signal noise in the first working electrode and the second working electrode.

17. The method of claim 15, wherein the comparison of electrochemical signal data from the first working electrode and the second working electrode is used to identify one or more signals that is:
indicative of increasing glucose blood concentrations or decreasing blood glucose concentrations in the diabetic patient;
indicative of a presence of interfering compounds;
indicative of background noise;
indicative of sensor hydration;
indicative of sensor signal drift; and/or
indicative of sensor loss of sensitivity to glucose.

18. The method of claim 15, further comprising using a monitor adapted to display discreet signal information from the first working electrode and/or the second working electrode.

19. A composition of matter comprising:
an iridium composition having an electrochemically reactive surface;
a glucose oxidase composition disposed upon the electrochemically reactive surface;
an analyte modulating layer disposed upon the glucose oxidase composition, wherein the analyte modulating layer comprises:
a linear polyurethane/polyurea polymer;
a branched acrylate polymer; or
a blended mixture of the linear polyurethane/polyurea polymer and the branched acrylate polymer, wherein the mixture is blended at a ratio of between 1:1 and 1:20 by weight percentage.

20. The composition of claim 19, wherein:
(1) the linear polyurethane/polyurea polymer is formed from a mixture comprising:
(a) a diisocyanate;
(b) at least one hydrophilic diol or hydrophilic diamine; and
(c) a siloxane; and/or
(2) the branched acrylate polymer is formed from a mixture comprising:
(a) a 2-(dimethylamino)ethyl methacrylate;
(b) a methyl methacrylate;
(c) a polydimethyl siloxane monomethacryloxypropyl; and
(d) a poly(ethylene oxide) methyl ether methacrylate.
